SEQUENCE LISTING (107 sequences)

SEQ ID NO: 1 - protein, 95 aa, Plasmodium falciparum
ISQHQCVKKQCPENSGCFRHLDEREECKCLLNYKQEGDKCVENPNPTCNENNGGCDADATCTEEDSGSSRKKITCECTKPDSYPLFDGIFCSSSN

SEQ ID NO: 2 - protein, 95 aa, Plasmodium falciparum
ISQHQCVKKQCPENSGCFRHLDEREECKCLLNYKQEGDKCVENPNPTCNENNGGCDADAKCTEEDSGSNGKKITCECTKPDSYPLFDGIFCSSSN

SEQ ID NO: 3 - protein, 94 aa, Plasmodium falciparum
ISQHQCVKKQCPQNSGCFRHLDEREECKCLLNYKQEGDKCVENPNPTCNENNGGCDADAKCTEEDSGSNGKKITCECTKPDSYPLFDGIFCSSS

SEQ ID NO: 4 - protein, 95 aa, Plasmodium falciparum
ISQHQCVKKQCPQNSGCFRHLDEREECKCLLNYKQEGDKCVENPNPTCNENNGGCDADAKCTEEDSGSNGKKITCECTKPDSYPFFDGIFCSSSN

SEQ ID NO: 5 - protein, 398 aa, Plasmodium falciparum
ISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSN

SEQ ID NO: 6 - protein, 169 aa, Plasmodium falciparum
VTVDTVCKRGFLIQMSGHLECKCENDLVLVNEETCEEKVLKCDEKTVNKPCGDFSKCIKIDGNPVSYACKCNLGYDMVNNVCIPNECKNVACGNGKCILDTSNPVKTGVCSCNIGKVPNVQDQKCSKDGETKCSLKCLKENEACKAVDGIYKCDCKDGFIIDNEASICT

SEQ ID NO: 7 - protein, 169 aa, Plasmodium falciparum
VTVDTVCKRGFLIQMSGHLECKCENDTVLVNEETCEEKVLKCDEKTVNKPCGDFSKCIKIDGNPVSYACKCNLGYDMVNNVCIPNECKNVTCGNGKCILDTSNPVKTGVCSCNIGKVPNVQDQKCSKDGETKCSLKCLKENETCKAVDGIYKCDCKDGFIIDNEASICT

SEQ ID NO: 8 - protein, 168 aa, Plasmodium falciparum
VTENTICKYGYLIQMSNHYECKCIEGYVLINEDTCGKKVVCDKVENSFKACDEYAYCFDLGNKNNEKQIKCMCRTEYTLTAGVCVPNVCRDKVCGKGKCIVDPANSLTHTCSCNIGTILNQNKLCDIQGDTPCSLKCAENEVCTLEGNYYTCKEDPSSNGGGNTVDQA

SEQ ID NO: 9 - protein, 74 aa, Plasmodium falciparum
PSDKHIEQYLKKIQNSLSTEWSPCSVTCGNGIQVRIKPGSANKPKDELDYENDIEKKICKMEKCSSVFNVVNSS

SEQ ID NO: 10 - protein, 51 aa, Plasmodium falciparum
EKTASCGVWDEWSPCSVTCGKGTRSRKREILHEGCTSELQEQCEEERCLPK

SEQ ID NO: 11 - protein, 74 aa, Plasmodium falciparum
THDTCDEWSEWSACTHGISTRKCLSDSSIKDETLVCTKCDKWGEWSECKDGRMHRKVLNCPFIKEEQECDVNNE

SEQ ID NO: 12 - protein, 65 aa, Plasmodium falciparum
FYSEWGEWSNCAMDCDHPDNVQIRERECIHPSGDCFKGDLKESRPCIIPLPCNELFSHKDNSAFK

SEQ ID NO: 13 - protein, 283 aa, Plasmodium falciparum
PSDKHIEQYLKKIQNSLSTEWSPCSVTCGNGIQVRIKPGSANKPKDELDYENDIEKKICKMEKCSSVFNVVNSSAAVAMAEKTASCGVWDEWSPCSVTCGKGTRSRKREILHEGCTSELQEQCEEERCLPKAAVAMATHDTCDEWSEWSACTHGISTRKCLSDSSIKDETLVCTKCDKWGEWSECKDGRMHRKVLNCPFIKEEQECDVNNEAAVAMAFYSEWGEWSNCAMDCDHPDNVQIRERECIHPSGDCFKGDLKESRPCIIPLPPCNELFSHKDNSAFK

SEQ ID NO: 14 - protein, 63 aa, Plasmodium falciparum
ERVANCGPWDPWTACSVTCGRGTHSRSRPSLHEKCTTHMVSECEEGECPVEPEPLPVPAPLPT

SEQ ID NO: 15 - protein, 54 aa, Plasmodium falciparum
EVERIAKCGPWDDWTPCSVTCGKGTHSRSRPLLHAGCTTHMVKECEMDECPVEP

SEQ ID NO: 16 - protein, 201 aa, Plasmodium falciparum
EKTASCGVWDEWSPCSVTCGKGTRSRKREILHEGCTSELQEQCEEERCLPKAAVAMAERVANCGPWDPWTACSVTCGRGTHSRSRPSLHEKCTTHMVSECEEGECPVEPEPLPVPAPLPTAAVAMAEVERIAKCGPTAATCGGCCGTGGCCATGGCTWDDWTPCSVTCGKGTHSRSRPLLHAGCTTHMVKECEMDECPVEP

SEQ ID NO: 17 - protein, 329 aa, Plasmodium falciparum
SKEIVKKYNLNLRNAILNNNSQIENEENVNTTITGNDFSGGEFLWPGYTEELKAKKASEDAEKAANDAENASKEAEEAAKEAVNLKESDKSYTKAKEAATAASKAKKAVETALKAKDDAEKSSKADSISTKTKEYAEKAKNAYEKAKNAYQKANQAVLKAKEASSYDYILGWEFGGGVPEHKKEENMLSHLYVSSKDKENIAKENDDVLDEKEEEAEETEEEELEEKNEEETESEISEDEEEEEEEEKEEENDKKKEQEKEQSNENNDQKKDMEAQNLISKNQNNNEKNVKEAAESIMKTLAGLIKGNNQIDSTLKDLVEELSKYFKNH

SEQ ID NO: 18 - protein, 355 aa, Plasmodium falciparum
SKEIVKKYNLNLRNAILNNNSQIENEENDIKYELNEQNDENVNTPIVGNMEFGEGFSADDQKDIEAYKKAKQASQDAEQAAKDAENAAKDAEEAAKDAEKLKESDESYTKAKEACTAASKAKKAVETALKAKDDAETALKTSETPEKPSRINLFSRKTKEYAEKAKNAYEKAKNAYQKANQAVLKAKEASSYDYILGWEFGGGVPEHKKEENMLSHLYVSSKDKENIAKENDDVLDEKEEEAEETEEEELEEKNEEETESEISEDEEEEEEEEKEEENDKKKEQEKEQSNENNDQKKDMEAQNLISKNQNNNEKNVKEAAESIMKTLAGLIKGNNQIDSTLKDLVEELSKYFKNH

SEQ ID NO: 19 - protein, 226 aa, Plasmodium falciparum
NGLTGATENIAQVVQANSETNKNPTSHSNSTTTSLNNNILGWEFGGGAPQNGAAEDKKTEYLLEQIKIPSWDRNNIPDENEQVIEDPQEDNKDEDEDEETETENLETEDDNNEEIEENEEDDIDEESVEEKEEEEEKKEEEEKKEEKKEEKKPDNEITNEVKEEQKYSSPSDINAQNLISNKNKKNDETKKTAENIVKTLVGLFNEKNEIDSTINNLVQEMIHLFS

SEQ ID NO: 20 - protein, 174 aa, Plasmodium falciparum
SETDTQSKNEQEISTQGQEVQKPAQGGESTFQKDLDKKLYNLGDVFNHVVDISNKENKINLDEHDKKYTDFKKEYEDFVLNSKEYDIIKNLIIMFGQEDNKAKNGKTDIVSEAKHITEIFIKLFKDKEYHEQFKNYIYGVYSYAKQNSHLSEKKIKQEEEYKKFLEYSFNLLNT

SEQ ID NO: 21 - protein, 118 aa, Plasmodium falciparum
EKTNKGTGSGVSSKKKNKKGSGEPLIDVHDLISDMIKKEEELVEVNKRKSKYKLATSNTEKGRHPFKIGSSDPADNANPDADSESNGEPNADPQVTAQDVTPEQPQGDDNNLVSGPEH

SEQ ID NO: 22 - protein, 283 aa, Plasmodium falciparum
DKNSVDTNTKVWECKKPYKLSTKDVCVPPRRQELCLGNIDRIYDKNLLMIKEHILAIAIYESRILKRKYKNKDDKEVCKIINKAFADIRDIIGGTDYWNDLSNRKLVGKINTNSNYVHRNKQNDKLFRDEWWKVIKKDVWNVISWVFKDKTVCKEDDIENIPQFFRWFSEWGDDYCQDKTKMIETLKVECKEKPCEDDNCKRKCNSYKEWISKKKEEYNKQAKQYQEYQKGNNYKMYSEFKSIKPEVYLKKYSEKCSNLNFEDEFKEELHSDYKNKCTMCPEV

SEQ ID NO: 23 - protein, 450 aa, Plasmodium falciparum
IEIVERSNYMGNPWTEYMAKYDIEEVHGSGIRVDLGEDAEVAGTQYRLPSGKCPVFGKGIIIENSNTAFLTPVATGNQYLKDGGFAFPPTEPLMSPMTLDEMRHFYKDNKYVKNLDELTLCSRHAGNMIPDNDKNSNYKYPAVYDDKDKKCHILYIAAQENNGPRYCNKDESKRNSMFCFRPAKDISFQNYAYLSKNVVDNWEKVCPRKNLQNAKFGLWVDGNCEDIPHVNEFPAIDLFECNKLVFELSASDQPKQYEQHLTDYEKIKEGFKNKNAAMIKSAFLPTGAFKADRYKSHGKGYNWGNYNTETQKCEIFNVKPTCLINNAAYIATTALSHPIEVENNFPCSLYKDEIMKEIERESKRIKLNDNDDEGNKKIIAPRIFISDDKDSLKCPCDPEMVSNSACRFFVCKCVERRAEVTSNNEVVVKEEYKDEYADIPEHKPTYDKMK

SEQ ID NO: 24 - protein, 48 aa, Plasmodium falciparum
MDITQQAKDIGAGPVASCFTTRMSPPQQICLNSVVNTALSTSTQSAMK

SEQ ID NO: 25 - protein, 113 aa, Plasmodium falciparum
KKYETYVDMKTIESKYTTVMTLSEHLLEYAMDVLKANPQKPIDPKANLDSEVVKLQIKINEKSNELDNAASQVKTLIIIMKSFYDIIISEKASMDEMEKKELSLNNYIEKTDY

SEQ ID NO: 26 - protein, 317 aa, Plasmodium falciparum
DSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNSEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTTAGCYGGTFVKIDENRTLFIYS

SEQ ID NO: 27 - protein, 288 aa, Plasmodium falciparum
NEDRGIYDELLENDMCDLYNLKMHDLHNLKSYDFGLSKDLLKKDIFIYSNNLKNDDMDDDDNNNMNDIAIGENVIYENDIHENNIDDNDMYNNYVNGNDLYINNMQDDAMDDIVYDEEEIKSFLDKLKSDISNQMNVKNGNVEVTGNGGNEEMSYINNDENLQAFDLLDNFHMDDYGNNYNDNEEDGDGDGDDDEQKKRKQKELHNVNGKLDLSDLNELNVDDINNNFYMSTPRKSIDERKDTECQTDFPLLDVSRNTDRTPRRKSVEVILVEAAAHHHHHHSEKDEL

SEQ ID NO: 28 - protein, 520 aa, Plasmodium falciparum
QNYWEHPYQKSDVYHPINEHREHPKEYEYPLHQEHTYQQEDSGEDENTLQHAYPIDHEGAEPAPQEQNLFSSIEIVERSNYMGNPWTEYMAKYDIEEVHGSGIRVDLGEDAEVAGTQYRLPSGKCPVFGKGIIIENSQTTFLTPVATENQDLKDGGFAFPPTKPLMSPMTLDQMRHFYKDNEYVKNLDELTLCSRHAGNMNPDNDKNSNYKYPAVYDDKDKKCHILYIAAQENNGPRYCNKDESKRNSMFCFRPAKDKSFQNYVYLSKNVVDNWEKVCPRKNLENAKFGLWVDGNCEDIPHVNEFSANDLFECNKLVFELSASDQPKQYEQHLTDYEKIKEGFKNKNADMIRSAFLPTGAFKADRYKSHGKGYNWGNYNRKTQKCEIFNVKPTCLINDKSYIATTALSHPIEVEHNFPCSLYKDEIKKEIERESKRIKLNDNDDEGNKKIIAPRIFISDDKDSLKCPCDPEIVSQSTCNFFVCKCVEKRAEVTSNNEVVVKEEYKDEYADIPEHKPTYDK

SEQ ID NO: 29 - protein, 520 aa, Plasmodium falciparum
QNYWEHPYQKSDVYHPINEHREHPKEYEYPLHQEHTYQQEDSGEDENTLQHAYPIDHEGAEPAPQEQNLFSSIEIVERSNYMGNPWTEYMAKYDIEEVHGSGIRVDLGEDAEVAGTQYRLPSGKCPVFGKGIIIENSQTTFLKPVATGNQDLKDGGFAFPPTNPLISPMTLNGMRDFYKNNEYVKNLDELTLCSRHAGNMNPDNDENSNYKYPAVYDYNDKKCHILYIAAQENNGPRYCNKDESKRNSMFCFRPAKDKLFENYVYLSKNVVHNWEEVCPRKNLENAKFGLWVDGNCEDIPHVNEFSANDLFECNKLVFELSASDQPKQYEQHLTDYEKIKEGFKNKNADMIRSAFLPTGAFKADRYKSRGKGYNWGNYNRKTQKCEIFNVKPTCLINDKSYIATTALSHPIEVENNFPCSLYKNEIMKEIERESKRIKLNDNDDEGNKKIIAPRIFISDDKDSLKCPCDPEMVSQSTCRFFVCKCVERRAEVTSNNEVVVKEEYKDEYADIPEHKPTYDN

SEQ ID NO: 30 - protein, 520 aa, Plasmodium falciparum
QNYWEHPYQKSDVYHPINEHREHPKEYEYPLHQEHTYQQEDSGEDENTLQHAYPIDHEGAEPAPQEQNLFSSIEIVERSNYMGNPWTEYMAKYDIEEVHGSGIRVDLGEDAEVAGTQYRLPSGKCPVFGKGIIIENSKTTFLTPVATENQDLKDGGFAFPPTEPLMSPMTLDDMRDLYKDNKYVKNLDELTLCSRHAGNMIPDNDKNSNYKYPAVYDYEDKKCHILYIAAQENNGPRYCNKDQSKRNSMFCFRPAKDISFQNYVYLSKNVVDNWEKVCPRKNLQNAKFGLWVDGNCEDIPHVNEFSAIDLFECNKLVFELSASDQPKQYEQHLTDYEKIKEGFKNKNADMIRSAFLPTGAFKADRYKSHGKGYNWGNYNTETQKCEIFNVKPTCLINDKSYIATTALSHPNEVEHNFPCSLYKDEIKKEIERESKRIKLNDNDDEGNKKIIAPRIFISDDIDSLKCPCAPEIVSQSTCNFFVCKCVEKRAEVTSNNEVVVKEEYKDEYADIPEHKPTYDK

SEQ ID NO: 31 - protein, 502 aa, Plasmodium falciparum
FENAIKKTKNQENNLALLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKINEAYDKVKSKCNDIKNDLIATIKKLEHPYDINNKNDDSYRYDISEEIDDKSEETDDETEEVEDSIQDTDSNHAPSNKKKNDLMNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 32 - protein, 333 aa, Homo sapiens
KGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 33 - protein, 431 aa, Artificial Sequence
  Malaria Immunoassemblin heavy chain fusion polypeptide
ISQHQCVKKQCPENSGCFRHLDEREECKCLLNYKQEGDKCVENPNPTCNENNGGCDADATCTEEDSGSSRKKITCECTKPDSYPLFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 34 - protein, 734 aa, Artificial Sequence
  Malaria Immunoassemblin heavy chain fusion polypeptide
ISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 35 - protein, 842 aa, Artificial Sequence
  MIA HC fusion
ISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHMLNISQHQCVKKQCPENSGCFRHLDEREECKCLLNYKQEGDKCVENPNPTCNENNGGCDADATCTEEDSGSSRKKITCECTKPDSYPLFDGIFCSSSNAAAHHHHHHSEKDEL

SEQ ID NO: 36 - protein, 505 aa, Artificial Sequence
  Malaria Immunoassemblin heavy chain fusion polypeptide
VTVDTVCKRGFLIQMSGHLECKCENDLVLVNEETCEEKVLKCDEKTVNKPCGDFSKCIKIDGNPVSYACKCNLGYDMVNNVCIPNECKNVACGNGKCILDTSNPVKTGVCSCNIGKVPNVQDQKCSKDGETKCSLKCLKENEACKAVDGIYKCDCKDGFIIDNEASICTAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 37 - protein, 505 aa, Artificial Sequence
  Malaria Immunoassemblin heavy chain fusion polypeptide
VTVDTVCKRGFLIQMSGHLECKCENDTVLVNEETCEEKVLKCDEKTVNKPCGDFSKCIKIDGNPVSYACKCNLGYDMVNNVCIPNECKNVTCGNGKCILDTSNPVKTGVCSCNIGKVPNVQDQKCSKDGETKCSLKCLKENETCKAVDGIYKCDCKDGFIIDNEASICTAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 38 - protein, 505 aa, Artificial Sequence
  Malaria Immunoassemblin heavy chain fusion polypeptide
VTENTICKYGYLIQMSNHYECKCIEGYVLINEDTCGKKVVCDKVENSFKACDEYAYCFDLGNKNNEKQIKCMCRTEYTLTAGVCVPNVCRDKVCGKGKCIVDPANSLTHTCSCNIGTILNQNKLCDIQGDTPCSLKCAENEVCTLEGNYYTCKEDPSSNGGGNTVDQASAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 39 - protein, 410 aa, Artificial Sequence
  Malaria Immunoassemblin heavy chain fusion polypeptide
PSDKHIEQYLKKIQNSLSTEWSPCSVTCGNGIQVRIKPGSANKPKDELDYENDIEKKICKMEKCSSVFNVVNSSAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 40 - protein, 410 aa, Artificial Sequence
  Malaria Immunoassemblin heavy chain fusion polypeptide
THDTCDEWSEWSACTHGISTRKCLSDSSIKDETLVCTKCDKWGEWSECKDGRMHRKVLNCPFIKEEQECDVNNEAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 41 - protein, 619 aa, Artificial Sequence
  Malaria Immunoassemblin heavy chain fusion polypeptide
PSDKHIEQYLKKIQNSLSTEWSPCSVTCGNGIQVRIKPGSANKPKDELDYENDIEKKICKMEKCSSVFNVVNSSAAVAMAEKTASCGVWDEWSPCSVTCGKGTRSRKREILHEGCTSELQEQCEEERCLPKAAVAMATHDTCDEWSEWSACTHGISTRKCLSDSSIKDETLVCTKCDKWGEWSECKDGRMHRKVLNCPFIKEEQECDVNNEAAVAMAFYSEWGEWSNCAMDCDHPDNVQIRERECIHPSGDCFKGDLKESRPCIIPLPPCNELFSHKDNSAFKAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 42 - protein, 665 aa, Artificial Sequence
  Malaria Immunoassemblin heavy chain fusion polypeptide
SKEIVKKYNLNLRNAILNNNSQIENEENVNTTITGNDFSGGEFLWPGYTEELKAKKASEDAEKAANDAENASKEAEEAAKEAVNLKESDKSYTKAKEAATAASKAKKAVETALKAKDDAEKSSKADSISTKTKEYAEKAKNAYEKAKNAYQKANQAVLKAKEASSYDYILGWEFGGGVPEHKKEENMLSHLYVSSKDKENIAKENDDVLDEKEEEAEETEEEELEEKNEEETESEISEDEEEEEEEEKEEENDKKKEQEKEQSNENNDQKKDMEAQNLISKNQNNNEKNVKEAAESIMKTLAGLIKGNNQIDSTLKDLVEELSKYFKNHAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 43 - protein, 691 aa, Artificial Sequence
  Malaria Immunoassemblin heavy chain fusion polypeptide
SKEIVKKYNLNLRNAILNNNSQIENEENDIKYELNEQNDENVNTPIVGNMEFGEGFSADDQKDIEAYKKAKQASQDAEQAAKDAENAAKDAEEAAKDAEKLKESDESYTKAKEACTAASKAKKAVETALKAKDDAETALKTSETPEKPSRINLFSRKTKEYAEKAKNAYEKAKNAYQKANQAVLKAKEASSYDYILGWEFGGGVPEHKKEENMLSHLYVSSKDKENIAKENDDVLDEKEEEAEETEEEELEEKNEEETESEISEDEEEEEEEEKEEENDKKKEQEKEQSNENNDQKKDMEAQNLISKNQNNNEKNVKEAAESIMKTLAGLIKGNNQIDSTLKDLVEELSKYFKNHAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 44 - protein, 568 aa, Artificial Sequence
  Malaria Immunoassemblin heavy chain fusion polypeptide
NGLTGATENIAQVVQANSETNKNPTSHSNSTTTSLNNNILGWEFGGGAPQNGAAEDKKTEYLLEQIKIPSWDRNNIPDENEQVIEDPQEDNKDEDEDEETETENLETEDDNNEEIEENEEDDIDEESVEEKEEEEEKKEEEEKKEEKKEEKKPDNEITNEVKEEQKYSSPSDINAQNLISNKNKKNDETKKTAENIVKTLVGLFNEKNEIDSTINNLVQEMIHLFSSKSRWSAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 45 - protein, 510 aa, Artificial Sequence
  Malaria Immunoassemblin heavy chain fusion polypeptide
SETDTQSKNEQEISTQGQEVQKPAQGGESTFQKDLDKKLYNLGDVFNHVVDISNKENKINLDEHDKKYTDFKKEYEDFVLNSKEYDIIKNLIIMFGQEDNKAKNGKTDIVSEAKHITEIFIKLFKDKEYHEQFKNYIYGVYSYAKQNSHLSEKKIKQEEEYKKFLEYSFNLLNTAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 46 - protein, 454 aa, Artificial Sequence
  Malaria Immunoassemblin heavy chain fusion polypeptide
EKTNKGTGSGVSSKKKNKKGSGEPLIDVHDLISDMIKKEEELVEVNKRKSKYKLATSNTEKGRHPFKIGSSDPADNANPDADSESNGEPNADPQVTAQDVTPEQPQGDDNNLVSGPEHAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 47 - protein, 619 aa, Artificial Sequence
  Malaria Immunoassemblin heavy chain fusion polypeptide
DKNSVDTNTKVWECKKPYKLSTKDVCVPPRRQELCLGNIDRIYDKNLLMIKEHILAIAIYESRILKRKYKNKDDKEVCKIINKAFADIRDIIGGTDYWNDLSNRKLVGKINTNSNYVHRNKQNDKLFRDEWWKVIKKDVWNVISWVFKDKTVCKEDDIENIPQFFRWFSEWGDDYCQDKTKMIETLKVECKEKPCEDDNCKRKCNSYKEWISKKKEEYNKQAKQYQEYQKGNNYKMYSEFKSIKPEVYLKKYSEKCSNLNFEDEFKEELHSDYKNKCTMCPEVAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 48 - protein, 786 aa, Artificial Sequence
  Malaria Immunoassemblin heavy chain fusion polypeptide
IEIVERSNYMGNPWTEYMAKYDIEEVHGSGIRVDLGEDAEVAGTQYRLPSGKCPVFGKGIIIENSNTAFLTPVATGNQYLKDGGFAFPPTEPLMSPMTLDEMRHFYKDNKYVKNLDELTLCSRHAGNMIPDNDKNSNYKYPAVYDDKDKKCHILYIAAQENNGPRYCNKDESKRNSMFCFRPAKDISFQNYAYLSKNVVDNWEKVCPRKNLQNAKFGLWVDGNCEDIPHVNEFPAIDLFECNKLVFELSASDQPKQYEQHLTDYEKIKEGFKNKNAAMIKSAFLPTGAFKADRYKSHGKGYNWGNYNTETQKCEIFNVKPTCLINNAAYIATTALSHPIEVENNFPCSLYKDEIMKEIERESKRIKLNDNDDEGNKKIIAPRIFISDDKDSLKCPCDPEMVSNSACRFFVCKCVERRAEVTSNNEVVVKEEYKDEYADIPEHKPTYDKMKAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 49 - protein, 384 aa, Artificial Sequence
  Malaria Immunoassemblin heavy chain fusion polypeptide
MDITQQAKDIGAGPVASCFTTRMSPPQQICLNSVVNTALSTSTQSAMKAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSEKDEL

SEQ ID NO: 50 - protein, 103 aa, Homo sapiens
APSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 200 aa, Artificial Sequence
  MIA-fusion polypeptides
ISQHQCVKKQCPENSGCFRHLDEREECKCLLNYKQEGDKCVENPNPTCNENNGGCDADATCTEEDSGSSRKKITCECTKPDSYPLFDGIFCSSSNAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 503 aa, Artificial Sequence
  MIA-fusion polypeptides
ISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSNAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 274 aa, Artificial Sequence
  MIA-fusion polypeptides
VTVDTVCKRGFLIQMSGHLECKCENDLVLVNEETCEEKVLKCDEKTVNKPCGDFSKCIKIDGNPVSYACKCNLGYDMVNNVCIPNECKNVACGNGKCILDTSNPVKTGVCSCNIGKVPNVQDQKCSKDGETKCSLKCLKENEACKAVDGIYKCDCKDGFIIDNEASICTAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 274 aa, Artificial Sequence
  MIA-fusion polypeptides
VTVDTVCKRGFLIQMSGHLECKCENDTVLVNEETCEEKVLKCDEKTVNKPCGDFSKCIKIDGNPVSYACKCNLGYDMVNNVCIPNECKNVTCGNGKCILDTSNPVKTGVCSCNIGKVPNVQDQKCSKDGETKCSLKCLKENETCKAVDGIYKCDCKDGFIIDNEASICTAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 274 aa, Artificial Sequence
  MIA-fusion polypeptides
VTENTICKYGYLIQMSNHYECKCIEGYVLINEDTCGKKVVCDKVENSFKACDEYAYCFDLGNKNNEKQIKCMCRTEYTLTAGVCVPNVCRDKVCGKGKCIVDPANSLTHTCSCNIGTILNQNKLCDIQGDTPCSLKCAENEVCTLEGNYYTCKEDPSSNGGGNTVDQASAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 179 aa, Artificial Sequence
  MIA-fusion polypeptides
PSDKHIEQYLKKIQNSLSTEWSPCSVTCGNGIQVRIKPGSANKPKDELDYENDIEKKICKMEKCSSVFNVVNSSAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 179 aa, Artificial Sequence
  MIA-fusion polypeptides
THDTCDEWSEWSACTHGISTRKCLSDSSIKDETLVCTKCDKWGEWSECKDGRMHRKVLNCPFIKEEQECDVNNEAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 388 aa, Artificial Sequence
  MIA-fusion polypeptides
PSDKHIEQYLKKIQNSLSTEWSPCSVTCGNGIQVRIKPGSANKPKDELDYENDIEKKICKMEKCSSVFNVVNSSAAVAMAEKTASCGVWDEWSPCSVTCGKGTRSRKREILHEGCTSELQEQCEEERCLPKAAVAMATHDTCDEWSEWSACTHGISTRKCLSDSSIKDETLVCTKCDKWGEWSECKDGRMHRKVLNCPFIKEEQECDVNNEAAVAMAFYSEWGEWSNCAMDCDHPDNVQIRERECIHPSGDCFKGDLKESRPCIIPLPPCNELFSHKDNSAFKAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 434 aa, Artificial Sequence
  MIA-fusion polypeptides
SKEIVKKYNLNLRNAILNNNSQIENEENVNTTITGNDFSGGEFLWPGYTEELKAKKASEDAEKAANDAENASKEAEEAAKEAVNLKESDKSYTKAKEAATAASKAKKAVETALKAKDDAEKSSKADSISTKTKEYAEKAKNAYEKAKNAYQKANQAVLKAKEASSYDYILGWEFGGGVPEHKKEENMLSHLYVSSKDKENIAKENDDVLDEKEEEAEETEEEELEEKNEEETESEISEDEEEEEEEEKEEENDKKKEQEKEQSNENNDQKKDMEAQNLISKNQNNNEKNVKEAAESIMKTLAGLIKGNNQIDSTLKDLVEELSKYFKNHAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 460 aa, Artificial Sequence
  MIA-fusion polypeptides
SKEIVKKYNLNLRNAILNNNSQIENEENDIKYELNEQNDENVNTPIVGNMEFGEGFSADDQKDIEAYKKAKQASQDAEQAAKDAENAAKDAEEAAKDAEKLKESDESYTKAKEACTAASKAKKAVETALKAKDDAETALKTSETPEKPSRINLFSRKTKEYAEKAKNAYEKAKNAYQKANQAVLKAKEASSYDYILGWEFGGGVPEHKKEENMLSHLYVSSKDKENIAKENDDVLDEKEEEAEETEEEELEEKNEEETESEISEDEEEEEEEEKEEENDKKKEQEKEQSNENNDQKKDMEAQNLISKNQNNNEKNVKEAAESIMKTLAGLIKGNNQIDSTLKDLVEELSKYFKNHAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 337 aa, Artificial Sequence
  MIA-fusion polypeptides
NGLTGATENIAQVVQANSETNKNPTSHSNSTTTSLNNNILGWEFGGGAPQNGAAEDKKTEYLLEQIKIPSWDRNNIPDENEQVIEDPQEDNKDEDEDEETETENLETEDDNNEEIEENEEDDIDEESVEEKEEEEEKKEEEEKKEEKKEEKKPDNEITNEVKEEQKYSSPSDINAQNLISNKNKKNDETKKTAENIVKTLVGLFNEKNEIDSTINNLVQEMIHLFSSKSRWSAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 279 aa, Artificial Sequence
  MIA-fusion polypeptides
SETDTQSKNEQEISTQGQEVQKPAQGGESTFQKDLDKKLYNLGDVFNHVVDISNKENKINLDEHDKKYTDFKKEYEDFVLNSKEYDIIKNLIIMFGQEDNKAKNGKTDIVSEAKHITEIFIKLFKDKEYHEQFKNYIYGVYSYAKQNSHLSEKKIKQEEEYKKFLEYSFNLLNTAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 223 aa, Artificial Sequence
  MIA-fusion polypeptides
EKTNKGTGSGVSSKKKNKKGSGEPLIDVHDLISDMIKKEEELVEVNKRKSKYKLATSNTEKGRHPFKIGSSDPADNANPDADSESNGEPNADPQVTAQDVTPEQPQGDDNNLVSGPEHAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 388 aa, Artificial Sequence
  MIA-fusion polypeptides
DKNSVDTNTKVWECKKPYKLSTKDVCVPPRRQELCLGNIDRIYDKNLLMIKEHILAIAIYESRILKRKYKNKDDKEVCKIINKAFADIRDIIGGTDYWNDLSNRKLVGKINTNSNYVHRNKQNDKLFRDEWWKVIKKDVWNVISWVFKDKTVCKEDDIENIPQFFRWFSEWGDDYCQDKTKMIETLKVECKEKPCEDDNCKRKCNSYKEWISKKKEEYNKQAKQYQEYQKGNNYKMYSEFKSIKPEVYLKKYSEKCSNLNFEDEFKEELHSDYKNKCTMCPEVAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 555 aa, Artificial Sequence
  MIA-fusion polypeptides
IEIVERSNYMGNPWTEYMAKYDIEEVHGSGIRVDLGEDAEVAGTQYRLPSGKCPVFGKGIIIENSNTAFLTPVATGNQYLKDGGFAFPPTEPLMSPMTLDEMRHFYKDNKYVKNLDELTLCSRHAGNMIPDNDKNSNYKYPAVYDDKDKKCHILYIAAQENNGPRYCNKDESKRNSMFCFRPAKDISFQNYAYLSKNVVDNWEKVCPRKNLQNAKFGLWVDGNCEDIPHVNEFPAIDLFECNKLVFELSASDQPKQYEQHLTDYEKIKEGFKNKNAAMIKSAFLPTGAFKADRYKSHGKGYNWGNYNTETQKCEIFNVKPTCLINNAAYIATTALSHPIEVENNFPCSLYKDEIMKEIERESKRIKLNDNDDEGNKKIIAPRIFISDDKDSLKCPCDPEMVSNSACRFFVCKCVERRAEVTSNNEVVVKEEYKDEYADIPEHKPTYDKMKAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 153 aa, Artificial Sequence
  MIA LC fusions
MDITQQAKDIGAGPVASCFTTRMSPPQQICLNSVVNTALSTSTQSAMKAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 914 aa, Artificial Sequence
  MIAs comprising an additional C-terminal fusion polypeptide
ISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHMVTENTICKYGYLIQMSNHYECKCIEGYVLINEDTCGKKVVCDKVENSFKACDEYAYCFDLGNKNNEKQIKCMCRTEYTLTAGVCVPNVCRDKVCGKGKCIVDPANSLTHTCSCNIGTILNQNKLCDIQGDTPCSLKCAENEVCTLEGNYYTCKEDPSSNGGGNTVDQASAAAHHHHHHSEKDEL

SEQ ID NO: 68 - protein, 820 aa, Artificial Sequence
  MIAs comprising an additional C-terminal fusion polypeptide
ISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHMAPSDKHIEQYLKKIQNSLSTEWSPCSVTCGNGIQVRIKPGSANKPKDELDYENDIEKKICKMEKCSSVFNVVNSSAAAHHHHHHSEKDEL

SEQ ID NO: 69 - protein, 1089 aa, Artificial Sequence
  MIAs comprising an additional C-terminal fusion polypeptide
ISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHMVAAGDLRISSKEIVKKYNLNLRNAILNNNSQIENEENVNTTITGNDFSGGEFLWPGYTEELKAKKASEDAEKAANDAENASKEAEEAAKEAVNLKESDKSYTKAKEAATAASKAKKAVETALKAKDDAEKSSKADSISTKTKEYAEKAKNAYEKAKNAYQKANQAVLKAKEASSYDYILGWEFGGGVPEHKKEENMLSHLYVSSKDKENIAKENDDVLDEKEEEAEETEEEELEEKNEEETESEISEDEEEEEEEEKEEENDKKKEQEKEQSNENNDQKKDMEAQNLISKNQNNNEKNVKEAAESIMKTLAGLIKGNNQIDSTLKDLVEELSKYFKNHSKSRWSAAAHHHHHHSEKDEL

SEQ ID NO: 70 - protein, 1117 aa, Artificial Sequence
  MIAs comprising an additional C-terminal fusion polypeptide
MAISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHMVAAGDLRISSKEIVKKYNLNLRNAILNNNSQIENEENDIKYELNEQNDENVNTPIVGNMEFGEGFSADDQKDIEAYKKAKQASQDAEQAAKDAENAAKDAEEAAKDAEKLKESDESYTKAKEACTAASKAKKAVETALKAKDDAETALKTSETPEKPSRINLFSRKTKEYAEKAKNAYEKAKNAYQKANQAVLKAKEASSYDYILGWEFGGGVPEHKKEENMLSHLYVSSKDKENIAKENDDVLDEKEEEAEETEEEELEEKNEEETESEISEDEEEEEEEEKEEENDKKKEQEKEQSNENNDQKKDMEAQNLISKNQNNNEKNVKEAAESIMKTLAGLIKGNNQIDSTLKDLVEELSKYFKNHSKSRWSAAAHHHHHHSEKDEL

SEQ ID NO: 71 - protein, 986 aa, Artificial Sequence
  MIAs comprising an additional C-terminal fusion polypeptide
ISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHMVAAGDLRISNGLTGATENIAQVVQANSETNKNPTSHSNSTTTSLNNNILGWEFGGGAPQNGAAEDKKTEYLLEQIKIPSWDRNNIPDENEQVIEDPQEDNKDEDEDEETETENLETEDDNNEEIEENEEDDIDEESVEEKEEEEEKKEEEEKKEEKKEEKKPDNEITNEVKEEQKYSSPSDINAQNLISNKNKKNDETKKTAENIVKTLVGLFNEKNEIDSTINNLVQEMIHLFSSKSRWSAAAHHHHHHSEKDEL

SEQ ID NO: 72 - protein, 920 aa, Artificial Sequence
  MIAs comprising an additional C-terminal fusion polypeptide
ISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHMGSETDTQSKNEQEISTQGQEVQKPAQGGESTFQKDLDKKLYNLGDVFNHVVDISNKENKINLDEHDKKYTDFKKEYEDFVLNSKEYDIIKNLIIMFGQEDNKAKNGKTDIVSEAKHITEIFIKLFKDKEYHEQFKNYIYGVYSYAKQNSHLSEKKIKQEEEYKKFLEYSFNLLNTAAAHHHHHHSEKDEL

SEQ ID NO: 73 - protein, 540 aa, Artificial Sequence
  MIAs comprising an additional C-terminal fusion polypeptide
THDTCDEWSEWSACTHGISTRKCLSDSSIKDETLVCTKCDKWGEWSECKDGRMHRKVLNCPFIKEEQECDVNNEAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHMAEKTNKGTGSGVSSKKKNKKGSGEPLIDVHDLISDMIKKEEELVEVNKRKSKYKLATSNTEKGRHPFKIGSSDPADNANPDADSESNGEPNADPQVTAQDVTPEQPQGDDNNLVSGPEHAAAHHHHHHSEKDEL

SEQ ID NO: 74 - protein, 1029 aa, Artificial Sequence
  MIAs comprising an additional C-terminal fusion polypeptide
ISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHMADKNSVDTNTKVWECKKPYKLSTKDVCVPPRRQELCLGNIDRIYDKNLLMIKEHILAIAIYESRILKRKYKNKDDKEVCKIINKAFADIRDIIGGTDYWNDLSNRKLVGKINTNSNYVHRNKQNDKLFRDEWWKVIKKDVWNVISWVFKDKTVCKEDDIENIPQFFRWFSEWGDDYCQDKTKMIETLKVECKEKPCEDDNCKRKCNSYKEWISKKKEEYNKQAKQYQEYQKGNNYKMYSEFKSIKPEVYLKKYSEKCSNLNFEDEFKEELHSDYKNKCTMCPEVAAAHHHHHHSEKDEL

SEQ ID NO: 75 - protein, 792 aa, Artificial Sequence
  MIAs comprising an additional C-terminal fusion polypeptide
ISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHMDITQQAKDIGAGPVASCFTTRMSPPQQICLNSVVNTALSTSTQSAMKAAAHHHHHHSEKDEL

SEQ ID NO: 76 - protein, 332 aa, Artificial Sequence
  MIAs with modified CH3 regions for Fc-heterodimerization
KGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDKLTKNQVSLTCLVKGFYPSDIAVWESNGQPENNYKTTPPVLKSDGSFFLYSKLTVDKSRWQQGNVFSCSVMHEALHNHYTQKSLSLSPGKSEKDEL

SEQ ID NO: 77 - protein, 332 aa, Artificial Sequence
  MIAs with modified CH3 regions for Fc-heterodimerization
KGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVWESNGQPENNYDTTPPVLDSDGSFFLYSDLTVDKSRWQQGNVFSCSVMHEALHNHYTQKSLSLSPGKSEKDEL

SEQ ID NO: 78 - protein, 431 aa, Artificial Sequence
  MIAs with modified CH3 regions for Fc-heterodimerization
ISQHQCVKKQCPENSGCFRHLDEREECKCLLNYKQEGDKCVENPNPTCNENNGGCDADATCTEEDSGSSRKKITCECTKPDSYPLFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGKSEKDEL

SEQ ID NO: 79 - protein, 431 aa, Artificial Sequence
  MIAs with modified CH3 regions for Fc-heterodimerization
ISQHQCVKKQCPENSGCFRHLDEREECKCLLNYKQEGDKCVENPNPTCNENNGGCDADATCTEEDSGSSRKKITCECTKPDSYPLFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKSEKDEL

SEQ ID NO: 80 - protein, 505 aa, Artificial Sequence
  MIAs with modified CH3 regions for Fc-heterodimerization
VTVDTVCKRGFLIQMSGHLECKCENDTVLVNEETCEEKVLKCDEKTVNKPCGDFSKCIKIDGNPVSYACKCNLGYDMVNNVCIPNECKNVTCGNGKCILDTSNPVKTGVCSCNIGKVPNVQDQKCSKDGETKCSLKCLKENETCKAVDGIYKCDCKDGFIIDNEASICTAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGKSEKDEL

SEQ ID NO: 81 - protein, 505 aa, Artificial Sequence
  MIAs with modified CH3 regions for Fc-heterodimerization
VTVDTVCKRGFLIQMSGHLECKCENDTVLVNEETCEEKVLKCDEKTVNKPCGDFSKCIKIDGNPVSYACKCNLGYDMVNNVCIPNECKNVTCGNGKCILDTSNPVKTGVCSCNIGKVPNVQDQKCSKDGETKCSLKCLKENETCKAVDGIYKCDCKDGFIIDNEASICTAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKSEKDEL

SEQ ID NO: 82 - protein, 410 aa, Artificial Sequence
  MIAs with modified CH3 regions for Fc-heterodimerization
THDTCDEWSEWSACTHGISTRKCLSDSSIKDETLVCTKCDKWGEWSECKDGRMHRKVLNCPFIKEEQECDVNNEAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGKSEKDEL

SEQ ID NO: 83 - protein, 410 aa, Artificial Sequence
  MIAs with modified CH3 regions for Fc-heterodimerization
THDTCDEWSEWSACTHGISTRKCLSDSSIKDETLVCTKCDKWGEWSECKDGRMHRKVLNCPFIKEEQECDVNNEAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKSEKDEL

SEQ ID NO: 84 - protein, 786 aa, Artificial Sequence
  MIAs with modified CH3 regions for Fc-heterodimerization
IEIVERSNYMGNPWTEYMAKYDIEEVHGSGIRVDLGEDAEVAGTQYRLPSGKCPVFGKGIIIENSNTAFLTPVATGNQYLKDGGFAFPPTEPLMSPMTLDEMRHFYKDNKYVKNLDELTLCSRHAGNMIPDNDKNSNYKYPAVYDDKDKKCHILYIAAQENNGPRYCNKDESKRNSMFCFRPAKDISFQNYAYLSKNVVDNWEKVCPRKNLQNAKFGLWVDGNCEDIPHVNEFPAIDLFECNKLVFELSASDQPKQYEQHLTDYEKIKEGFKNKNAAMIKSAFLPTGAFKADRYKSHGKGYNWGNYNTETQKCEIFNVKPTCLINNAAYIATTALSHPIEVENNFPCSLYKDEIMKEIERESKRIKLNDNDDEGNKKIIAPRIFISDDKDSLKCPCDPEMVSNSACRFFVCKCVERRAEVTSNNEVVVKEEYKDEYADIPEHKPTYDKMKAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGKSEKDEL

SEQ ID NO: 85 - protein, 786 aa, Artificial Sequence
  MIAs with modified CH3 regions for Fc-heterodimerization
IEIVERSNYMGNPWTEYMAKYDIEEVHGSGIRVDLGEDAEVAGTQYRLPSGKCPVFGKGIIIENSNTAFLTPVATGNQYLKDGGFAFPPTEPLMSPMTLDEMRHFYKDNKYVKNLDELTLCSRHAGNMIPDNDKNSNYKYPAVYDDKDKKCHILYIAAQENNGPRYCNKDESKRNSMFCFRPAKDISFQNYAYLSKNVVDNWEKVCPRKNLQNAKFGLWVDGNCEDIPHVNEFPAIDLFECNKLVFELSASDQPKQYEQHLTDYEKIKEGFKNKNAAMIKSAFLPTGAFKADRYKSHGKGYNWGNYNTETQKCEIFNVKPTCLINNAAYIATTALSHPIEVENNFPCSLYKDEIMKEIERESKRIKLNDNDDEGNKKIIAPRIFISDDKDSLKCPCDPEMVSNSACRFFVCKCVERRAEVTSNNEVVVKEEYKDEYADIPEHKPTYDKMKAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKSEKDEL

SEQ ID NO: 86 - protein, 113 aa, Artificial Sequence; identical to SEQ ID NO: 25
  MIAs with modified CH3 regions for Fc-heterodimerization

SEQ ID NO: 87 - protein, 449 aa, Artificial Sequence
  MIAs with modified CH3 regions for Fc-heterodimerization
KKYETYVDMKTIESKYTTVMTLSEHLLEYAMDVLKANPQKPIDPKANLDSEVVKLQIKINEKSNELDNAASQVKTLIIIMKSFYDIIISEKASMDEMEKKELSLNNYIEKTDYAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGKSEKDEL

SEQ ID NO: 88 - protein, 449 aa, Artificial Sequence
  MIAs with modified CH3 regions for Fc-heterodimerization
KKYETYVDMKTIESKYTTVMTLSEHLLEYAMDVLKANPQKPIDPKANLDSEVVKLQIKINEKSNELDNAASQVKTLIIIMKSFYDIIISEKASMDEMEKKELSLNNYIEKTDYAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKSEKDEL

SEQ ID NO: 89 - protein, 653 aa, Artificial Sequence
  MIAs with modified CH3 regions for Fc-heterodimerization
DSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNSEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTTAGCYGGTFVKIDENRTLFIYSAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGKSEKDEL

SEQ ID NO: 90 - protein, 653 aa, Artificial Sequence
  MIAs with modified CH3 regions for Fc-heterodimerization
DSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNSEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTTAGCYGGTFVKIDENRTLFIYSAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKSEKDEL

SEQ ID NO: 91 - protein, 609 aa, Artificial Sequence
  MIAs with modified CH3 regions for Fc-heterodimerization
NEDRGIYDELLENDMCDLYNLKMHDLHNLKSYDFGLSKDLLKKDIFIYSNNLKNDDMDDDDNNNMNDIAIGENVIYENDIHENNIDDNDMYNNYVNGNDLYINNMQDDAMDDIVYDEEEIKSFLDKLKSDISNQMNVKNGNVEVTGNGGNEEMSYINNDENLQAFDLLDNFHMDDYGNNYNDNEEDGDGDGDDDEQKKRKQKELHNVNGKLDLSDLNELNVDDINNNFYMSTPRKSIDERKDTECQTDFPLLDVSRNTDRTPRRKSVEVILVEAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGKSEKDEL

SEQ ID NO: 92 - protein, 609 aa, Artificial Sequence
  MIAs with modified CH3 regions for Fc-heterodimerization
NEDRGIYDELLENDMCDLYNLKMHDLHNLKSYDFGLSKDLLKKDIFIYSNNLKNDDMDDDDNNNMNDIAIGENVIYENDIHENNIDDNDMYNNYVNGNDLYINNMQDDAMDDIVYDEEEIKSFLDKLKSDISNQMNVKNGNVEVTGNGGNEEMSYINNDENLQAFDLLDNFHMDDYGNNYNDNEEDGDGDGDDDEQKKRKQKELHNVNGKLDLSDLNELNVDDINNNFYMSTPRKSIDERKDTECQTDFPLLDVSRNTDRTPRRKSVEVILVEAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKSEKDEL

SEQ ID NO: 93 - protein, 820 aa, Artificial Sequence
  MIA-C fusion polypeptides
ISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGKHMAPSDKHIEQYLKKIQNSLSTEWSPCSVTCGNGIQVRIKPGSANKPKDELDYENDIEKKICKMEKCSSVFNVVNSSAAAHHHHHHSEKDEL

SEQ ID NO: 94 - protein, 820 aa, Artificial Sequence
  MIA-C fusion polypeptides
ISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKHMAPSDKHIEQYLKKIQNSLSTEWSPCSVTCGNGIQVRIKPGSANKPKDELDYENDIEKKICKMEKCSSVFNVVNSSAAAHHHHHHSEKDEL

SEQ ID NO: 95 - protein, 792 aa, Artificial Sequence
  MIA-C fusion polypeptides
ISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGKHMDITQQAKDIGAGPVASCFTTRMSPPQQICLNSVVNTALSTSTQSAMKAAAHHHHHHSEKDEL

SEQ ID NO: 96 - protein, 792 aa, Artificial Sequence
  MIA-C fusion polypeptides
ISQHQCVKKQCPENSGCFRHLDEREECKCLLNYKQEGDKCVENPNPTCNENNGGCDADATCTEEDSGSSRKKITCECTKPDSYPLFDGIFCSSSNAAVAMAISQHQCVKKQCPENSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPLFDGIFCSSSNAAVAMAISQHQCVKKQCPQNSGCFRHLDEREECKCLLNYKQEGDKCVENPNPTCNENNGGCDADAKCTEEDSGSNGKKITCECTKPDSYPFFDGIFCSSSNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKHMDITQQAKDIGAGPVASCFTTRMSPPQQICLNSVVNTALSTSTQSAMKAAAHHHHHHSEKDEL

SEQ ID NO: 97 - protein, 872 aa, Artificial Sequence
  MIA-C fusion polypeptides
IEIVERSNYMGNPWTEYMAKYDIEEVHGSGIRVDLGEDAEVAGTQYRLPSGKCPVFGKGIIIENSNTAFLTPVATGNQYLKDGGFAFPPTEPLMSPMTLDEMRHFYKDNKYVKNLDELTLCSRHAGNMIPDNDKNSNYKYPAVYDDKDKKCHILYIAAQENNGPRYCNKDESKRNSMFCFRPAKDISFQNYAYLSKNVVDNWEKVCPRKNLQNAKFGLWVDGNCEDIPHVNEFPAIDLFECNKLVFELSASDQPKQYEQHLTDYEKIKEGFKNKNAAMIKSAFLPTGAFKADRYKSHGKGYNWGNYNTETQKCEIFNVKPTCLINNAAYIATTALSHPIEVENNFPCSLYKDEIMKEIERESKRIKLNDNDDEGNKKIIAPRIFISDDKDSLKCPCDPEMVSNSACRFFVCKCVERRAEVTSNNEVVVKEEYKDEYADIPEHKPTYDKMKAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGKHMAPSDKHIEQYLKKIQNSLSTEWSPCSVTCGNGIQVRIKPGSANKPKDELDYENDIEKKICKMEKCSSVFNVVNSSAAAHHHHHHSEKDEL

SEQ ID NO: 98 - protein, 872 aa, Artificial Sequence
  MIA-C fusion polypeptides
IEIVERSNYMGNPWTEYMAKYDIEEVHGSGIRVDLGEDAEVAGTQYRLPSGKCPVFGKGIIIENSNTAFLTPVATGNQYLKDGGFAFPPTEPLMSPMTLDEMRHFYKDNKYVKNLDELTLCSRHAGNMIPDNDKNSNYKYPAVYDDKDKKCHILYIAAQENNGPRYCNKDESKRNSMFCFRPAKDISFQNYAYLSKNVVDNWEKVCPRKNLQNAKFGLWVDGNCEDIPHVNEFPAIDLFECNKLVFELSASDQPKQYEQHLTDYEKIKEGFKNKNAAMIKSAFLPTGAFKADRYKSHGKGYNWGNYNTETQKCEIFNVKPTCLINNAAYIATTALSHPIEVENNFPCSLYKDEIMKEIERESKRIKLNDNDDEGNKKIIAPRIFISDDKDSLKCPCDPEMVSNSACRFFVCKCVERRAEVTSNNEVVVKEEYKDEYADIPEHKPTYDKMKAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKHMAPSDKHIEQYLKKIQNSLSTEWSPCSVTCGNGIQVRIKPGSANKPKDELDYENDIEKKICKMEKCSSVFNVVNSSAAAHHHHHHSEKDEL

SEQ ID NO: 99 - protein, 564 aa, Artificial Sequence
  MIA-C fusion polypeptides
KKYETYVDMKTIESKYTTVMTLSEHLLEYAMDVLKANPQKPIDPKANLDSEVVKLQIKINEKSNELDNAASQVKTLIIIMKSFYDIIISEKASMDEMEKKELSLNNYIEKTDYAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKHMAEKTNKGTGSGVSSKKKNKKGSGEPLIDVHDLISDMIKKEEELVEVNKRKSKYKLATSNTEKGRHPFKIGSSDPADNANPDADSESNGEPNADPQVTAQDVTPEQPQGDDNNLVSGPEH

SEQ ID NO: 100 - protein, 507 aa, Artificial Sequence
  MIA-C fusion polypeptides
KKYETYVDMKTIESKYTTVMTLSEHLLEYAMDVLKANPQKPIDPKANLDSEVVKLQIKINEKSNELDNAASQVKTLIIIMKSFYDIIISEKASMDEMEKKELSLNNYIEKTDYAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKHMDITQQAKDIGAGPVASCFTTRMSPPQQICLNSVVNTALSTSTQSAMKAAAHHHHHHSEKDEL

SEQ ID NO: 101 - protein, 739 aa, Artificial Sequence
  MIA-C fusion polypeptides
DSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNSEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTTAGCYGGTFVKIDENRTLFIYSAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGKHMAPSDKHIEQYLKKIQNSLSTEWSPCSVTCGNGIQVRIKPGSANKPKDELDYENDIEKKICKMEKCSSVFNVVNSSAAAHHHHHHSEKDEL

SEQ ID NO: 102 - protein, 711 aa, Artificial Sequence
  MIA-C fusion polypeptides
DSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNSEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTTAGCYGGTFVKIDENRTLFIYSAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKHMDITQQAKDIGAGPVASCFTTRMSPPQQICLNSVVNTALSTSTQSAMKAAAHHHHHHSEKDEL

SEQ ID NO: 103 - protein, 695 aa, Artificial Sequence
  MIA-C fusion polypeptides
NEDRGIYDELLENDMCDLYNLKMHDLHNLKSYDFGLSKDLLKKDIFIYSNNLKNDDMDDDDNNNMNDIAIGENVIYENDIHENNIDDNDMYNNYVNGNDLYINNMQDDAMDDIVYDEEEIKSFLDKLKSDISNQMNVKNGNVEVTGNGGNEEMSYINNDENLQAFDLLDNFHMDDYGNNYNDNEEDGDGDGDDDEQKKRKQKELHNVNGKLDLSDLNELNVDDINNNFYMSTPRKSIDERKDTECQTDFPLLDVSRNTDRTPRRKSVEVILVEAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGKHMAPSDKHIEQYLKKIQNSLSTEWSPCSVTCGNGIQVRIKPGSANKPKDELDYENDIEKKICKMEKCSSVFNVVNSSAAAHHHHHHSEKDEL

SEQ ID NO: 104 - protein, 667 aa, Artificial Sequence
  MIA-C fusion polypeptides
NEDRGIYDELLENDMCDLYNLKMHDLHNLKSYDFGLSKDLLKKDIFIYSNNLKNDDMDDDDNNNMNDIAIGENVIYENDIHENNIDDNDMYNNYVNGNDLYINNMQDDAMDDIVYDEEEIKSFLDKLKSDISNQMNVKNGNVEVTGNGGNEEMSYINNDENLQAFDLLDNFHMDDYGNNYNDNEEDGDGDGDDDEQKKRKQKELHNVNGKLDLSDLNELNVDDINNNFYMSTPRKSIDERKDTECQTDFPLLDVSRNTDRTPRRKSVEVILVEAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKHMDITQQAKDIGAGPVASCFTTRMSPPQQICLNSVVNTALSTSTQSAMKAAAHHHHHHSEKDEL

SEQ ID NO: 105 - protein, 541 aa, Mycobacterium tuberculosis
APKTYCQELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGKSEKDEL

SEQ ID NO: 106 - protein, 363 aa, Hepatitis C virus
QTTVVGGSQSHTVRGLTSLFSPGASQNAAAKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKSEKDEL

SEQ ID NO: 107 - protein, 513 aa, Artificial Sequence
  malaria (CLCT) and HIV (eldkwa)
FRGNNGHDSSSSLYGGSQFIEQLDNSFTSAFLESQSMNKIGDDLAETISNELVSVLQKNSPTFLESSFDIKSEVKKHAKSMLKELIKVGLPSFENLVAENVKPPKVDPATYGIIVPVLTSLFNKVETAVGAKVSDEIWNYNSPDVSESEESLSDDFFDAAGPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIEQYLKKIQNSLSTEWSPCSVTCGNGIQVRIKPGSANKPKDELDYENDIEKKICKMEKCSSVFNVVNSAAVAMAEKTASCGVWDEWSPCSVTCGKGTRSRKREILHEGCTSELQEQCEEERCLPKAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECELDKWA

(19) United States
(12) Patent Application Publication
(10) Pub. No.: US 2018/0311326 A1
(43) Pub. Date: Nov. 1, 2018

(54) IMMUNOASSEMBLIN (IA) PROTEIN COMPLEXES

(71) Applicants: Fraunhofer-Gesellschaft zur Foerderung der angewandten Forschung e.V., Munich (DE); Rheinisch-Westfaelische Technische Hochschule Aachen, Aachen (DE)

(72) Inventors: Gueven Edgue, Aachen (DE); Markus Sack, Alsdorf (DE); Veronique Beiss, Aachen (DE); Alexander Boes, Aachen (DE); Holger Spiegel, Aachen (DE); Andreas Reimann, Meerbusch (DE); Rainer Fischer, Aachen (DE)

(21) Appl. No.: 15/771,837

(22) PCT Filed: Nov. 9, 2016
(86) PCT No.: PCT/EP2016/077098

§ 371 Date: Apr. 27, 2018

(30) Foreign Application Priority Data
Nov. 10, 2015 (EP) PCT/EP2015/076194

(51) Int. Cl. A61K 39/002; C07K 14/445; C07K 16/20; A61K 39/29; C07K 16/10

(52) U.S. Cl. CPC A61K 39/002; C07K 14/445; C07K 16/205; A61K 2039/505; C07K 16/1045; C07K 16/1081; A61K 39/29

(57) ABSTRACT

The technology provided herein relates to a novel type of recombinant protein complexes, in particular of antibody-like recombinant fusion protein complexes, hereinafter referred to as immunoassemblins (IAs), suitable as animal and human vaccines comprising a plurality of antigens or antigen domains derived from proteins preferably, but not necessarily presented on the surface of a pathogen. Nucleic acid molecules encoding said recombinant proteins, vectors, host cells containing the nucleic acids and methods for preparation and producing such proteins and protein complexes; antibodies induced or generated by the use of said vaccines or said nucleic acid molecules encoding said fusion proteins and the use of such antibodies or recombinant derivatives for passive immunotherapy.

### FIELD OF THE DISCLOSURE

The present disclosure relates to a novel type of recombinant protein complexes, in particular of antibody-like recombinant fusion protein complexes, hereinafter referred to as immunoassemblins (IAs), suitable as animal and human vaccines comprising a plurality of antigens or antigen domains derived from proteins preferably, but not necessarily presented on the surface of a pathogen. Nucleic acid molecules encoding said recombinant proteins, vectors, host cells containing the nucleic acids and methods for preparation and producing such proteins and protein complexes; antibodies induced or generated by the use of said vaccines or said nucleic acid molecules encoding said fusion proteins and the use of such antibodies or recombinant derivatives for passive immunotherapy.

### BACKGROUND

An essential function of the immune system is the defense against infection. The humoral immune system combats molecules recognized as non-self, such as pathogens, using antibodies, that are raised specifically against the infectious agent, which acts as an antigen, upon first contact. Natural Antibodies are multivalent molecules comprising heavy (H) chains and light (L) chains typically joined with interchain disulfide bonds. Several isotypes of antibodies are known, including IgG1, IgG2, IgG3, IgG4, IgA, IgD, IgE, and IgM in humans. An IgG contains two heavy and two light chains. Each chain contains constant (C) and variable (V) regions, which can be further broken down into domains designated C_{H}1, C_{H}2, C_{H}3, V_{H}, and C_{L}, V_{L} (FIG. 3). Antibody binds to antigen via the variable region domains contained in the Fab portion, and after binding can interact with molecules and cells of the immune system through the constant domains, mostly through the Fc portion.

Prophylactic immunization of human and animals against microbes comprises administration of an antigen derived from the microbe in conjunction with a material that increases the antibody and/or cell-mediated immune response of the antigen in the human or animal. However, some of the difficulties still existing in today's vaccine development against several new and neglected diseases (e.g. MERS, Ebola, HIV, rabies, Malaria, Influenza) and effective counter-measures against them. A number of infectious agents are characterized by highly variable and diverse antigens (e.g. HIV, Influenza, Plasmodium ssp. and other members of the Apicomplexa), resulting in huge challenges for vaccine development. In some cases, very special and rare broadly neutralizing antibodies (bnAbs) have been identified and there are hopes that it may be possible to define and develop an antigenic counterpart that will be capable to induce such bnAbs efficiently in humans. Coverage of circulating strains of the pathogens, of escape mutants and variants emerging in the field is critical and often limits the use of a vaccine or vaccine candidate to such an extend that clinical development and market introduction is prevented.

In addition, those skilled in the field know that both the quality and quantity of the immune response induced by a vaccine depends not only on the antigenic molecules themselves but also on the formulation and in particular on the adjuvants used. The potency of a vaccine significantly depends on the formulation and the adjuvants used. Without adjuvants, the induced immune response at a given dose is weaker, which not only would increase the costs of the vaccine due to the need for higher doses, but also increase the risk for side effects. In addition, the adjuvants also influences the quality of the immune response, which is critical to provide protection. While some pathogens, e.g. rabies virus, are efficiently neutralized by a humoral response, others depend of efficient induction of T-cell responses. Although several adjuvants, especially Alumn have been successfully used for decades, there is a huge need for novel adjuvants with different modes of action. Finally, there is also a need to combine vaccines to reduce costs and the number of injections, while still providing high efficacy and population coverage.

When combining several antigens and vaccines into a single vaccine product or when giving several vaccines simultaneously, it is very important to ensure that the immune responses are directed against all components. Domination of the induced response by a single or few components would render other components essentially inactive and thus useless. Also, for any complex vaccine comprising multiple components, production costs can quickly become a limiting factor, especially if different processes for expression and purification have to be used.

Malaria for example is a disease caused by infection with protozoan parasites of the phylum Apicomplexa, namely parasites of the genus Plasmodium, globally causing more than 200 million new infections and 700 thousand deaths every year. Malaria is especially a serious problem in Africa, where one in every five (20%) childhood deaths is due to the effects of the disease. An African child has on average between 1.6 and 5.4 episodes of malaria fever each year.

Major obstacles to develop an efficient malaria vaccine result from the multi-stage life cycle of the parasite. Each stage of the parasite development is characterized by different sets of surface antigens, eliciting different types of immune responses. Despite the large variety of displayed surface antigens, the immune response against them is often ineffective. One of the reasons is the extensive sequence polymorphism of plasmodial antigens, which facilitates the immune evasion of the different isolates (lacking cross-coverage of those isolates resulting in missing cross-protection).

Research towards the development of malaria vaccines has been pursued since the 1960s, but Plasmodium falciparum the causative agent of malaria tropica still poses daunting challenges to scientists, as there is still no licensed malaria vaccine available yet. Major obstacles that have impeded developing antimalarial vaccines include the diverse antigenic repertoire associated with the parasites many life-cycle stages, antigenic variation and diversity of wild-type isolates as well as restricted host genetic responsiveness.

Immunity against malaria parasites is stage dependent and species dependent. Many malaria researchers and textbook descriptions believe and conclude that a single-antigen vaccine representing only one stage of the life cycle will not be sufficient and that a multiantigen, multistage vaccine that targets different, that is at least two, stages of parasite development is necessary to induce effective immunity (Mahajan, Berzofsky et al. 2010). The construction of a multiantigen vaccine (with the aim of covering different parasite stages and increasing the breadth of the vaccine-induced immune responses to try to circumvent potential Plasmodium falciparum escape mutants) can be achieved by either genetically linking (full-size) antigens together, by a mixture of recombinant proteins or by synthetic-peptide-based (15-25-mer), chemically synthesized vaccines containing several peptides derived from different parasite proteins and stages.

A single fusion protein approach being comprised of several different antigens or several different alleles of a single antigen (to induce antibodies with synergistic activities against the parasite) is hindered by antigenic diversity and the capacity of P. falciparum for immune evasion (Richards and Beeson, 2009). A large number of antigens have been evaluated as potential vaccine candidates, but most clinical trials have not shown significant impact on preventing clinical malaria although some of them have shown to reduce parasite growth. The size of the resulting fusion protein/vaccine candidate is another limiting factor allowing only the combination of a few selected antigens, not excluding that the chosen antigens are not targets of natural immunity and/or exhibit significant genetic polymorphism. Highly variable antigens with multiple alleles are obviously targets of the immune response under natural challenge, and vaccine studies of PfAMA1 and PfMSP2 suggest that allele-specific effects can be achieved (Schwartz, 2012). Currently only combination vaccines (being comprised of PfCSP and PfAMA1) are undergoing clinical trials that target the pre-erythrocytic and asexual blood stage of P. falciparum (Schwartz, 2012). A multiantigen vaccine candidate, neither a fusion, nor a combination approach, targeting all three life cycle main stages of Plasmodium (including the sexual stage in Anopheles mosquitos and thus blocking parasite transmission) has still not been tested in clinical trials.

Therefore the availability of novel and improved multicomponent, in particular multi-stage human and/or animal vaccines potentially would be highly advantageous.

### SUMMARY OF THE DISCLOSURE

The present disclosure relates to novel protein complexes suitable as vaccines comprising recombinant fusion protein units. The present disclosure pertains in particular to isolated recombinant ImmunoAssemblins (IAs), in particular to antibody-like recombinant fusion protein complexes suitable as animal or human vaccines against infectious diseases caused by virus, bacteria and/or eukaryotic parasites, auto-immune diseases and cancer, wherein the IAs comprise a plurality of antigens or antigen domains derived from proteins preferably, but not necessarily presented on the surface of a pathogen or cell. In particular, the novel type complexes of recombinant polypeptides comprise a plurality of different antigens from a single or more than one pathogen, and/or a plurality of variants of the same antigen, and/or at least one component that is not derived from a pathogen.

In a first aspect, the present disclosure pertains to immunoassemblin (IA) protein complex suitable as a vaccine comprising at least three recombinant fusion protein units, wherein:
a) the first fusion protein unit comprises the immunoglobulin heavy chain constant domains C_{H}1 and C_{H}3 and a first antigen, wherein said first antigen is linked N-terminal and/or C-terminal to at least one of the immunoglobulin heavy chain constant domains (HC fusion polypeptide unit 1, HC unit 1); and
b) the second fusion protein unit comprises an immunoglobulin light chain constant domain C_{L}, and a second antigen, wherein said second antigen is linked N-terminal and/or C-terminal to the C_{L}-domain (LC fusion polypeptide unit 1, LC unit 1), and
c) the third fusion protein unit comprises the immunoglobulin heavy chain constant domains C_{H}1 and C_{H}3 and a third antigen, wherein said third antigen is fused N-terminal and/or C-terminal to the immunoglobulin heavy chain constant domains of said third fusion protein, or
d) the third fusion protein unit comprises an immunoglobulin light chain constant domain C_{L}, and a third antigen, wherein said third antigen is fused N- or C-terminal to the C_{L}-domain, and wherein
   said antigens of said three recombinant fusion protein units differ in their amino acid sequence.
   Some aspects of the present disclosure relates to IA protein complexes, wherein
a) a first fusion protein unit comprises the immunoglobulin heavy chain constant domains C_{H}1 and C_{H}3 and a first antigen, wherein said first antigen is fused N-terminal to the C_{H}1-domain (HC unit 1); and
b) said second fusion protein unit comprises an immunoglobulin light chain constant domain C_{L}, and a second antigen, wherein said second antigen is fused N-terminal to the C_{L}-domain (LC unit 1), and wherein said first and said second fusion protein unit are covalently linked to each other, in particular by at least one disulfide bond,
c) said third fusion protein unit comprises the immunoglobulin heavy chain constant domains C_{H}1 and C_{H}3 and a third antigen, wherein said third antigen is fused N-terminal to the C_{H}1-domain of said third fusion protein unit (HC unit 2), wherein said HC unit 1 and HC unit 2 are covalently linked to each other, in particular by at least one disulphide bond,
d) said fourth fusion protein unit comprises an immunoglobulin light chain constant domain C_{L}, and a fourth antigen, wherein said fourth antigen is fused N-terminal to the C_{L}-domain of said fourth fusion protein (LC unit 2), and wherein said third and the fourth fusion protein unit are covalently linked to each other, in particular by at least one disulfide bond.

Nucleic acid molecules encoding said recombinant fusion proteins comprised in the IA protein complexes, vectors and host cells containing said nucleic acids and methods for preparation and producing such fusion proteins and/or protein complexes are also disclosed, as well as antibodies induced or generated by the use of said complexes suitable a vaccines and the use of such complexes and/or antibodies or recombinant derivatives thereof for immunotherapy.

Therefore, in a further aspect, embodiments of this disclosure relate also to vaccine compositions comprising a protein complex according to the present disclosure and a pharmaceutically acceptable carrier and/or adjuvant. In some advantageous embodiments, this disclosure relate also to vaccine compositions comprising a protein complex according to the present disclosure without an additional adjuvant.

In another aspect, the present disclosure is directed to antibody compositions comprising different isolated antibodies or fragments thereof binding to an IA protein complex according to the present disclosure or antibody compositions comprising different isolated antibodies or fragments thereof binding to the different recombinant proteins in the IA protein complex according to the present disclosure.

In a further aspect, embodiments of this disclosure relate to vaccine compositions for immunizing a mammal, in particular a human, in particular against malaria comprising as an active ingredient comprising a protein complex according to the present disclosure and a carrier in a physiologically acceptable medium.

Furthermore, methods of immunizing humans against a pathogen, in particular against a Plasmodium infection, in particular against Plasmodium falciparum, comprising administering an effective amount of an IA protein complex of the present disclosure, a composition comprising an IA protein complex of recombinant fusion proteins of the present disclosure or a vaccine composition according to the present disclosure are disclosed.

Another aspect pertains to methods of producing an IA protein complex according to the present disclosure comprising the steps of:
a) culturing a host cell according to the present disclosure in a suitable culture medium under suitable conditions to produce said recombinant fusion proteins, wherein the host cell comprises all nucleic acid molecules encoding the fusion proteins units of an isolated IA protein complex according to the present disclosure, and wherein said IA protein complex is formed in the host cell,
b) isolating said IA protein complex, and optionally
c) processing said IA protein complex.

Another aspect pertains to methods of producing an IA protein complex according to the present disclosure comprising the steps of:
(a) culturing of a plurality of host cells according to the present disclosure in a suitable culture medium under suitable conditions to produce said recombinant fusion proteins;
(b) isolating said produced fusion proteins,
(c) mixing said fusion proteins to produce said IA protein complex
(d) isolating said produced IA protein complex, and optionally
(e) processing the IA protein complex.

Another important aspect pertains to isolated IA protein complex suitable as a vaccine comprising (i) at least two recombinant fusion proteins, (ii) at least two different antigens, (iii) at least one different homo- and/or hetero-oligomerization domain, wherein said first recombinant fusion protein comprise at least one homo- or hetero-oligomerization domain that is absent from said second recombinant fusion protein.

Another important aspect pertains to vaccine compositions comprising at least two different immunoassemblin protein complexes according to the present disclosure, i.e a mixture of different IA protein complexes comprising a variety of different antigens or antigen compositions.

Before the disclosure is described in detail, it is to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. It is moreover to be understood that, in case parameter ranges are given which are delimited by numeric values, the ranges are deemed to include these limitation values.

### BRIEF DESCRIPTION OF THE DRAWINGS

FIG. 1 is a schematic depiction of a state-of-the-art immunoadhesin. The protein of interest replaces variable antibody regions (VH+VL, Fv) followed by the hinge, C_{H}2 and C_{H}3 domains of an IgG1 heavy chain. Gained beneficial features are enumerated on the right side.

FIG. 2 is a scheme of the life cycle of Plasmodium falciparum. The schematic depiction illustrates the complex and multi-stage life cycle of the most lethal malaria causing agent P. falciparum. The three major/main phases of the Plasmodium life cycle are: the pre-erythrocytic stage taking place in the liver, the asexual blood-stage occurring inside infected red blood cells (RBCs) and the sexual stage is carried out within the mosquito

FIG. 3 is a scheme showing the structure of a human IgG1 antibody (left) compared to an embodiment of an IA protein complex, e.g. a malaria immunoassemblin (MIA, right). The chosen protein of interest can be any of a variety of functional types, including (malaria) antigens/adhesins, receptor extracellular domains, cytokines, interleukins, enzymes, toxins etc. Compared to the immunoglobulin Fc-region (hinge, C_{H}2, C_{H}3) of a classical state-of-the-art immunoadhesin, a MIA molecule may further comprises C_{H}1 domains as well as constant kappa light chains N-terminally fused to malaria adhesins. The Fc-backbone imparts in vivo stability via FcRn binding, among other potential effector functions shown.

FIG. 4 shows a Coomassie-staining and immunoblot analysis of the first two MIA combinations MSP1₁₉_{_}3D7-HC:Pfs25_{FKO}-LC and MSP1₁₉_{_}3D7-HC:Pfs28-LC. A+C: Coomassie-stained polyacrylamid gels (12%) of reduced purification samples; B+D: Western blots of pooled and dialyzed elution fractions E1 and E2 of both purifications under reducing and non-reducing conditions. Aliquots of 10 μL for each sample were loaded. Immunodetection of MIA heavy and light chain fusion proteins was performed using alkaline phosphatase probed antibodies GAH-Fc-AP and GAH-LCkappa-AP simultaneously. Visualization of target proteins bands was achieved after addition of NBT/BCIP substrate and short incubation in the dark (2-3 min). M: PageRuler™; bpH: sample before pH shift; L: processed plant extract (load); F: flow-through of plant extract; W: wash sample of used Protein A column (10 CV, 1×PBS+500 mM PBS, pH 7.4); E1-E5: analyzed elution fractions.

FIG. 5 is a scheme showing the structure of a human IgG1 based malaria immunoassemblin (MIA, left) as a building block for derived malaria immunoassemblins with additional C-terminal fused candidate antigens (MIA-Cs, right)

FIG. 6 shows purification samples of dual-stage covering Tetra_MSP1₁₉-HC-CSP_{TSR}-ERH and its protein-analytical assessment. A) Coomassie-stained polyacrylamid gel (12%) of reduced purification samples; B) Stained SDS-gel and Western blot of pooled and dialyzed elution fractions E1 and E2. Aliquots of 10 μL for each sample were loaded. Immunodetection of MIA-C fusion protein was performed using alkaline phosphatase probed antibody GAH-Fc-AP. Visualization of target protein bands was achieved after addition of NBT/BCIP substrate and short incubation in the dark (2-3 min). M: PageRuler™; L: processed plant extract (load); F: flow-through of plant extract; W: wash sample of used Protein A column (10 CV, 1×PBS+500 mM PBS, pH 7.4); E1-E5: analyzed elution fractions.

FIG. 7 shows a Coomassie stained polyacrylamide gel of the migration pattern differences between unmodified HCs variant MSP1₁₉-HC:AMA1_{GKO}-HC and mutated variant MSP1₁₉-modHC1_E356K-D399K:AMA1_{GKO}-modHC2_K392D-K409D under reducing and non-reducing conditions. A) Insertion of charge pair mutations enables creation of heterodimeric MIA-HCs B) Coomassie-stained polyacrylamid gel (12%) of said unmodified and mutated MIA-HC variants under reducing conditions. C) Very same samples under non-reducing conditions. All samples were incubated at 70° C. for 10 min prior to SDS-PAGE. Cartoons right to the stained gel image extracts were integrated in order to illustrate and facilitate comprehension. *: LC polypeptide unit(s) were excluded in this experiment to simplify the visual illustration of assembly differences. In later experiments, LC units were co-expressed together with two complementary chair pair mutations including HC units.

FIG. 8 shows an overview of the different IA protein complex constructs, e.g. MIA protein complexes and their minimal versions lacking C_{H}2 domains.

FIG. 9 shows the inherent combinatorial potential of the immunoassemblin (IA) approach according to the present disclosure. The simplest IA format was chosen to visualize the combinatorial potential using the herein described HC and LC fusion polypeptides (HC and LC units). For simpler presentation purposes the number (n) of HCs and LCs included was limited to n=2. Increasing the quantity of involved components (n>2) highly increases the resulting extent of combinations thereof.

FIG. 10 shows a cartoon (A) of the assembled MIA vaccine candidate ARC25 presenting its molecular composition comprising malaria vaccine antigens and their stage-of-action.

FIG. 11 I illustrates the coomassie stained gels post ARC25 protein A chromatography with subsequent preparative gel filtration under reducing conditions. Expected target protein bands are marked with an arrow and protein ID on the left. M: Protein ladder Page Ruler; L: load; F: flow-through; W: wash fraction; E1-E6: protein A elution samples 1 to 6; A7-A11: gel filtration samples including target protein.

FIG. 12 shows an immunolabelling of the pre-erythrocytic and the asexual blood stage of P. falciparum with rabbit immune sera generated against ARC25 and BSSC. IFAs were performed on methanol-fixed sporozoites and blood-stage schizonts using the protein A-purified rabbit IgGs from rabbit serum samples collected on day 70 after immunization with either ARC25 or BSSC. Exemplary shown are two rabbit IgG samples (rabbit no. 24701 for ARC25) rabbit no. 24704) immunolabeled the whole surface of sporozoites as well as the apical pole of merozoites enclosed by the schizonts. Mouse-anti-PfCSP_TSR monoclonal antibody 6.75M was applied to detect the sporozoite surface and mouse anti-PfMSP1₁₉ antiserum was used to counterstain the merozoite plasmalemma; the parasite nuclei were highlighted with Hoechst 33342. Purified IgG-fractions of immunized rabbits were used at a concentration of 15 mg/mL and were evaluated for binding to the native P. falciparum surface.

FIG. 13 shows two examples of novel IA protein complexes. 12A) ARC25 Admixture (ARC25Ad) consists of experimental MIA vaccine candidate ARC25 and a synthetic RON2L peptide. Binding of RON2L into the hydrophobic pocket of AMA1 results in unraveling of epitopes for superior inhibitory antibodies. 12B) A monovalent version of anti-AMA1 inhibitory mAb 1E4 with C-terminal malaria antigen fusion proteins in complex with any AMA1 variant.

FIG. 14 shows a further example of a novel IA protein complex. OptARC25 was produced the same way as its first generation equivalent. C-terminal addition of RON2L to the Rh2_GKO-modHC2.2 resulted in an immune string-like complex of enormous molecular weight exceeding 2 MegaDa.

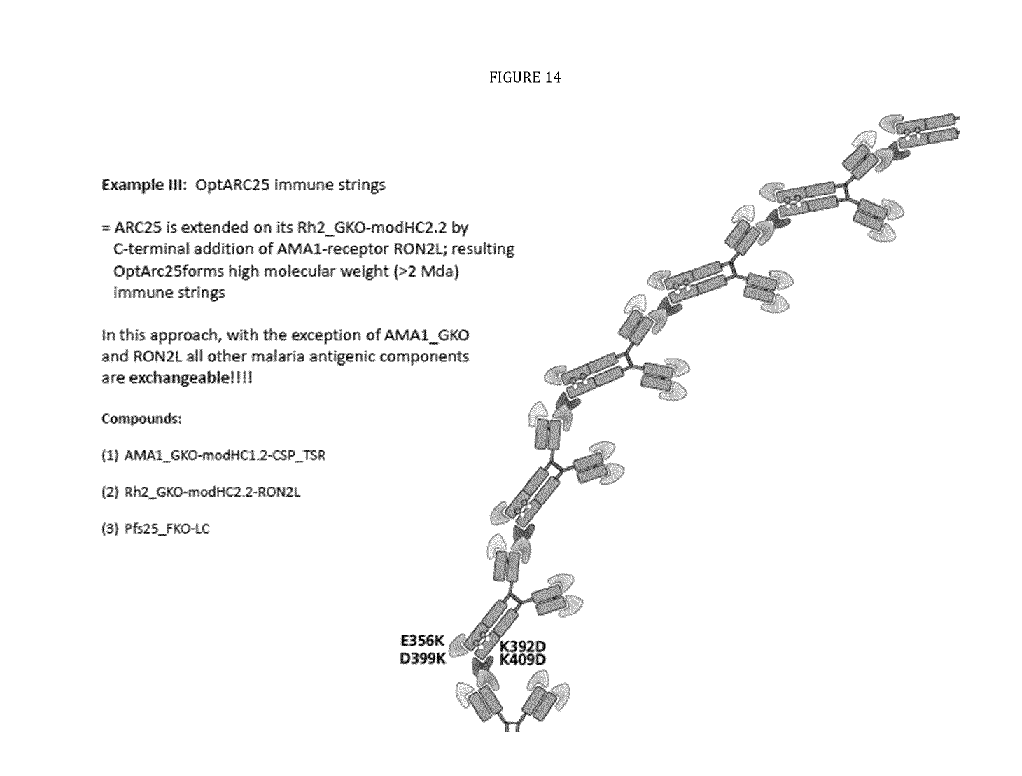

FIG. 15 shows a further example of a novel IA protein complex. A heterotrimeric IA protein complex potentially including at least three antigenic components as well as minimally three proteineacous elements of different functionality (receptor portions, cytokines, toll-like recoptor ligands, interleukine etc.)

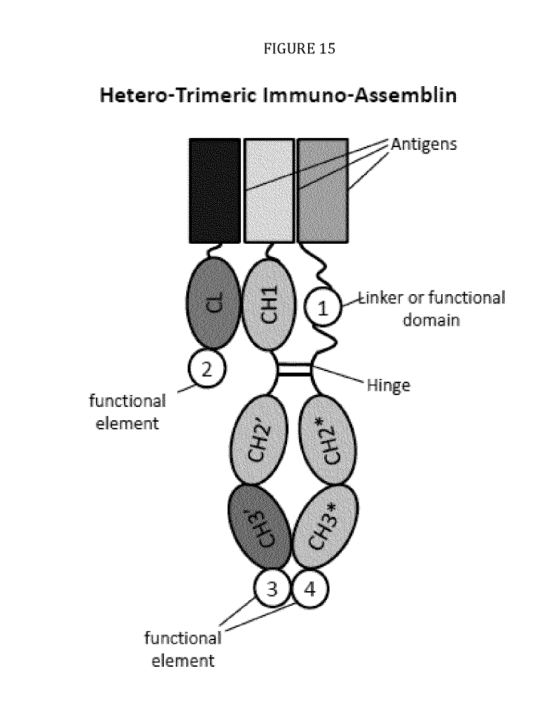

FIG. 16 shows a coomassie-stained poly-acrylamid gel of samples from the purification of “HexaMix” (left side) and immunoblot analysis of the individual components using the pooled and dialyzed E1-E6 elution fractions (right side).

FIG. 17 shows a coomassie-stained PAA gel of multi-Disease IA (mDIA) purification samples and immunoblots of dialyzed elution samples E1.

### DETAILED DESCRIPTION OF THIS DISCLOSURE

As mentioned above, the present disclosure relates to novel protein complexes suitable as vaccines comprising recombinant fusion protein units. The present disclosure pertains in particular to isolated recombinant ImmunoAssemblins (IAs), in particular to antibody-like recombinant fusion protein complexes suitable as animal or human vaccines against infectious diseases caused by virus, bacteria and eukaryotic parasites, auto-immune diseases and cancer, wherein the IAs comprise a plurality of antigens or antigen domains derived from proteins preferably, but not necessarily presented on the surface of a pathogen or cell. In particular, the novel type complexes of recombinant polypeptides comprise a plurality of different antigens from a single or more than one pathogen, and/or a plurality of variants of the same antigen, and/or at least one component that is not derived from a pathogen.

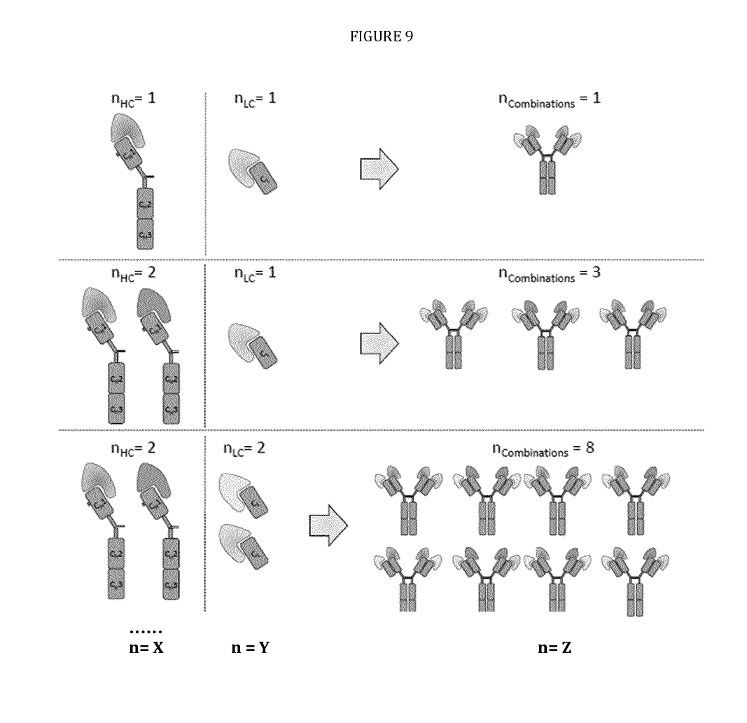

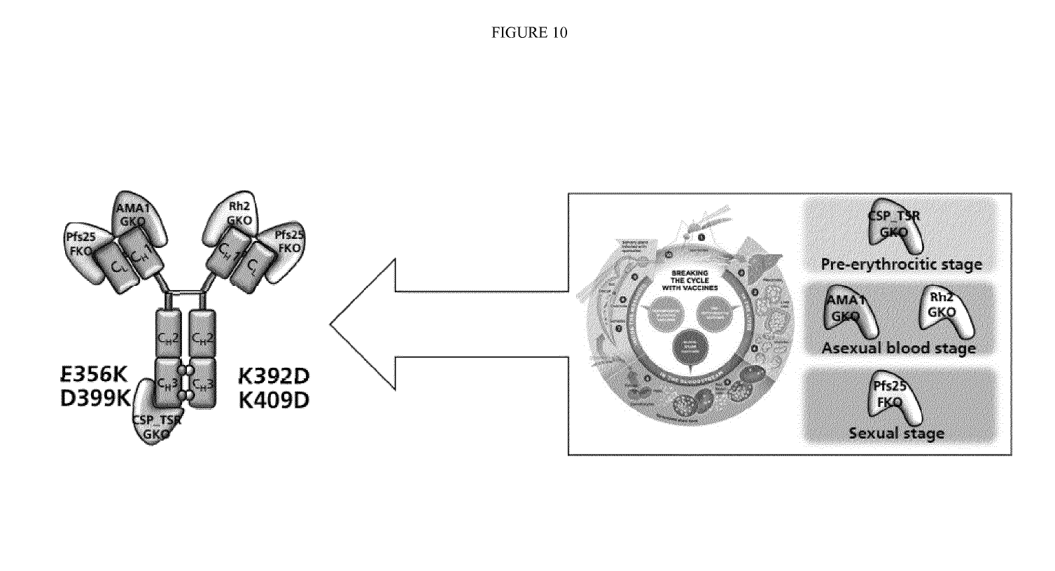

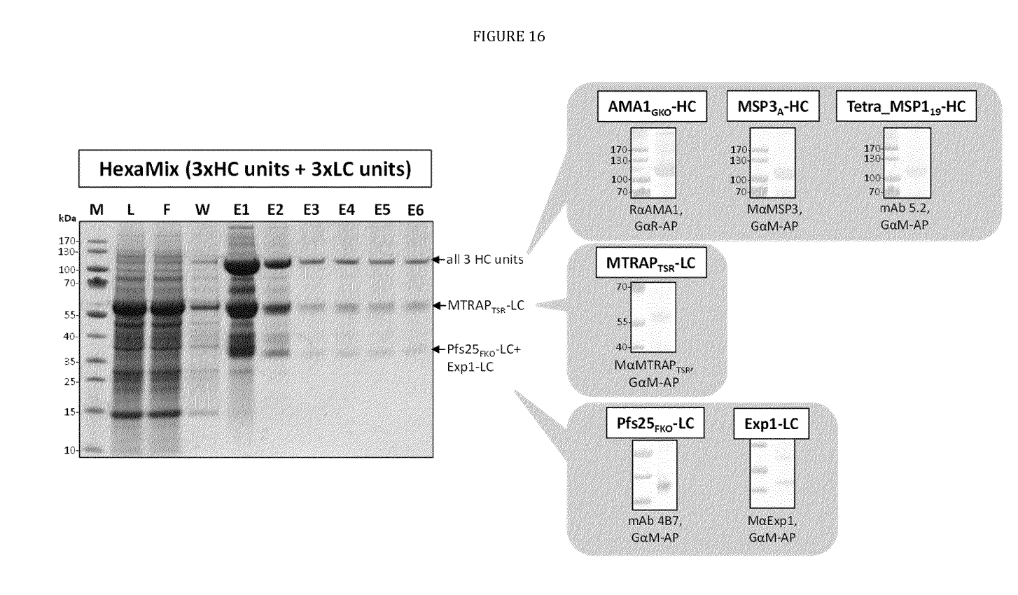

In particular, embodiments of the present disclosure pertains to Immunoassemblins (IAs) including C_{H}1 domains and light chains in their composition other than classical immunoadhesins that are traditionally produced as Fc-homodimers lacking C_{H}1 domains and light chains. IAs of the present disclosure enable the possibility of assembling different heavy and light chain fusion proteins in one single molecule (see FIG. 10), or combining a variety of different fusion polypeptides in a mixture or cocktail (see FIG. 9, FIG. 16). Thereby, not only an immunological-relevant core is provided as a scaffold for the exposure of multiple important vaccine candidates, therapeutics or other drugs, furthermore a solution for the mentioned laborious efforts of multi-component vaccines is elegantly addressed by modern protein design.

A further advantage is that these mixtures and vaccine cocktails of the novel type complexes of recombinant polypeptides may be generated by co-expression of several encoding genes within the same expression host, tissue and/or cell (see FIG. 16) or by expressing several genes individually and subsequently combining them. These mixtures and derived vaccine formulations (cocktails) comprise multiple HC fusion polypeptides units and multiple LC fusion polypeptides units, which may be purified using a single generic chromatography method, i.e. protein-A chromatography (see FIG. 16). This demonstrates that the IA according to this invention enable the rapid inclusion of new antigens, thereby facilitating rapid responses to seasonal strain fluctuations, or other emerging diseases.

Furthermore, the IA protein complexes according to the present disclosure lead to improvements of immunological host responsiveness by including human IgG1 constant regions to increase the effector functions/interactions with immune cells of generated combinatorial molecules. Furthermore, the production methods (production system) for the IA protein complexes are easily up-scalable and cost-effective production system and for example enables the utilization of a single generic chromatography method (protein-A chromatography) for all possible protein combinations with regards to the terms of regulatory authorities as well as good manufacturing practice.

In particular, the novel type protein complexes comprise a plurality of different antigens from a single or more than one pathogen, and/or a plurality of variants of the same antigen, and/or at least one component that is not derived from a pathogen. Furthermore, the IA protein complexes according to the present disclosure may comprise several functional units, i.e. at least one antigen, at least two different protein domains mediating homo or heteromeric assembly, and at least one protein domain capable of interacting with receptors on cells of the immune system. Embodiments of the present disclosure pertains to IA protein complexes suitable as vaccines for malaria tropica, recombinant proteins composed of malaria antigens and fusion proteins thereof, that are genetically fused to human IgG1 constant heavy and light chain domains. By co-expressing different malaria antigen including recombinant HC and LC fusion proteins, efficacious multicomponent and multistage malaria vaccine candidates covering all main Plasmodium falciparum life-cycle stages (the pre-erythrocytic, the asexual blood- and the sexual-stage) are generated and used to elicit protective immune responses in humans.

In the present disclosure, novel solutions for several of the above-mentioned vaccine limitations are shown. For example, the antibody constant domains or engineered variants thereof, excert immunomodulatory effects through interaction with Fc-receptors on immune-cells. In addition, other immunomodulatory properties can be introduced by fusion or linkage with e.g. cytokines or interleukins, Toll-like receptor ligands such that the molecules according to the present disclosure themselves exhibit and provide for the adjuvant properties.

In this context, it was surprisingly observed, that the IA protein complexes according to the present disclosure are particularly suited for inducing responses against all represented antigens. Furthermore, the IA protein complexes according to the present disclosure are easy to produce and therefore offer cost-effective vaccine approaches. The IA protein complexes according to the present disclosure may not only comprising multiple antigens but also multiple functionalities, represented by the antigens, the antibody constant domains and other effector molecules, such as cytokines, interleukins, toll-like receptor ligands, that are fused or linked to the polypeptides of the immunoadhesins, and which function as adjuvant.

The Immunoadhesins of the present disclosure are particularly suited for generating unique assemblies derived from homo- and hetero-oligomeric proteins found on the surface of many pathogens, especially viruses. Target molecules for example include but are not limited to the envelope from HIV, hemagglutinin from Influenza virus, rabies virus glycoprotein, E-glycoprotein from Flaviviruses such as Dengue Virus, West Nile Virus, Yellow Fever Virus, tick borne encephalitis Virus, Hepatitis Virus C envelope glycoproteins E1 and E2, Ebola Virus GP1,2, BMFP, a basic trimeric coiled-coil protein with membrane fusogenic activity from Brucella abortus, YqiC of Salmonella enterica serovar Typhimurium; According to the present disclosure, these assemblies can comprise one or more antigens from the same or from different pathogens, comprise multiple sequence variants of the same antigen and further comprise one or more other functionalities, e.g. with adjuvant properties. Examples of further antigens are the polypeptides comprising any one of the amino acid sequences of SEQ ID NO. 105 to SEQ ID. NO. 107.

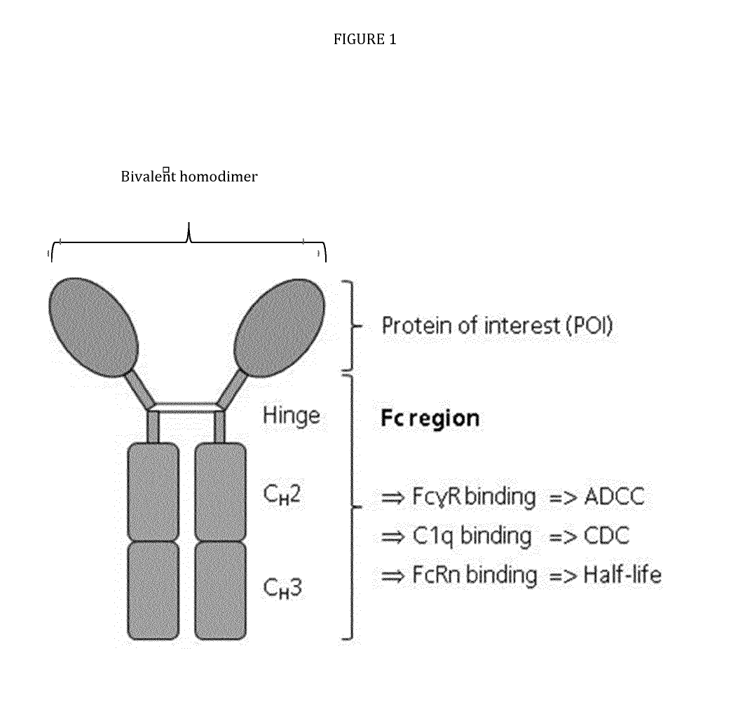

Immunoadhesins are chimeric proteins, in particular antibody-like molecules that are amino (N)-terminally composed of any proteinaceous molecule of interest (antigen) joined for example to a carboxy (C)-terminus containing the hinge, C_{H}2 and C_{H}3 regions of a human IgG1 heavy chain (see FIG. 1). The non-Ig part of the chimeric molecule may be functionally analogous to the variable regions of an IgG, whose role is to mediate target recognition. Inclusion of the Fc-part confers beneficial biological, immunological as well as pharmacological properties of human antibodies, like placental transfer, complement fixation and prolonged serum half-life due to target protein recycling by binding to the salvage neonatal Fc-receptor (FcRn), thus preventing lysosomal degradation (Mekhaiel et al., 2011). Furthermore, effector functions are mediated as a result of interactions with Fc-receptors that are present on certain cells of the immune system, e.g. natural killer (NK) cells and antigen presenting cells (APCs) (Perez de la Lastra et al., 2009). Moreover, from a technological viewpoint the Fc-region allows the utilization of an easy and cost-effective, but more important a generic purification method by protein-A/G affinity chromatography. Since their first description in 1989, when a number of CD4-immunoglobulin hybrid molecules were intended and produced as potential AIDS therapeutics (Capon et al., 1989), almost all immunoadhesins were expressed as Fc-homodimers lacking C_{H}1 domains and light chains due to their envisaged/desired/targeted single “functionality”, as those IgG elements were found to be expandable and undesirable.

An advantages is that mixtures of the novel type complexes of recombinant polypeptides may be generated by co-expression of several genes encoding within the same host cell or by expressing several genes individually and subsequently combine them. The novel type complexes of recombinant polypeptides or their components may be purified individually or as mixture.

Furthermore, the IA protein complexes according to the present disclosure leads to improvements of immunological host responsiveness by including human IgG1 constant regions to increase the effector functions/interactions with immune cells of generated combinatorial molecules. Furthermore, the production methods (production system) for the IA protein complexes are easily up-scalable and cost-effective production system and for example enables the utilization of a single generic chromatography method for all possible protein combinations with regards to the terms of regulatory authorities as well as good manufacturing practice.

The IA protein complexes according to the present disclosure are suitable as vaccines for pathogens with complex life cycles and with multiple stages and plenty of potential target antigens. Therefore, the use of a plurality of different antigens, in particular of antigens representing more than one life cycles lead to a robust immunity.

The desire for a vaccine candidate composed of a single polypeptide is mainly driven by practical, technical and economical demands for reproducible, robust and cost-efficient production. However, to those skilled in the art, it is also clear, that there is a size limitation for recombinant expressed proteins. Although protein specific differences have to be taken into account as well, there is a strong decrease of expression levels and yields with increasing length of the polypeptide. Multiple challenges increase over-proportionally with size and the overall properties of large proteins are significantly less amenable to optimization than those of smaller proteins, domains or fragments. All these problems have so far been significant bottlenecks for the development of efficient vaccines against apicomplexan parasites and have resulted in an overwhelming number of sub-optimal vaccine candidates that comprise only multiple linear epitopes, one or two antigens from a one or two life cycle stages. As alternative, chemically or genetically attenuated or inactivated life-vaccines are proposed (e.g. irradiated sporozoites), but such approaches have to deal with batch-to-batch consistency, scaled-up production and most importantly product safety.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

An immunoassemblin (IA) complex according to the present disclosure is a hetero-oligomeric protein complex comprising resulting from the expression of at least three different recombinant genes. The different recombinant genes may or may not be expressed within the same expression host. In an advantageous embodiment the three or more different recombinant genes are expressed in the same expression host.

In another embodiment of the present disclosure, the three or more different recombinant genes are co-expressed within the same cell. However, those skilled in the art will appreciate that there are technical and biological limits and that this does not imply that all three or more different recombinant genes are expressed in each and every single cell, i.e. 100% of the host cells, and that according to the present disclosure “co-expressed within the same cell” means that the three or more different recombinant genes are expressed in the majority, i.e. at least 50%, preferably at least 70% and more preferably at least 85% of the host cells.

Those skilled in the art will know that the genetic information for expressing the three or more different recombinant genes can be encoded by various nucleic acids, including but not limited to plasmid DNA, double stranded DNA, DNA fragments, single stranded DNA, double stranded RNA and single stranded RNA. The nucleic acids can be in both sense and antisense, single or double stranded, circular or linear and can be free, i.e. naked, or covered by proteins, polymers, liposomes and the like. Moreover, the genetic information may be carried by a virus, bacteria or other organism. Those skilled in the art also understand that there are a variety of methods for delivering genetic information to cells, tissues and organisms (bacteria, microbes, plant and mammalian cells). Furthermore, the three or more different recombinant genes or the genetic information encoding the three or more fusion protein units, may be physically linked and e.g. be encoded on the same plasmid, but may also be separated on distinct nucleic acids or a combination of linked and unlinked arrangements may be used.

Co-expression of three or more different recombinant genes, and assembly of the resulting individual polypeptide chains (fusion protein units) may lead to a discrete protein complex, as e.g. demonstrated in case of ARC25 (see examples), but may also lead to a mixture of protein complexes comprising variable amounts of the fusion protein units. The formation of a single immunoassemblin (IA) complex, or two or more immunoassemblin (IA) complexes, may occur during expression and/or incubation and/or extraction and/or further downstream processing and/or formulation. One or more immunoassemblin (IA) complexes may be produced using a single upstream process or may be produced using two or more separate upstream processes. Likewise, one or more immunoassemblin (IA) complexes may be present in and processed by the same downstream processing step, e.g. including but not limited to heating, extraction, filtration, chromatography or viral inactivation. In an advantageous embodiment the immunoassemblin (IA) complex or complexes are obtained from the same purification process.

The present disclosure pertains to isolated immunoassemblin (IA) protein complexes suitable as a vaccine comprising at least two recombinant fusion protein units, wherein:
a) the first fusion protein unit comprises the immunoglobulin heavy chain constant domains C_{H}1 and C_{H}3 and a first antigen, wherein said first antigen is linked N-terminal and/or C-terminal to at least one of the immunoglobulin heavy chain constant domains (HC fusion polypeptide unit 1, HC unit 1); and
b) a second fusion protein unit comprising an immunoglobulin light chain constant domain C_{L}, and a second antigen, wherein said second antigen is linked N-terminal and/or C-terminal to the C_{L}-domain (LC fusion polypeptide unit 1, LC unit 1), and wherein
c) said first and said second antigen comprises different amino acid sequences.

In particular, present disclosure pertains to isolated immunoassemblin (IA) protein complexes suitable as vaccines comprising at least three recombinant fusion protein units, wherein:
a) the first fusion protein unit comprises the immunoglobulin heavy chain constant domains C_{H}1 and C_{H}3 and a first antigen, wherein said first antigen is linked N-terminal and/or C-terminal to at least one of the immunoglobulin heavy chain constant domains (HC fusion polypeptide unit 1, HC unit 1); and
b) the second fusion protein unit comprises an immunoglobulin light chain constant domain C_{L}, and a second antigen, wherein said second antigen is linked N-terminal and/or C-terminal to the C_{L}-domain (LC fusion polypeptide unit 1, LC unit 1), and
c) the third fusion protein unit comprises the immunoglobulin heavy chain constant domains C_{H}1 and C_{H}3 and a third antigen, wherein said third antigen is fused N-terminal and/or C-terminal to the immunoglobulin heavy chain constant domains of said third fusion protein, or
d) the third fusion protein unit comprises an immunoglobulin light chain constant domain C_{L}, and a third antigen, wherein said third antigen is fused N- or C-terminal to the C_{L}-domain, and wherein
e) said antigens of said three recombinant fusion protein units differ in their amino acid sequence.

In further advantageous embodiments, the isolated protein complex according to the present disclosure comprises as the third recombinant fusion protein unit the immunoglobulin heavy chain constant domains C_{H}1 and C_{H}3 and a third antigen, wherein said third antigen is fused N-terminal and/or C-terminal to the immunoglobulin heavy chain constant domains of said third fusion protein (HC unit 2).

In further advantageous embodiments, the isolated protein complex according to the present disclosure comprises a fourth recombinant fusion protein comprising an immunoglobulin light chain constant domain C_{L}, and a further antigen, wherein said further antigen is fused N- or C-terminal to the C_{L}-domain (LC unit 2), and wherein the two LC units comprise preferably different amino acid sequences.

According to the present disclosure, the first fusion protein unit (HC unit 1) and the second fusion protein unit (LC unit 1) may be linked to each other. Further, the first fusion protein unit (HC unit 1) and said third fusion protein unit (HC unit 2) may be linked to each other. Furthermore, the third fusion protein unit (HC unit 2) and the fourth fusion protein unit (LC unit 2) may be linked to each other.

In some further embodiments or the present disclosure, the fusion protein units may be covalently or non-covalently linked to each other in the protein complex of the present disclosure. According to the present disclosure the term “covalently linked” comprises a covalent bond that involves the sharing of electron pairs between atoms. These electron pairs are known as shared pairs or bonding pairs and the stable balance of attractive and repulsive forces between atoms when they share electrons is known as covalent bonding. The term “non-covalently linked” comprises a non-covalent interaction that differs from a covalent bond in that it does not involve the sharing of electrons, but rather involves more dispersed variations of electromagnetic interactions between molecules or within a molecule. In some advantageous embodiments of the present disclosure, the fusion proteins are covalently linked to each other by a disulphide bond.

Therefore, in the present disclosure the term “Linked” includes non-covalent or covalent bonding between two or more molecules. Linking may be direct or indirect. Two molecules are indirectly linked when the two molecules are linked via a connecting molecule (linker), like a hinge region. Two molecules are directly linked when there is no intervening molecule linking them. The fusion protein units comprised in the IA protein complexes according to the present disclosure may be covalently or non-covalently linked to each other. In some advantageous embodiments, the fusion protein units comprised in the IA protein complexes according to the present disclosure may be covalently linked to each other by a disulfide bond.

A “protein complex” according to the present disclosure is a composition comprising two or more polypeptides. In advantageous embodiments of the present disclosure the term “protein complex” is directed to a group of two or more associated polypeptide chains. The different polypeptide chains may have different functions. Protein complexes may be a form of quaternary structure. Proteins in a protein complex may be linked by covalent and/or non-covalent protein-protein interactions, and different protein complexes may have different degrees of stability over time. In an advantageous embodiment of the present disclosure, the protein complex is an antibody-like protein complex.

A typical antibody is an immunoglobulin molecule comprised of four polypeptide chains, two heavy (H) chains (about 50-70 kDa when full length) and two light (L) chains (about 25 kDa when full length) inter-connected by disulfide bonds. Light chains are classified as kappa and lambda. Heavy chains are classified as gamma, mu, alpha, delta, or epsilon, and define the antibody's isotype as IgG, IgM, IgA, IgD, and IgE, respectively.

Each heavy chain is comprised of a heavy chain variable region (abbreviated herein as VH) and a heavy chain constant region. The heavy chain constant region is comprised of three domains (C_{H}1, C_{H}2, and C_{H}3) for IgG, IgD, and IgA; and 4 domains (C_{H}1, C_{H}2, C_{H}3, and C_{H}4) for IgM and IgE. Each light chain is comprised of a light chain variable region (abbreviated herein as VL) and a light chain constant region. The light chain constant region is comprised of one domain, C_{L}. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The assignment of amino acids to each domain is in accordance with well-known conventions (Chothia and Lesk 1987; Chothia et al. n.d.; Kabat et al. 1992). The functional ability of the antibody to bind a particular antigen is largely determined by the CDRs.

In some advantageous embodiments, the immunoassemblin (IA) protein complexes of the present disclosure are antibody-like protein complex comprising at least two heavy chain constant regions C_{H}1, and C_{H}3, in particular three heavy chain constant regions C_{H}1, C_{H}2 and C_{H}3, and an immunoglobulin light chain constant domain C_{L}, wherein an antigen is linked/fused to heavy chain constant region and another antigen is linked/fused to the immunoglobulin light chain constant domain.

However, in advantageous embodiments, the immunoassemblin (IA) protein complex suitable as a vaccine according to the present disclosure are antibody-like protein complexes but does not comprise an antibody VH and/or VL region.

In some advantageous embodiments, the IA protein complex is an isolated protein complex. The term “isolated” when used in relation to a protein complex, a protein or a nucleic acid refers to a nucleic acid sequence, protein or protein complex that is identified and separated from at least one contaminant (nucleic acid or protein, respectively) with which it is ordinarily associated in its natural source. Isolated nucleic acid or protein is present in a form or setting that is different from that in which it is found in nature. In contrast, non-isolated nucleic acids or proteins are found in the state they exist in nature.

The terms “polypeptide”, “peptide”, or “protein” are used interchangeably herein to designate a linear series of amino acid residues connected one to the other by peptide bonds between the alpha-amino and carboxyl groups of adjacent residues. The amino acid residues are preferably in the natural “L” isomeric form. However, residues in the “D” isomeric form can be substituted for any L-amino acid residue, as long as the desired functional property is retained by the polypeptide. In addition, the amino acids, in addition to the 20 “standard” amino acids, include modified and unusual amino acids.

In some advantageous embodiments, an isolated IA protein complex according to the present disclosure is suitable as a vaccine, in particular as a human and/or animal vaccine. A “vaccine” is for example a composition of matter molecules that, when administered to a subject, induces an immune response. Vaccines can comprise polynucleotide molecules, polypeptide molecules, and carbohydrate molecules, as well as derivatives and combinations of each, such as glycoproteins, lipoproteins, carbohydrate-protein conjugates, fusions between two or more polypeptides or polynucleotides, and the like.

In some further embodiments, the isolated IA protein complex suitable as a vaccine comprising at least two recombinant proteins. The phrase “recombinant protein” includes proteins, in particular recombinant fusion proteins that are prepared, expressed, created or isolated by recombinant means, such as proteins expressed using a recombinant expression vector transfected into a host cell.

In some advantageous embodiments, the isolated IA protein complex suitable as a vaccine comprising at least two recombinant proteins. The term “recombinant fusion protein” refers in particular to a protein produced by recombinant technology which comprises segments i.e. amino acid sequences, from heterologous sources, such as different proteins, different protein domains or different organisms. The segments are joined either directly or indirectly to each other via peptide bonds. By indirect joining it is meant that an intervening amino acid sequence, such as a peptide linker is juxtaposed between segments forming the fusion protein. A recombinant fusion protein is encoded by a nucleotide sequence, which is obtained by genetically joining nucleotide sequences derived from different regions of one gene and/or by joining nucleotide sequences derived from two or more separate genes. These nucleotide sequences can be derived from P. falciparum, but they may also be derived from other organisms, the plasmids used for the cloning procedures or from other nucleotide sequences.

Furthermore, the encoding nucleotide sequences may be synthesized in vitro without the need for initial template DNA samples e.g. by oligonucleotide synthesis from digital genetic sequences and subsequent annealing of the resultant fragments. Desired protein sequences can be “reverse translated” e.g. using appropriate software tools. Due to the degeneracy of the universal genetic code, synonymous codons within the open-reading frame (i.e. the recombinant protein coding region) can be exchanged in different ways, e.g. to remove cis-acting instability elements (e.g. AUUUA), to remove, introduce or modify the secondary and tertiary mRNA structures (e.g. pseudoknots, stem-loops, . . . ), to avoid self-complementary regions that might trigger post-transcriptional gene silencing (PGTS), to change the overall AT:GC content, or to adjust the codon-usage to the expression host. Such changes can be designed manually or by using appropriate software tools or through a combination.

A recombinant fusion protein can be a recombinant product prepared using recombinant DNA methodology and expression in a suitable host cell, as is known in the art (see for example Sambrook et al., (2001) Molecular Cloning, Cold Spring Harbor Laboratory Press, Cold Spring Harbor, N. Y). Nucleotide sequences encoding specific isolated protein domain may be conveniently prepared, for example by polymerase chain reaction using appropriate oligonucleotide primers corresponding to the 5′ and 3′ regions of the domain required for isolation, and a full length coding of the isolated protein domain sequence as template. The source of the full length coding protein sequence may be for example, DNA extracted from Infectious agent/pathogen/pathogen cell or a plasmid vector containing a cloned full length gene. Alternatively, the protein coding sequence may partially or completely be synthesized in vitro or a combination of different approaches may be used.

In the context of the immunoassemblin/protein complexes according to the present disclosure, each of the fusion protein units may comprise an antigen. As used herein the terms “antigen” and “immunogen” are used herein interchangeably. However, according to the present disclosure both antigen and immunogen refers to a molecule that is capable of eliciting an immune response by an organism's immune system. Throughout the present disclosure, the term antigen will be used since it refers directly to the molecule that binds to the product of the immune response—the antibody. The antigens in the IA protein complexes are antigens capable of inducing an immune response. Suitable antigens may be for example identified by analyzing human blood samples from endemic regions vs non-related regional inhabitants screening for reactivity against recombinant produced promising literature known proteins. Antigens according to the present disclosure include any component, including variants, mutants, fragments thereof, being part of the proteome of the pathogen, in particular the antigen is derived from a surface protein or surface structure of the pathogen. The term “antigen” may include e.g. a cytokine, interleukin, interferon, toll-like receptor, peptide and an antibody variable domain.

For example, antigens according to the present disclosure are tumor antigens, auto-antigens, food allergens, antigens for example derived from a pathogen selected from the group of Chikungunya virus, Rabies virus, Streptococcus, Neisseria, Staphylococcus, Clostridiu, Trypanosoma, Leishmania, Salmonella, Flavivirus, Filiovirus and/or antigens derived from one or more pathogens responsible for an infectious disease deriving from Apicomplexan parasites like Malaria, Toxoplasmosis, and/or from viral infections like Ebola, HIV, HPV, Hepatitis, Tuberculosis, Zika, Influenza or Pertussis and/or deriving from diseases causing malignant tumors resulting in cancer. For example, an “antigens derived from Plasmodium falciparum surface protein” includes polypeptides comprising an amino acid sequence of the full-length Plasmodium falciparum surface protein or in particular only parts of the full-length protein, like specific domains or other parts.

As mentioned above, the antigens comprised in the different fusion proteins of the IA protein complex according to the present disclosure comprise different amino acid sequences i.e. said antigens of said recombinant fusion protein units differ in their amino acid sequence. In particular, the first antigen that is fused/inked N-terminal and/or C-terminal to at least one of the immunoglobulin heavy chain constant domains in the HC fusion polypeptide differs in the amino acid sequence from the second antigen that is linked N-terminal and/or C-terminal to the C_{L}-domain in the LC fusion polypeptide. Therefore, the antigens comprised in the fusion protein units of the IAs are different antigens comprising a different amino acid sequence.

In some advantageous embodiments, protein sequences of said antigens have a sequence identity of 99% or less, preferably 98% or less, more preferably of 95% or less, even more preferably of 90%, still more preferably of 80% or less and most preferably of 70% or less.

“Percent sequence identity”, with respect to two amino acid or polynucleotide sequences, refers to the percentage of residues that are identical in the two sequences when the sequences are optimally aligned. Thus, 80% amino acid sequence identity means that 80% of the amino acids in two optimally aligned polypeptide sequences are identical. Percent identity can be determined, for example, by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in “Atlas of Protein Sequence and Structure”, M. O. Dayhoff et., Suppl. 3:353-358, National Biomedical Research Foundation, Washington, D.C., which adapts the local homology algorithm of Smith and Waterman (1981) Advances in Appl. Math. 2:482-489 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters 5 recommended by the manufacturer and described in the Wisconsin Sequence Analysis Package referred to above. An example of an algorithm that is suitable for determining sequence similarity is the BLAST algorithm, which is described in Altschul, et al., J. Mol. Biol. 215:403-410 (1990). Software for performing BLAST analyses is publicly available through the National Center for Biotechnology Information (http://www.ncbi.nlm.nih.gov/).

In some advantageous embodiments, the protein complex according to the present disclosure comprises two or more variants of the same antigen that represent different strains, isolates, allelic variants, mutants and/or single nucleotide polymorphisms. It is a challenging fact that most of target antigens for the development of highly specific vaccines particularly exhibit polymorphisms which are often localized to B cell and T cell epitopes (Proietti and Doolan, 2014). These natural variants may originate from allelic polymorphisms (e.g. MSP1: all observed alleles are clearly divided into two allelic classes like MAD20 and Wellcome haplotypes), antigenic polymorphisms (multiple genetically stable alternative forms of antigen-coding genes originating from classical mutation/recombination events) or from the sequential expression of alternate forms of an antigen by the same clonal parasite lineage which leads to antigenic variation, e.g. PfEMP-1 and rifins (Roy et al., 2008). Undoubtedly, a second generation vaccine not only has to include multiple key antigens from the major life cycle stages, but to achieve the desired mimicry of natural strain-transcending acquired immunity, RTS,S successors must be effective against antigenic or allelic target variants as well, to ensure efficacy against all alternative circulating strains in the field (Moorthy and Kieny, 2010). Recent efforts to overcome antigenic diversity and allelic specificity for highly polymorphic vaccine candidate AMA1 demonstrated that combining four or five different AMA1 alleles (by testing a total of 108 immunogen-parasite combinations) is sufficient to cover the majority of naturally observed polymorphisms and to break strain-specific barriers in vitro (see Proietti, C. and D. L. Doolan (2014). “The case for a rational genome-based vaccine against malaria.” Front Microbiol 5: 741, Roy, S. W. et al., (2008). “Evolution of allelic dimorphism in malarial surface antigens.” Heredity (Edinb) 100(2): 103-110, Moorthy, V. S. and M. P. Kieny (2010). “Reducing empiricism in malaria vaccine design.” Lancet Infect Dis 10(3): 204-211, Miura, K. et al., (2013). “Overcoming allelic specificity by immunization with five allelic forms of Plasmodium falciparum apical membrane antigen 1.” Infect Immun 81(5): 1491-1501.).

Furthermore, in some embodiments the protein complex according to the present disclosure comprises antigens derived from two different pathogens.

Preferably, the antigens comprised the protein complex according to the present disclosure are antigens derived from an Apicomplexan parasite. The Apicomplexa (also referred to as Apicomplexia) are a large group of protists, most of which possess a unique organelle called apicoplast and an apical complex structure involved in penetrating a host's cell. They are a diverse group including organisms such as coccidia, gregarines, piroplasms, haemogregarines, and plasmodia (P. falciparum, Plasmodium vivax, Plasmodium ovale, Plasmodium malariae, Plasmodium knowlesi). Diseases caused by apicomplexan organisms include, but are not limited to Babesiosis (Babesia), Malaria (Plasmodium), Coccidian diseases including Cryptosporidiosis (Cryptosporidium parvum), Cyclosporiasis (Cyclospora cayetanensis), Isosporiasis (Isospora belli) and Toxoplasmosis (Toxoplasma gondii). For example, antigens comprised the protein complex according to the present disclosure includes antigens derived from cellular surface structures presented on the surface of the parasite of the phylum Apicomplexa.

Apicomplexa surface structures and/or surface proteins are preferably membrane-bound or associated proteins or proteins known to be secreted. These proteins can e.g. be identified by analyzing the Genome or known genes for the presence of an N-terminal signal peptide, the presence of a PEXEL motif, the presence of a GPI anchor motif, or the presence of one or more transmembrane domains using generally available software tools. These proteins and their homologues e.g. include but are not limited to:
CelTOS (cell traversal protein for ookinetes and sporozoites), Antigen 2 (PfAg2, PvAg2, PoAg2, etc.)
CSP (circumsporozoite protein)
EBA175 (Erythrocyte binding antigen 175)
EXP1 (Exported Protein 1); synonyms: CRA1 (Circumsporozoite-Related Antigen-1/Cross-Reactive Antigen-1), AG 5.1 (Exported antigen 5.1), QF119
MSP1 (Merozoite surface protein 1); synonyms: MSA1 (Merozoite surface antigen 1), PMMSA, p190, p195, gp190, gp195
MSP2 (Merozoite surface protein 2);
MSP3 (Merozoite surface protein 3); synonym: SPAM (secreted polymorphic antigen associated with the merozoite)
MSP4 (Merozoite surface protein 4)
MSP5 (Merozoite surface protein 5)
MSP7 (Merozoite surface protein 7)
MSP8 (Merozoite surface protein 8)
MSP9 (Merozoite surface protein 9)
MSP10 (Merozoite surface protein 10)
MTRAP (merozoite TRAP homologue, merozoite TRAP homolog, merozoite TRAP-like protein)
Pf38; synonym: 6-cysteine protein
Rh2a (Reticulocyte binding protein 2 homolog a)
Rh2b (Reticulocyte binding protein 2 homologue b)
Rh4 (Reticulocyte binding protein homologue 4)
Rh5 (Reticulocyte binding protein homologue 5)
Ripr, PfRipr (Rh5 interacting protein)
Ron2 (rhoptry neck protein 2)
Ron4 (rhoptry neck protein 4)
Ron5 (rhoptry neck protein 5)
Ron6 (rhoptry neck protein 6)
TRAMP (thrombospondin-related apical membrane protein); synonym: PTRAMP
TRAP (Thrombospondin-related anonymous protein); synonym: SSP2 (Sporozoite Surface Protein 2)
AMA1 (apical membrane antigen 1)
GLURP (Glutamine-rich protein)
RhopH2 (High Molecular Weight Rhoptry Protein-2)
RhopH3 (High Molecular Weight Rhoptry Protein-3)

Malarial diseases in humans are caused for example by five species of the Plasmodium parasite: P. falciparum, P. vivax, P. ovale, P. malariae and P. knowlesi, wherein the most prevalent being Plasmodium falciparum and Plasmodium vivax. Malaria caused by Plasmodium falciparum (also called malignantor malaria, falciparum malaria or malaria tropica) is the most dangerous form of malaria, with the highest rates of complications and mortality. Almost all malarial deaths are caused by P. falciparum.

Therefore, some advantageous embodiments, the antigens comprised the IA protein complex according to the present disclosure are antigens derived from at least one Plasmodium parasite selected from the group consisting of P. falciparum, P. vivax, P. ovale, P. knowlesi and P. malariae. In particular, the antigens comprised the protein complex according to the present disclosure are antigens derived from P. falciparum. In some embodiments, the antigens comprised in the protein complex according to the present disclosure are antigens comprising or consisting of an amino acid sequence selected from the group consisting of SEQ ID NO. 1 to 31.

Furthermore, the protein complex as well as the compositions according to the present disclosure are suitable as human and/or animal vaccines against a pathogen, in particular against a parasite of the genus Plasmodium including P. falciparum, Plasmodium vivax, Plasmodium malariae and/or Plasmodium ovale. In an advantageous embodiment, the parasite is P. falciparum.

Advantageous antigens, in particular comprised or consisted in the fusion proteins of the protein complexes according to the present disclosure suitable as human vaccines against Plasmodium falciparum are listed in the following Table 1.

TABLE 1
Single or multi-domain proteins for P. falciparum vaccines

| SEQ ID | Domain(s) | Sequence |
|---|---|---|
| 1 | PfMSP1₁₉ (strain | ISQHQCVKKQCPENSGCFRHLDEREECKCLLNYKQEGDKCVENP |
| | 3D7/MAD20, aa | NPTCNENNGGCDADATCTEEDSGSSRKKITCECTKPDSYPLFDGI |
| | 1608-1702) | FCSSSN |
| 2 | PfMSP1₁₉ (strain | ISQHQCVKKQCPENSGCFRHLDEREECKCLLNYKQEGDKCVENP |
| | FUP/Palo Alto, aa | NPTCNENNGGCDADAKCTEEDSGSNGKKITCECTKPDSYPLFDGI |
| | 1608-1702) | FCSSSN |
| 3 | PfMSP1₁₉ (strain | ISQHQCVKKQCPQNSGCFRHLDEREECKCLLNYKQEGDKCVENP |
| | Wellcome/K1, aa | NPTCNENNGGCDADAKCTEEDSGSNGKKITCECTKPDSYPLFDGI |
| | 1608-1702) | FCSSS |
| 4 | PfMSP1₁₉ (strain | ISQHQCVKKQCPQNSGCFRHLDEREECKCLLNYKQEGDKCVENP |
| | Type2/Thai, aa | NPTCNENNGGCDADAKCTEEDSGSNGKKITCECTKPDSYPFFDGI |
| | 1608-1702) | FCSSSN |
| 5 | Tetra_MSP1₁₉ | ISQHQCVKKQCPENSGCFRHLDEREECKCLLNYKQEGDKCVENP |
| | (strain- | NPTCNENNGGCDADATCTEEDSGSSRKKITCECTKPDSYPLFDGI |
| | transcending) | FCSSSNAAVAMAISQHQCVKKQCPENSGCFRHLDEREECKCLLNY |
| | | KQEGDKCVENPNPTCNENNGGCDADAKCTEEDSGSNGKKITCEC |
| | | TKPDSYPLFDGIFCSSSNAAVAMAISQHQCVKKQCPQNSGCFRHL |
| | | DEREECKCLLNYKQEGDKCVENPNPTCNENNGGCDADAKCTEED |
| | | SGSNGKKITCECTKPDSYPLFDGIFCSSSNAAVAMAISQHQCVKKQ |
| | | CPQNSGCFRHLDEREECKCLLNYKQEGDKCVENPNPTCNENNGG |
| | | CDADAKCTEEDSGSNGKKITCECTKPDSYPFFDGIFCSSSN |
| 6 | Pfs25_FKO (3D7, | VTVDTVCKRGFLIQMSGHLECKCENDLVLVNEETCEEKVLKCDEKT |
| | aa 24-193) | VNKPCGDFSKCIKIDGNPVSYACKCNLGYDMVNNVCIPNECKNVA |
| | | CGNGKCILDTSNPVKTGVCSCNIGKVPNVQDQKCSKDGETKCSLK |
| | | CLKENEACKAVDGIYKCDCKDGFIIDNEASICT |
| 7 | Pfs25_SHKO (3D7 | VTVDTVCKRGFLIQMSGHLECKCENDTVLVNEETCEEKVLKCDEK |
| | aa 24-193) | TVNKPCGDFSKCIKIDGNPVSYACKCNLGYDMVNNVCIPNECKNVT |
| | | CGNGKCILDTSNPVKTGVCSCNIGKVPNVQDQKCSKDGETKCSLK |
| | | CLKENETCKAVDGIYKCDCKDGFIIDNEASICT |
| 8 | Pfs28 (3D7, aa 24-191) | VTENTICKYGYLIQMSNHYECKCIEGYVLINEDTCGKKVVCDKVEN |
| | | SFKACDEYAYCFDLGNKNNEKQIKCMCRTEYTLTAGVCVPNVCRD |
| | | KVCGKGKCIVDPANSLTHTCSCNIGTILNQNKLCDIQGDTPCSLKCA |
| | | ENEVCTLEGNYYTCKEDPSSNGGGNTVDQA |
| 9 | PfCSP_TSR (3D7, | PSDKHIEQYLKKIQNSLSTEWSPCSVTCGNGIQVRIKPGSANKPKD |
| | aa 311-384) | ELDYENDIEKKICKMEKCSSVFNVVNSS |
| 10 | PfTRAP_TSR (3D7, | EKTASCGVWDEWSPCSVTCGKGTRSRKREILHEGCTSELQEQCE |
| | aa 239-289) | EERCLPK |
| 11 | PfMTRAP_TSR | THDTCDEWSEWSACTHGISTRKCLSDSSIKDETLVCTKCDKWGE |
| | (3D7, aa 25-98) | WSECKDGRMHRKVLNCPFIKEEQECDVNNE |
| 12 | PfTRAMP_TSR | FYSEWGEWSNCAMDCDHPDNVQIRERECIHPSGDCFKGDLKESR |
| | (3D7, aa 244-307)) | PCIIPLPCNELFSHKDNSAFK |
| 13 | PfCTMT (3D7) | PSDKHIEQYLKKIQNSLSTEWSPCSVTCGNGIQVRIKPGSANKPKD |
| | (stage- | ELDYENDIEKKICKMEKCSSVFNVVNSSAAVAMAEKTASCGVWDE |
| | transcending) | WSPCSVTCGKGTRSRKREILHEGCTSELQEQCEEERCLPKAAVA |
| | | MATHDTCDEWSEWSACTHGISTRKCLSDSSIKDETLVCTKCDKWG |
| | | EWSECKDGRMHRKVLNCPFIKEEQECDVNNEAAVAMAFYSEWGE |
| | | WSNCAMDCDHPDNVQIRERECIHPSGDCFKGDLKESRPCIIPLPPC |
| | | NELFSHKDNSAFK |
| 14 | PvTRAP_TSR (Sal- | ERVANCGPWDPWTACSVTCGRGTHSRSRPSLHEKCTTHMVSEC |
| | 1, aa 235-297) | EEGECPVEPEPLPVPAPLPT |
| 15 | PkTRAP_TSR | EVERIAKCGPWDDWTPCSVTCGKGTHSRSRPLLHAGCTTHMVKE |
| | (strain H, aa 235-286) | CEMDECPVEP |
| 16 | MS_TRAP-TSRs | EKTASCGVWDEWSPCSVTCGKGTRSRKREILHEGCTSELQEQCE |
| | (species- | EERCLPKAAVAMAERVANCGPWDPWTACSVTCGRGTHSRSRPS |
| | transcending) | LHEKCTTHMVSECEEGECPVEPEPLPVPAPLPTAAVAMAEVERIAK |
| | | CGPTAATCGGCCGTGGCCATGGCTWDDWTPCSVTCGKGTHSRS |
| | | RPLLHAGCTTHMVKECEMDECPVEP |
| 17 | PfMSP3A_Nterm | SKEIVKKYNLNLRNAILNNNSQIENEENVNTTITGNDFSGGEFLWPG |
| | (3D7, aa 25-354) | YTEELKAKKASEDAEKAANDAENASKEAEEAAKEAVNLKESDKSY |
| | | TKAKEAATAASKAKKAVETALKAKDDAEKSSKADSISTKTKEYAEK |
| | | AKNAYEKAKNAYQKANQAVLKAKEASSYDYILGWEFGGGVPEHKK |
| | | EENMLSHLYVSSKDKENIAKENDDVLDEKEEEAEETEEEELEEKNE |
| | | EETESEISEDEEEEEEEEKEEENDKKKEQEKEQSNENNDQKKDME |
| | | AQNLISKNQNNNEKNVKEAAESIMKTLAGLIKGNNQIDSTLKDLVEE |
| | | LSKYFKNH |
| 18 | PfMSP3B_Nterm | SKEIVKKYNLNLRNAILNNNSQIENEENDIKYELNEQNDENVNTPIV |
| | (3D7, aa 25-354) | GNMEFGEGFSADDQKDIEAYKKAKQASQDAEQAAKDAENAAKDA |
| | | EEAAKDAEKLKESDESYTKAKEACTAASKAKKAVETALKAKDDAET |
| | | ALKTSETPEKPSRINLFSRKTKEYAEKAKNAYEKAKNAYQKANQAV |
| | | LKAKEASSYDYILGWEFGGGVPEHKKEENMLSHLYVSSKDKENIAK |
| | | ENDDVLDEKEEEAEETEEEELEEKNEEETESEISEDEEEEEEEEKE |
| | | EENDKKKEQEKEQSNENNDQKKDMEAQNLISKNQNNNEKNVKEA |
| | | AESIMKTLAGLIKGNNQIDSTLKDLVEELSKYFKNH |
| 19 | PfMSP6₃₆ (3D7, aa | NGLTGATENIAQVVQANSETNKNPTSHSNSTTTSLNNNILGWEFG |
| | 144-369) | GGAPQNGAAEDKKTEYLLEQIKIPSWDRNNIPDENEQVIEDPQEDN |
| | | KDEDEDEETETENLETEDDNNEEIEENEEDDIDEESVEEKEEEEEK |
| | | KEEEEKKEEKKEEKKPDNEITNEVKEEQKYSSPSDINAQNLISNKN |
| | | KKNDETKKTAENIVKTLVGLFNEKNEIDSTINNLVQEMIHLFS |
| 20 | PfMSP7₂₂ (3D7, aa | SETDTQSKNEQEISTQGQEVQKPAQGGESTFQKDLDKKLYNLGDV |
| | 177-350) | FNHVVDISNKENKINLDEHDKKYTDFKKEYEDFVLNSKEYDIIKNLII |
| | | MFGQEDNKAKNGKTDIVSEAKHITEIFIKLFKDKEYHEQFKNYIYGV |
| | | YSYAKQNSHLSEKKIKQEEEYKKFLEYSFNLLNT |
| 21 | PfExp1/PfCra1 | EKTNKGTGSGVSSKKKNKKGSGEPLIDVHDLISDMIKKEEELVEVN |
| | (3D7, aa 23-79 + aa | KRKSKYKLATSNTEKGRHPFKIGSSDPADNANPDADSESNGEPNA |
| | 102-162, w/o TMD | DPQVTAQDVTPEQPQGDDNNLVSGPEH |
| | incl. McAb 5.1 | |
| | epitope in bold | |
| 22 | PfEBA175_F2 | DKNSVDTNTKVWECKKPYKLSTKDVCVPPRRQELCLGNIDRIYDK |
| | (3D7, aa 463-745) | NLLMIKEHILAIAIYESRILKRKYKNKDDKEVCKIINKAFADIRDIIGGT |
| | | DYWNDLSNRKLVGKINTNSNYVHRNKQNDKLFRDEWWKVIKKDV |
| | | WNVISWVFKDKTVCKEDDIENIPQFFRWFSEWGDDYCQDKTKMIE |
| | | TLKVECKEKPCEDDNCKRKCNSYKEWISKKKEEYNKQAKQYQEY |
| | | QKGNNYKMYSEFKSIKPEVYLKKYSEKCSNLNFEDEFKEELHSDYK |
| | | NKCTMCPEV |
| 23 | PfAMA1_GKO | IEIVERSNYMGNPWTEYMAKYDIEEVHGSGIRVDLGEDAEVAGTQY |
| | (3D7; aa 97-546) | RLPSGKCPVFGKGIIIENSNTAFLTPVATGNQYLKDGGFAFPPTEPL |
| | | MSPMTLDEMRHFYKDNKYVKNLDELTLCSRHAGNMIPDNDKNSN |
| | | YKYPAVYDDKDKKCHILYIAAQENNGPRYCNKDESKRNSMFCFRP |
| | | AKDISFQNYAYLSKNVVDNWEKVCPRKNLQNAKFGLWVDGNCEDI |
| | | PHVNEFPAIDLFECNKLVFELSASDQPKQYEQHLTDYEKIKEGFKN |
| | | KNAAMIKSAFLPTGAFKADRYKSHGKGYNWGNYNTETQKCEIFNV |
| | | KPTCLINNAAYIATTALSHPIEVENNFPCSLYKDEIMKEIERESKRIKL |
| | | NDNDDEGNKKIIAPRIFISDDKDSLKCPCDPEMVSNSACRFFVCKC |
| | | VERRAEVTSNNEVVVKEEYKDEYADIPEHKPTYDKMK |
| 24 | PfRON2L (3D7, aa | MDITQQAKDIGAGPVASCFTTRMSPPQQICLNSVVNTALSTSTQSA |
| | 2020-2067) | MK |
| 25 | PfRh2A₁₅ (3D7, aa | KKYETYVDMKTIESKYTTVMTLSEHLLEYAMDVLKANPQKPIDPKA |
| | 446-558) | NLDSEVVKLQIKINEKSNELDNAASQVKTLIIIMKSFYDIIISEKASMD |
| | | EMEKKELSLNNYIEKTDY |
| 26 | PfCyRPA (3D7, aa | DSRHVFIRTELSFIKNNVPCIRDMFFIYKRELYNICLDDLKGEEDETH |
| | 29-345) | IYVQKKVKDSWITLNDLFKETDLTGRPHIFAYVDVEEIIILLCEDEEFS |
| | | NRKKDMTCHRFYSNDGKEYNNSEITISDYILKDKLLSSYVSLPLKIE |
| | | NREYFLICGVSPYKFKDDNKKDDILCMASHDKGETWGTKIVIKYDN |
| | | YKLGVQYFFLRPYISKNDLSFHFYVGDNINNVKNVNFIECTHEKDLE |
| | | FVCSNRDFLKDNKVLQDVSTLNDEYIVSYGNDNNFAECYIFFNNEN |
| | | SILIKPEKYGNTTAGCYGGTFVKIDENRTLFIYS |
| 27 | PfSEA1 (3D7, aa | NEDRGIYDELLENDMCDLYNLKMHDLHNLKSYDFGLSKDLLKKDIFI |
| | 811-1083) | YSNNLKNDDMDDDDNNNMNDIAIGENVIYENDIHENNIDDNDMYN |
| | | NYVNGNDLYINNMQDDAMDDIVYDEEEIKSFLDKLKSDISNQMNVK |
| | | NGNVEVTGNGGNEEMSYINNDENLQAFDLLDNFHMDDYGNNYND |
| | | NEEDGDGDGDDDEQKKRKQKELHNVNGKLDLSDLNELNVDDINN |
| | | NFYMSTPRKSIDERKDTECQTDFPLLDVSRNTDRTPRRKSVEVILV |
| | | EAAAHHHHHHSEKDEL |
| 28 | PfAMA1-DiCo1 | QNYWEHPYQKSDVYHPINEHREHPKEYEYPLHQEHTYQQEDSGE |
| | (strain- | DENTLQHAYPIDHEGAEPAPQEQNLFSSIEIVERSNYMGNPWTEY |
| | transcending) | MAKYDIEEVHGSGIRVDLGEDAEVAGTQYRLPSGKCPVFGKGIIIEN |
| | | SQTTFLTPVATENQDLKDGGFAFPPTKPLMSPMTLDQMRHFYKDN |
| | | EYVKNLDELTLCSRHAGNMNPDNDKNSNYKYPAVYDDKDKKCHIL |
| | | YIAAQENNGPRYCNKDESKRNSMFCFRPAKDKSFQNYVYLSKNVV |
| | | DNWEKVCPRKNLENAKFGLWVDGNCEDIPHVNEFSANDLFECNK |
| | | LVFELSASDQPKQYEQHLTDYEKIKEGFKNKNADMIRSAFLPTGAF |
| | | KADRYKSHGKGYNWGNYNRKTQKCEIFNVKPTCLINDKSYIATTAL |
| | | SHPIEVEHNFPCSLYKDEIKKEIERESKRIKLNDNDDEGNKKIIAPRIF |
| | | ISDDKDSLKCPCDPEIVSQSTCNFFVCKCVEKRAEVTSNNEVVVKE |
| | | EYKDEYADIPEHKPTYDK |
| 29 | PfAMA1-DiCo2 | QNYWEHPYQKSDVYHPINEHREHPKEYEYPLHQEHTYQQEDSGE |
| | (strain- | DENTLQHAYPIDHEGAEPAPQEQNLFSSIEIVERSNYMGNPWTEY |
| | transcending) | MAKYDIEEVHGSGIRVDLGEDAEVAGTQYRLPSGKCPVFGKGIIIEN |
| | | SQTTFLKPVATGNQDLKDGGFAFPPTNPLISPMTLNGMRDFYKNN |
| | | EYVKNLDELTLCSRHAGNMNPDNDENSNYKYPAVYDYNDKKCHIL |
| | | YIAAQENNGPRYCNKDESKRNSMFCFRPAKDKLFENYVYLSKNVV |
| | | HNWEEVCPRKNLENAKFGLWVDGNCEDIPHVNEFSANDLFECNK |
| | | LVFELSASDQPKQYEQHLTDYEKIKEGFKNKNADMIRSAFLPTGAF |
| | | KADRYKSRGKGYNWGNYNRKTQKCEIFNVKPTCLINDKSYIATTAL |
| | | SHPIEVENNFPCSLYKNEIMKEIERESKRIKLNDNDDEGNKKIIAPRI |
| | | FISDDKDSLKCPCDPEMVSQSTCRFFVCKCVERRAEVTSNNEVVV |
| | | KEEYKDEYADIPEHKPTYDN |
| 30 | PfAMA1-DiCo3 | QNYWEHPYQKSDVYHPINEHREHPKEYEYPLHQEHTYQQEDSGE |
| | (strain- | DENTLQHAYPIDHEGAEPAPQEQNLFSSIEIVERSNYMGNPWTEY |
| | transcending) | MAKYDIEEVHGSGIRVDLGEDAEVAGTQYRLPSGKCPVFGKGIIIEN |
| | | SKTTFLTPVATENQDLKDGGFAFPPTEPLMSPMTLDDMRDLYKDN |
| | | KYVKNLDELTLCSRHAGNMIPDNDKNSNYKYPAVYDYEDKKCHILY |
| | | IAAQENNGPRYCNKDQSKRNSMFCFRPAKDISFQNYVYLSKNVVD |
| | | NWEKVCPRKNLQNAKFGLWVDGNCEDIPHVNEFSAIDLFECNKLV |
| | | FELSASDQPKQYEQHLTDYEKIKEGFKNKNADMIRSAFLPTGAFKA |
| | | DRYKSHGKGYNWGNYNTETQKCEIFNVKPTCLINDKSYIATTALSH |
| | | PNEVEHNFPCSLYKDEIKKEIERESKRIKLNDNDDEGNKKIIAPRIFIS |
| | | DDIDSLKCPCAPEIVSQSTCNFFVCKCVEKRAEVTSNNEVVVKEEY |
| | | KDEYADIPEHKPTYDK |
| 31 | PfRh5_GKO (3D7, | FENAIKKTKNQENNLALLPIKSTEEEKDDIKNGKDIKKEIDNDKENIK |
| | aa 25-526) | TNNAKDHSTYIKSYLNTNVNDGLKYLFIPSHNSFIKKYSVFNQINDG |
| | | MLLNEKNDVKNNEDYKNVDYKNVNFLQYHFKELSNYNIANSIDILQ |
| | | EKEGHLDFVIIPHYTFLDYYKHLSYNSIYHKSSTYGKCIAVDAFIKKIN |
| | | EAYDKVKSKCNDIKNDLIATIKKLEHPYDINNKNDDSYRYDISEEIDD |
| | | KSEETDDETEEVEDSIQDTDSNHAPSNKKKNDLMNRAFKKMMDEY |
| | | NTKKKKLIKCIKNHENDFNKICMDMKNYGTNLFEQLSCYNNNFCNT |
| | | NGIRYHYDEYIHKLILSVKSKNLNKDLSDMTNILQQSELLLTNLNKK |
| | | MGSYIYIDTIKFIHKEMKHIFNRIEYHTKIINDKTKIIQDKIKLNIWRTFQ |
| | | KDELLKRILDMSNEYSLFITSDHLRQMLYNTFYSKEKHLNNIFHHLIY |
| | | VLQMKFNDVPIKMEYFQTYKKNKPLTQ |

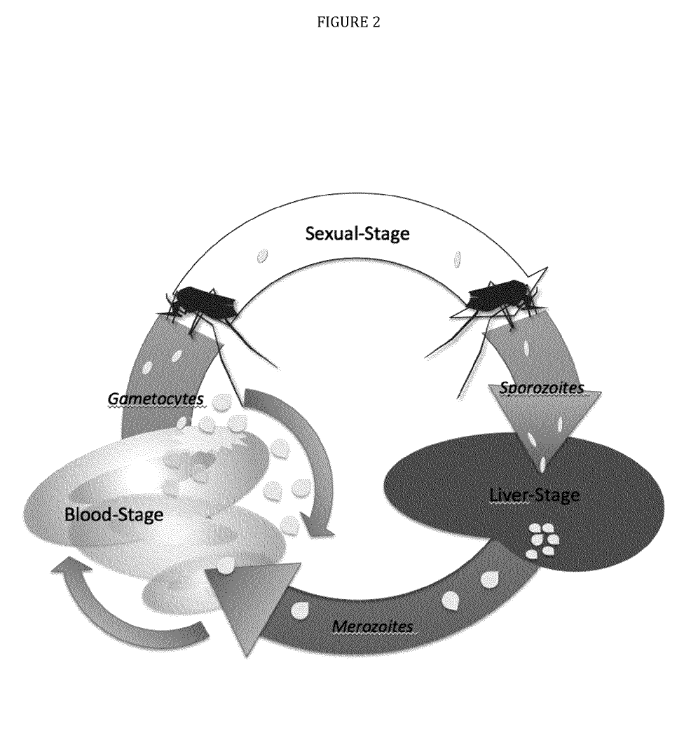

Briefly, the plasmodial life cycle (FIG. 2) in man starts with the inoculation of a few sporozoites through the bite of an Anopheles mosquito. Within minutes, sporozoites invade the hepatocyte and start their development, multiplying by schizogony (liver stage or pre-erythrocytic stage). After a period of 5-14 days—depending on the plasmodial species—schizonts develop into thousands of merozoites that are freed into the bloodstream and invade the red blood cells (RBCs), initiating the blood stage. In the RBC, each merozoite develops into a trophozoite that matures and divides, generating a schizont that, after fully matured, gives rise to up to 32 merozoites within 42-72 h, depending on the plasmodial species. The merozoites, released into the bloodstream, will invade other RBC, maintaining the cycle. Some merozoites, after invading a RBC, develop into sexual forms—the male or female gametocytes which also enter the bloodstream after maturation and erythrocyte rupture. If a female Anopheles mosquito takes its blood meal and ingests the gametocytes, it will become infected and initiates the sexual stage of the Plasmodium life cycle. In the mosquito gut, the male gametocyte fuses with the female gametocyte, forming the ookinete, which binds to and passes through the gut wall, remains attached to its external face and transforms into the oocyst. The oocyst will divide by sporogony, giving rise to thousands of sporozoites that are released in the body cavity of the mosquito and eventually migrate to its salivary gland, where they will maturate, becoming capable of starting a new infection in humans when the mosquito bites the host for a blood meal.

In some embodiments, the antigens comprised in the protein complex according to the present disclosure are antigens are derived from at least two different proteins presented on the surface of a parasite. In particular, the antigens are derived from at least two different proteins presented on the surface of said parasite in at least two different life cycle main stages of said parasite.

In some advantageous embodiments, the protein complex according to the present disclosure comprises antigens derived from cellular surface structures, wherein the cellular surface structures are presented on the surface of the parasite in at least one main stage of the Apicomplexa life cycle stages like the pre-erythrocytic stage, the sexual stage and/or the blood stage. Preferably, the P. falciparum surface protein is presented on the surface of the parasite in the pre-erythrocytic stage and/or the blood stage.

### The Pre-Erythrocytic Main Stage:

### a) Sporozoite

The sporozoite remains in the bloodstream for a very short period of time before invading a hepatocyte. Examples for Plasmodium protein antigens expressed in the sporozoite are the circumsporozoite protein (CSP), the major constituent of the outer membrane of the sporozoite (Nussenzweig and Nussenzweig., 1989). Induced antibodies may be able to block the binding and the entrance of the sporozoite into the hepatocyte.

### b) Liver Stage

During this stage, immunity is mostly mediated by cellular-dependent mechanisms involving CD8+ T cells, CD4+ T cells, natural killer (NK) cells and γδ T cells. CSP is expressed both in the sporozoite and during the liver stage. So, much of the research involving CSP has switched from the immunodominant repeats inducing humoral response to regions that are able to induce cytotoxic T-cell responses. Other identified liver-stage antigens include liver-stage antigen-1 (LSA-1), LSA-2, LSA-3, SALSA and STARP, among others (Garcia, Puentes et al. 2006)

### The Asexual Blood Main Stage:

### c) Merozoite

Besides the sporozoite, the merozoite is the only stage in the human host in which the malaria parasite is extracellular. In contrast to the sporozoite, several cycles of merozoite release will occur during a malaria infection, making them often available. A major ligand in P. falciparum is the erythrocyte-binding antigen-175 (EBA-175), located in the microneme (Sim, Toyoshima et al. 1992). Several merozoite surface proteins (MSPs) have been identified, but for most of them their function still has to be further elucidated. In the case of the major MSP, named MSP-1, a role has been postulated in merozoite binding to the RBC and in the subsequent biochemical mechanisms involved in invasion. This protein is synthesized as a precursor of 185-210 kDa in the late schizont stage and is processed to generate several polypeptides of varied molecular weights. A 42 kDa polypeptide (MSP1-42) is kept attached to the merozoite membrane, and it is further processed to generate a 19 kDa polypeptide (MSP1-19), which goes into the host cell. Besides MSP-1, at least eight other MSPs have been described in P. falciparum: MSP-2, MSP-3, MSP-4, MSP-5, MSP-6, MSP-7, MSP-8 and MSP-10. Another merozoite surface-associated antigen is the acidic-basic repeat antigen (ABRA). Proteins located in merozoite apical organelles have also been identified (e.g. the rhoptry proteins apical membrane antigen-1 (AMA-1), rhoptry-associated protein-1 (RAP-1) and RAP-2).

### d) Infected RBC

Once it has invaded the RBC, the parasite is supposed to have found a safer place to stay. One of the most studied molecules is the ring erythrocyte surface antigen (RESA). Further, the serine-rich protein (SERP or SERA) is a soluble protein expressed in the schizont stage and secreted in the parasitophorous vacuole. Other proteins that are located on the RBC membrane are the erythrocyte membrane protein-1 (EMP-1), EMP-2 and EMP-3. PfEMP-1, which binds to the receptors such as CD36 in the endothelium, is a family of proteins encoded by the so-called var genes.

In some embodiments, component A has a binding activity either for cellular surface structures presented on the surface of a parasite of the phylum Apicomplexa or for parasitic antigens presented on a parasitized host cell.

### The Sexual Main Stage:

### e) Sporogonic Cycle

Other Plasmodium protein antigens are expressed in sexually differentiated parasite stages such as Ps25, Ps28, Ps48/45 or Ps230. Antibodies against these sexual stage proteins may block the development of the parasite in mosquitoes.

In an advantageous embodiment, each of the heat stable fragments are from different Plasmodium surface proteins expressed in at least two different stages of the Plasmodium life cycle.

In advantageous embodiments, the heat stable fragments are selected from the group consisting of heat stable fragments comprising an EGF-like domain from MSP1, MSP4, MSP8, MSP10, PfRipr and Pfs25.

In further advantageous embodiments, the heat stable fragments are selected from the group consisting of heat stable fragments comprising a TSR domain is selected from CSP, MTRAP, TRAP and TRAMP.

In other advantageous embodiments, the heat stable fragments are selected from the group consisting of heat stable fragments from Pfs230, Pfs45/48, CelTos and Ron2, MSP1₁₉ and EXP1.

As mentioned above, in some advantageous embodiments the antigens are derived from the parasite is P. falciparum and the antigens are derived from cellular surface proteins presented on the surface of the parasite in the pre-erythrocytic main stage and the blood main stage.

In another advantageous embodiments the antigens are derived from at least three different proteins presented on the surface of a parasite. In particular, the antigens are derived from at least three different proteins presented on the surface of the parasite in at least three different life cycle main stages of the parasite. In particular, the parasite is P. falciparum and the different life cycle main stages are pre-erythrocytic stage, the blood stage and the sexual stage. Examples for the three antigens are Pfs25 FKO, AMA1 GKO and CSP_TSR GKO.

According to the present disclosure, antigens may be linked N-terminal and/or C-terminal in/to a fusion protein (e.g. at least one of the immunoglobulin heavy chain constant domains and N-terminal and/or C-terminal to the C_{L}-domain) in a protein complex of the present disclosure.

As mentioned above, “Linked” refers to non-covalent or covalent bonding between two or more molecules. Linking may be direct or indirect. Two molecules are indirectly linked when the two molecules are linked via a connecting molecule (linker). Two molecules are directly linked when there is no intervening molecule linking them. As mentioned above, the fusion protein units are linked either directly or indirectly to each other, preferably via peptide bonds or disulfide bonds. An example of indirect covalent linking is that an intervening amino acid sequence, such as a peptide linker is juxtaposed between segments forming the fusion protein. “Linked” according to the present disclosure includes in particular also that one protein is fused to another protein resulting in a fusion protein or fusion protein unit. Therefore, preferably the antigens are fused N-terminal and/or C-terminal to at least one of the immunoglobulin heavy chain constant domains and N-terminal and/or C-terminal to the C_{L}-domain in a fusion protein unit of the IA protein complex according to the present disclosure.

In some further embodiments or the present disclosure, the fusion proteins may be covalently or non-covalently linked to each other in the protein complex of the present disclosure. According to the present disclosure the term “covalently linked” comprises a covalent bond that involves the sharing of electron pairs between atoms. These electron pairs are known as shared pairs or bonding pairs and the stable balance of attractive and repulsive forces between atoms when they share electrons is known as covalent bonding. The term “non-covalently linked” comprises a non-covalent interaction that differs from a covalent bond in that it does not involve the sharing of electrons, but rather involves more dispersed variations of electromagnetic interactions between molecules or within a molecule. In some advantageous embodiments of the present disclosure, the fusion proteins are covalently linked to each other by a disulfide bond.

In some advantageous embodiments, the first fusion protein unit (HC unit 1) and the second fusion protein unit (LC unit 1) are covalently or non-covalently linked to each other, in particular the first and second fusion protein units are covalently linked to each other by a disulfide bond.

As mentioned above, in some advantageous embodiments the IA protein complexes according to the present disclosure comprise a third recombinant fusion protein unit comprising the immunoglobulin heavy chain constant domains C_{H}1 and C_{H}3 and a third antigen, wherein said third antigen is fused N-terminal and/or C-terminal to the immunoglobulin heavy chain constant domains of said third fusion protein (second HC fusion polypeptide unit 2, HC unit 2).

In some advantageous embodiments, the first fusion protein unit (HC unit 1) and the third fusion protein unit (HC unit 2) are covalently or non-covalently linked to each other, in particular the first and third fusion protein units are covalently linked to each other by a disulfide bond.

In another advantageous embodiment, the IA protein complexes according the present disclosure, comprise a fourth recombinant fusion protein unit comprising an immunoglobulin light chain constant domain CL, and a fourth antigen, wherein said fourth antigen is fused N- or C-terminal to the CL-domain (second LC fusion polypeptide unit 2, LC unit 2).

In some advantageous embodiments, the third fusion protein unit (HC unit 2) and the fourth fusion protein unit (LC unit 2) are covalently or non-covalently linked to each other, in particular the third and fourth fusion protein units are covalently linked to each other by a disulfide bond.

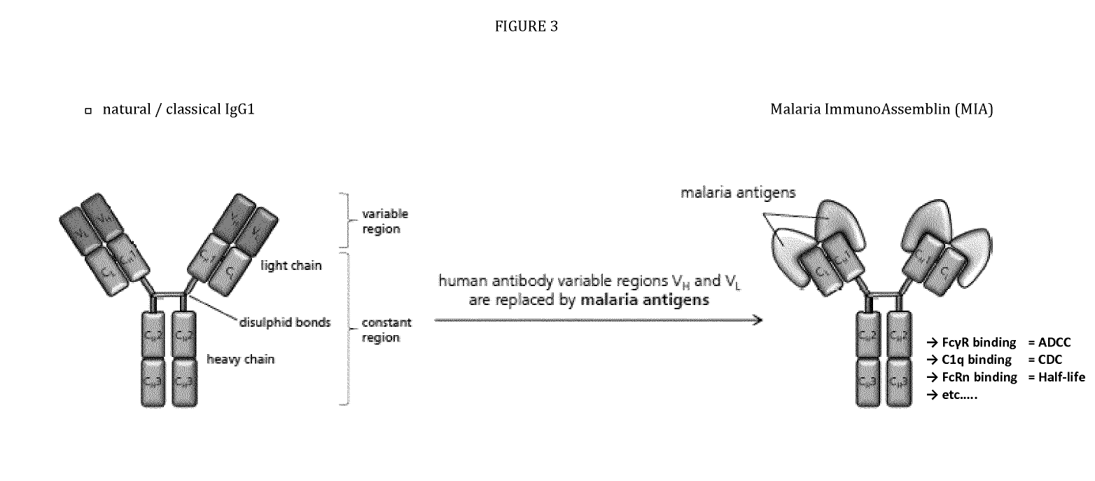

In particular, some advantageous embodiments pertains to IA protein complexes, wherein the first fusion protein (HC unit 1) and the second fusion protein (LC unit 1) are linked to each other, the third fusion protein (HC unit 2) and the fourth fusion protein (LC unit 2) are linked to each other, and the first fusion protein (HC unit 1) and the third fusion protein (HC unit 2) are linked to each other (see for example FIG. 3). This construct may be called an antibody-like protein complex. In particular, fusion protein units are covalently linked to each other by disulfide bonds.

In some embodiments, the isolated IA protein complex according to the present disclosure comprises at least three antigens comprise different amino acid sequences.

In an advantageous embodiment, the isolated protein complex according to the present disclosure comprises at least four recombinant fusion proteins:
a) a first fusion protein unit comprises the immunoglobulin heavy chain constant domains C_{H}1 and C_{H}3 and a first antigen, wherein said first antigen is fused N-terminal to the C_{H}1-domain (HC unit 1); and
b) a second fusion protein unit comprises an immunoglobulin light chain constant domain C_{L}, and a second antigen, wherein said second antigen is fused N-terminal to the C_{L}-domain (LC unit 1), and wherein said first and said second fusion protein unit are covalently linked to each other, in particular by at least one disulfide bond,
c) a third fusion protein unit comprises the immunoglobulin heavy chain constant domains C_{H}1 and C_{H}3 and a third antigen, wherein said third antigen is fused N-terminal to the C_{H}1-domain of said third fusion protein unit (HC unit 2), wherein said HC unit 1 and HC unit 2 are covalently linked to each other, in particular by at least one disulfide bond,
d) a fourth fusion protein unit comprises an immunoglobulin light chain constant domain C_{L}, and a fourth antigen, wherein said fourth antigen is fused N-terminal to the C_{L}-domain of said fourth fusion protein (LC unit 2), and wherein said third and the fourth fusion protein unit are covalently linked to each other, in particular by at least one disulfide bond.

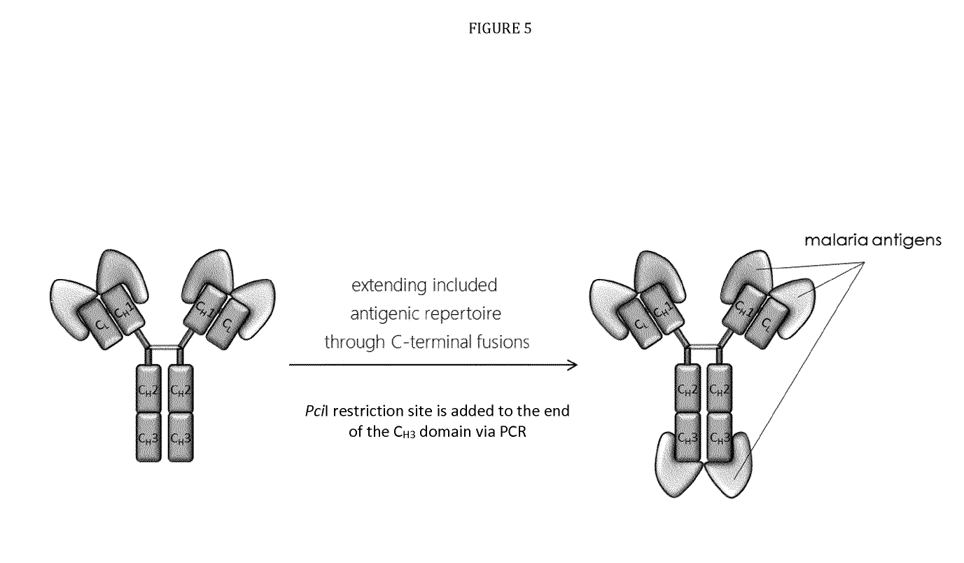

In some embodiments, the fusion proteins comprises further additional antigens, wherein said additional antigens are fused N-terminal and/or C-terminal to said recombinant fusion protein (see FIG. 5).

A) HC Fusion Polypeptide

As mentioned above, in some advantageous embodiments an isolated IA protein complexes of the present disclosure comprise at least two recombinant fusion proteins, wherein the first fusion protein comprises the immunoglobulin heavy chain constant domains C_{H}1 and C_{H}3 and a first antigen, wherein said first antigen is linked/fused N-terminal and/or C-terminal to the immunoglobulin heavy chain constant domains (HC fusion polypeptide unit or HC unit 1).

In further advantageous embodiments, the isolated protein complex according to the present disclosure comprises further a third recombinant fusion protein comprising the immunoglobulin heavy chain constant domains C_{H}1 and C_{H}3 and a third antigen, wherein said third antigen is fused N-terminal and/or C-terminal to the immunoglobulin heavy chain constant domains of said third fusion protein (HC unit 2).

In the following the first and the second HC fusion polypeptide units will be referred to as HC fusion polypeptide.

Typically, in the HC fusion polypeptides comprised in the isolated protein complex according to the present disclosure comprises an amino acid sequence of a IgG, IgM, or IgA heavy chain constant region; or variant thereof. In particular, each of the immunoglobulin heavy chain constant domains comprise an amino acid sequence of a mammalian heavy chain constant domain, preferably a human heavy chain constant domain; or variant thereof. Preferably, each of the immunoglobulin heavy chain constant domains comprise an amino acid sequence or a variant thereof of a IgG heavy chain constant domain, preferably a human IgG, preferably human IgG1 (see e.g. SEQ ID NO. 32).

The phrase “heavy chain,” or “immunoglobulin heavy chain” includes an immunoglobulin heavy chain constant region sequence from any organism. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a C_{H}1 domain, a hinge, a C_{H}2 domain, and a C_{H}3 domain (see Kapelski et al., Malaria Journal 2015, 14:50). According to the Kabat numbering scheme the positions may be for HC_hIgG1: EU/Kabat numbering: 121-447/117-478 and for hLCkappa:EU/Kabat numbering: 111-214. Examples for A functional fragment of a heavy chain includes a fragment that is capable of specifically recognizing an epitope (e.g., recognizing the epitope with a KD in the micromolar, nanomolar, or picomolar range), that is capable of expressing and secreting from a cell, and that comprises at least one CDR.

The term “immunoglobulin heavy chain constant domain” is a polypeptide derived from a native immunoglobulin heavy chain region, or variant or fragment thereof. Typically, the immunoglobulin heavy chain constant domains are part of the Fc receptor binding portion typically comprises the Fc portion of an immunoglobulin, or fragment or variant thereof.

The term “Fc portion” includes a fragment of an IgG molecule that is obtained by limited proteolysis with the enzyme papain, which acts on the hinge region of IgG. An Fc portion obtained in this way contains two identical disulfide linked peptides containing the heavy chain C_{H}2 and C_{H}3 domains of IgG, also referred to as Cy2 and Cy3 domains respectively. The two peptides may be linked by two disulfide bonds between cysteine residues in the N-terminal parts of the peptides. “Fc portion” also includes the corresponding portion of any of the other four immunoglobulin classes, namely IgM, IgA, IgD or IgE. The Fc portion of igM contains two identical disulphide linked peptide heavy chain C_{H}2, C_{H}3 and C_{H}4 domains, also referred to as Cp2, Cp3 and Cp4. The peptides may be disulphide linked at a cysteine residue occurring between the Cp2 and Cp3 domains. The Fc portion of IgA contains two identical disulphide linked peptide heavy chain C_{H}2 and C_{H}3 domains, also referred to as Ca2 and Ca3. The peptides are disulphide linked at a cysteine residue occurring N-terminal to the Cp2 domain. The arrangements of the disulphide linkages described for IgG, IgM and IgA pertain to natural human antibodies. There may be some variation among antibodies from other mammalian species, although such antibodies may be suitable in the context of the present invention. Antibodies are also found in birds, reptiles and amphibians, and they may likewise be suitable. Nucleotide and amino acid sequences of human Fc IgG are disclosed, for example, in Ellison et al. (1982) NUCLEIC ACIDS RES. 10: 4071-4079. Nucleotide and amino acid sequences of murine Fc lgG2a are disclosed, for example, in Bourgois et al. (1974) EUR. J. BIOCHEM. 43; 423-435.

In some embodiments, the HC fusion polypeptide comprises an Fc receptor binding portion. Examples for Fc receptor binding portions are SEQ ID NO. 32 SEQ ID NO: 76 and SEQ ID NO. 77, or variants thereof.

Typically, in a HC fusion polypeptide comprised in a protein complex according to the present disclosure, each of the immunoglobulin heavy chain constant regions comprises an amino acid sequence of a IgG, IgM, or IgA heavy chain constant region; or variant thereof. Typically, each of the immunoglobulin heavy chain constant regions comprises an amino acid sequence of a mammalian heavy chain constant region, preferably a human heavy chain constant region; or variant thereof. Suitably, each of the immunoglobulin heavy chain constant regions comprises an amino acid sequence of a IgG heavy chain constant region, preferably a human IgG. Suitable human IgG subtypes are IgG1, IgG2, IgG3 and lgG4, although igG1 or lgG3 are preferred.

According to the present disclosure, the C_{H}1 and C_{H}3 may be linked to each other in a HC fusion polypeptide according to the present disclosure. In the present disclosure the term “Linked” refers to non-covalent or covalent bonding between two or more molecules. Linking may be direct or indirect. Two molecules are indirectly linked when the two molecules are linked via a connecting molecule (linker), like a hinge region. Two molecules are directly linked when there is no intervening molecule linking them.

As mentioned above, the immunoglobulin heavy chain constant domains C_{H}1 and C_{H}3 in a HC fusion polypeptide according to the present disclosure may be linked either directly or indirectly to each other, preferably via peptide bonds or disulfide bonds. An example of indirect covalent linking is that an intervening amino acid sequence, such as a peptide linker is juxtaposed between segments forming the fusion protein.

In some embodiments, C_{H}1 and C_{H}3 are directly linked to each other. In other embodiments, the C_{H}1 and C_{H}3 are indirectly linked to each other via the human IGG1 hinge region which comprises fifteen amino acids (EU no: E₂₁₆-P₂₃₀; Kabat no: E₂₂₆-P₂₄₃), wherein in some examples the linker is a polypeptide with a size of less or equal twenty amino acids, in particular 2 to 6 amino acids. For example, the C_{H}1 and C_{H}3 may indirectly linked to each other by a hinge region of the immunoglobulin which occurs normally between C_{H}1 and C_{H}2 domains in a native immunoglobulin. Furthermore, in some advantageous embodiments, an immunoglobulin heavy chain constant domain C_{H}2 may be between the C_{H}1 and C_{H}3 in the first fusion protein. However, In some advantageous embodiments, the first fusion protein (HC unit 1) lacks a C_{H}2 domain and/or a heavy chain variable region domain (VH).

In other advantageous embodiments, a HC fusion polypeptide according to the present disclosure protein comprises also the immunoglobulin heavy chain constant domain C_{H}2. In these embodiments, the C_{H}1 and C_{H}2 are directly linked to each other or the C_{H}1 and C_{H}2 are indirectly linked to each other via a linker like a hinge region as described above. For example, the C_{H}1 and C_{H}2 may indirectly linked to each other by a hinge region of an immunoglobulin which occurs between C_{H}1 and C_{H}2 domains in a native immunoglobulin. Examples of such suitable hinge regions are shown in the IMGT Repertoire database of INTERNATIONAL IMMUNOGENETICS INFORMATION SYSTEM (http://www.imgt.org), a global reference in immunogenetics and immunoinformatics.

Therefore, in some embodiments, the HC fusion polypeptide comprises a hinge region. In particular, the HC fusion polypeptide comprises a hinge region between the C_{H}1-domain and the C_{H}3-domain. In other embodiments, the HC fusion polypeptide comprises a hinge region between the C_{H}1-domain and the C_{H}2-domain. In some other embodiments, the HC fusion polypeptides (HC units 1 and 2) comprise the immunoglobulin heavy chain constant domains C_{H}1, C_{H}2 and C_{H}3 and a hinge region between the C_{H}1-domain and the C_{H}2-domain.

As mentioned above, in advantageous embodiments of the present disclosure, the first fusion protein in the isolated protein complex comprises the immunoglobulin heavy chain constant domains C_{H}1 and C_{H}3 and a first antigen.

According to the present disclosure, a first antigen may be linked/fused N-terminal and/or C-terminal to the immunoglobulin heavy chain constant domains of the first fusion protein (HC fusion polypeptide). The antigen may be may be linked either directly or indirectly N-terminal to the C_{H}1 domain and/or C-terminal to the C_{H}3 domain of the first fusion protein. In some embodiments, a first antigen is linked N-terminal to the C_{H}1 domain and another antigen is linked C-terminal to the C_{H}3. The other antigen may derived from the same or from a different polypeptide, for example the antigen linked N-terminal to the C_{H}1 domain comprises the identical or at least 85% identical amino acid sequence as the antigen linked C-terminal to the C_{H}3 domain. In some advantageous embodiments, the antigen linked N-terminal to the C_{H}1 domain comprises a different amino acid sequence as the antigen linked C-terminal to the C_{H}3 domain. Therefore, a higher variability is given in the protein complex.

In another embodiment, a third antigen may be linked/fused N-terminal and/or C-terminal to the immunoglobulin heavy chain constant domains of the third fusion protein (HC unit 2). The antigen may be may be linked either directly or indirectly N-terminal to the C_{H}1 domain and/or C-terminal to the C_{H}3 domain of the first fusion protein. In some embodiments, a first antigen is linked N-terminal to the C_{H}1 domain and another antigen is linked C-terminal to the C_{H}3. The other antigen may derived from the same or from a different polypeptide, for example the antigen linked N-terminal to the C_{H}1 domain comprises the identical or at least 85% identical amino acid sequence as the antigen linked C-terminal to the C_{H}3 domain. In some advantageous embodiments, the antigen linked N-terminal to the C_{H}1 domain comprises a different amino acid sequence as the antigen linked C-terminal to the C_{H}3 domain. Therefore, a higher variability is given in the protein complex. In particular, the first antigen in said first fusion protein is fused N-terminally to the C_{H}1-domain and a further antigen in said first fusion protein is fused C-terminally to the C_{H}3-domain.

In some advantageous embodiments, a third antigen in said third fusion protein is fused N-terminally to the C_{H}1-domain and a further antigen in said third fusion protein (HC unit 2) is fused C-terminally to the C_{H}3-domain. In particular, the fused/linked antigens comprise different amino acid sequences

In the following table 2, examples of HC fusion polypeptides are shown.

TABLE 2
MIA HC fusions: Malaria Immunoassemblin heavy chain fusion polypeptides

| | | |
|---|---|---|
| 32 | human IgG1 | KGPSVFPLAPSSKSTSGGTAALGCLVKDYFPEPVTVSWNSGALTS |
| | constant domain | GVHTFPAVLQSSGLYSLSSVVTVPSSSLGTQTYICNVNHKPSNTKV |
| | (HC) | DKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYR |
| | | VVSVLTVLHQDWLNGKEYKCKVSNKAFPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHYT |
| | | QKSLSLSPGKSEKDEL |
| 33 | PfMSP1₁₉_3D7- | ISQHQCVKKQCPENSGCFRHLDEREECKCLLNYKQEGDKCVENP |
| | HC-ER | NPTCNENNGGCDADATCTEEDSGSSRKKITCECTKPDSYPLFDGI |
| | | FCSSSNAAAKGPSVFPLAPSSKSTSGGTAALGCLVKDYFPEPVTV |
| | | SWNSGALTSGVHTFPAVLQSSGLYSLSSVVTVPSSSLGTQTYICNV |
| | | NHKPSNTKVDKKVEPKSCDKTHTCPPCPAPELLGGPSVFLFPPKP |
| | | KDTLMISRTPEVTCVVVDVSHEDPEVKFNWYVDGVEVHNAKTKPR |
| | | EEQYNSTYRVVSVLTVLHQDWLNGKEYKCKVSNKAFPAPIEKTISK |
| | | AKGQPREPQVYTLPPSRDELTKNQVSLTCLVKGFYPSDIAVEWES |
| | | NGQPENNYKTTPPVLDSDGSFFLYSKLTVDKSRWQQGNVFSCSV |
| | | MHEALHNHYTQKSLSLSPGKSEKDEL |
| 34 | Tetra_MSP1₁₉-HC- | ISQHQCVKKQCPENSGCFRHLDEREECKCLLNYKQEGDKCVENP |
| | ER | NPTCNENNGGCDADATCTEEDSGSSRKKITCECTKPDSYPLFDGI |
| | (strain- | FCSSSNAAVAMAISQHQCVKKQCPENSGCFRHLDEREECKCLLNY |
| | transcending) | KQEGDKCVENPNPTCNENNGGCDADAKCTEEDSGSNGKKITCEC |
| | | TKPDSYPLFDGIFCSSSNAAVAMAISQHQCVKKQCPQNSGCFRHL |
| | | DEREECKCLLNYKQEGDKCVENPNPTCNENNGGCDADAKCTEED |
| | | SGSNGKKITCECTKPDSYPLFDGIFCSSSNAAVAMAISQHQCVKKQ |
| | | CPQNSGCFRHLDEREECKCLLNYKQEGDKCVENPNPTCNENNGG |
| | | CDADAKCTEEDSGSNGKKITCECTKPDSYPFFDGIFCSSSNAAAK |
| | | GPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRV |
| | | VSVLTVLHQDWLNGKEYKCKVSNKAFPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYT |
| | | QKSLSLSPGKSEKDEL |
| 35 | Tetra_MSP1₁₉-HC- | ISQHQCVKKQCPENSGCFRHLDEREECKCLLNYKQEGDKCVENP |
| | PciI-MSP1₁₉_3D7- | NPTCNENNGGCDADATCTEEDSGSSRKKITCECTKPDSYPLFDGI |
| | ERH | FCSSSNAAVAMAISQHQCVKKQCPENSGCFRHLDEREECKCLLNY |
| | aka | KQEGDKCVENPNPTCNENNGGCDADAKCTEEDSGSNGKKITCEC |
| | PciI vector (for MIA- | TKPDSYPLFDGIFCSSSNAAVAMAISQHQCVKKQCPQNSGCFRHL |
| | C cloning) | DEREECKCLLNYKQEGDKCVENPNPTCNENNGGCDADAKCTEED |
| | (strain- | SGSNGKKITCECTKPDSYPLFDGIFCSSSNAAVAMAISQHQCVKKQ |
| | transcending) | CPQNSGCFRHLDEREECKCLLNYKQEGDKCVENPNPTCNENNGG |
| | | CDADAKCTEEDSGSNGKKITCECTKPDSYPFFDGIFCSSSNAAAK |
| | | GPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRV |
| | | VSVLTVLHQDWLNGKEYKCKVSNKAFPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYT |
| | | QKSLSLSPGKHMLNISQHQCVKKQCPENSGCFRHLDEREECKCLL |
| | | NYKQEGDKCVENPNPTCNENNGGCDADATCTEEDSGSSRKKITC |
| | | ECTKPDSYPLFDGIFCSSSNAAAHHHHHHSEKDEL |
| 36 | Pfs25_FKO-HC-ER | VTVDTVCKRGFLIQMSGHLECKCENDLVLVNEETCEEKVLKCDEKT |
| | | VNKPCGDFSKCIKIDGNPVSYACKCNLGYDMVNNVCIPNECKNVA |
| | | CGNGKCILDTSNPVKTGVCSCNIGKVPNVQDQKCSKDGETKCSLK |
| | | CLKENEACKAVDGIYKCDCKDGFIIDNEASICTAAAKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKAFPAPIEKTISKAKGQPREPQVYTLPPSRDEL |
| | | TKNQVSLTCLVKGFYPSDIAVEWESNGQPENNYKTTPPVLDSDGS |
| | | FFLYSKLTVDKSRWQQGNVFSCSVMHEALHNHYTQKSLSLSPGKS |
| | | EKDEL |
| 37 | Pfs25_SHKO-HC- | VTVDTVCKRGFLIQMSGHLECKCENDTVLVNEETCEEKVLKCDEK |
| | ER | TVNKPCGDFSKCIKIDGNPVSYACKCNLGYDMVNNVCIPNECKNVT |
| | | CGNGKCILDTSNPVKTGVCSCNIGKVPNVQDQKCSKDGETKCSLK |
| | | CLKENETCKAVDGIYKCDCKDGFIIDNEASICTAAAKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSH |
| | | EDPEVKFNWYVDGVEVHNAKTKPREEQYNSTYRVVSVLTV |
| | | LHQDWLNGKEYKCKVSNKAFPAPIEKTISKAKGQPREPQVYTLPPS |
| | | RDELTKNQVSLTCLVKGFYPSDIAVEWESNGQPENNYKTTPPVLD |
| | | SDGSFFLYSKLTVDKSRWQQGNVFSCSVMHEALHNHYTQKSLSL |
| | | SPGKSEKDEL |
| 38 | Pfs28-HC-ER | VTENTICKYGYLIQMSNHYECKCIEGYVLINEDTCGKKVVCDKVEN |
| | | SFKACDEYAYCFDLGNKNNEKQIKCMCRTEYTLTAGVCVPNVCRD |
| | | KVCGKGKCIVDPANSLTHTCSCNIGTILNQNKLCDIQGDTPCSLKCA |
| | | ENEVCTLEGNYYTCKEDPSSNGGGNTVDQASAAAKGPSVFPLAP |
| | | SSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQ |
| | | DWLNGKEYKCKVSNKAFPAPIEKTISKAKGQPREPQVYTLPPSRDE |
| | | LTKNQVSLTCLVKGFYPSDIAVEWESNGQPENNYKTTPPVLDSDG |
| | | SFFLYSKLTVDKSRWQQGNVFSCSVMHEALHNHYTQKSLSLSPGK |
| | | SEKDEL |
| 39 | PfCSP_TSR-HC- | PSDKHIEQYLKKIQNSLSTEWSPCSVTCGNGIQVRIKPGSANKPKD |
| | ER | ELDYENDIEKKICKMEKCSSVFNVVNSSAAAKGPSVFPLAPSSKST |
| | | SGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTC |
| | | PPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNG |
| | | KEYKCKVSNKAFPAPIEKTISKAKGQPREPQVYTLPPSRDELTKNQ |
| | | VSLTCLVKGFYPSDIAVEWESNGQPENNYKTTPPVLDSDGSFFLYS |
| | | KLTVDKSRWQQGNVFSCSVMHEALHNHYTQKSLSLSPGKSEKDE |
| | | L |
| 40 | PfMTRAP_TSR- | THDTCDEWSEWSACTHGISTRKCLSDSSIKDETLVCTKCDKWGE |
| | HC-ER | WSECKDGRMHRKVLNCPFIKEEQECDVNNEAAAKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKAFPAPIEKTISKAKGQPREPQVYTLPPSRDEL |
| | | TKNQVSLTCLVKGFYPSDIAVEWESNGQPENNYKTTPPVLDSDGS |
| | | FFLYSKLTVDKSRWQQGNVFSCSVMHEALHNHYTQKSLSLSPGKS |
| | | EKDEL |
| 41 | PfCTMT-HC-ER | PSDKHIEQYLKKIQNSLSTEWSPCSVTCGNGIQVRIKPGSANKPKD |
| | (stage- | ELDYENDIEKKICKMEKCSSVFNVVNSSAAVAMAEKTASCGVWDE |
| | transcending) | WSPCSVTCGKGTRSRKREILHEGCTSELQEQCEEERCLPKAAVA |
| | | MATHDTCDEWSEWSACTHGISTRKCLSDSSIKDETLVCTKCDKWG |
| | | EWSECKDGRMHRKVLNCPFIKEEQECDVNNEAAVAMAFYSEWGE |
| | | WSNCAMDCDHPDNVQIRERECIHPSGDCFKGDLKESRPCIIPLPPC |
| | | NELFSHKDNSAFKAAAKGPSVFPLAPSSKSTSGGTAALGCLVKDY |
| | | FPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEV |
| | | HNAKTKPREEQYNSTYRVVSVLTVLHQDWLNGKEYKCKVSNKAFP |
| | | APIEKTISKAKGQPREPQVYTLPPSRDELTKNQVSLTCLVKGFYPS |
| | | DIAVEWESNGQPENNYKTTPPVLDSDGSFFLYSKLTVDKSRWQQ |
| | | GNVFSCSVMHEALHNHYTQKSLSLSPGKSEKDEL |
| 42 | PfMSP3A_Nterm- | SKEIVKKYNLNLRNAILNNNSQIENEENVNTTITGNDFSGGEFLWPG |
| | HC-ER | YTEELKAKKASEDAEKAANDAENASKEAEEAAKEAVNLKESDKSY |
| | | TKAKEAATAASKAKKAVETALKAKDDAEKSSKADSISTKTKEYAEK |
| | | AKNAYEKAKNAYQKANQAVLKAKEASSYDYILGWEFGGGVPEHKK |
| | | EENMLSHLYVSSKDKENIAKENDDVLDEKEEEAEETEEEELEEKNE |
| | | EETESEISEDEEEEEEEEKEEENDKKKEQEKEQSNENNDQKKDME |
| | | AQNLISKNQNNNEKNVKEAAESIMKTLAGLIKGNNQIDSTLKDLVEE |
| | | LSKYFKNHAAAKGPSVFPLAPSSKSTSGGTAALGCLVKDYFPEPVT |
| | | VSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPK |
| | | PKDTLMISRTPEVTCVVVDVSHEDPEVKFNWYVDGVEVHNAKTKP |
| | | REEQYNSTYRVVSVLTVLHQDWLNGKEYKCKVSNKAFPAPIEKTIS |
| | | KAKGQPREPQVYTLPPSRDELTKNQVSLTCLVKGFYPSDIAVEWE |
| | | SNGQPENNYKTTPPVLDSDGSFFLYSKLTVDKSRWQQGNVFSCS |
| | | VMHEALHNHYTQKSLSLSPGKSEKDEL |
| 43 | PfMSP3B_Nterm- | SKEIVKKYNLNLRNAILNNNSQIENEENDIKYELNEQNDENVNTPIV |
| | HC-ER | GNMEFGEGFSADDQKDIEAYKKAKQASQDAEQAAKDAENAAKDA |
| | | EEAAKDAEKLKESDESYTKAKEACTAASKAKKAVETALKAKDDAET |
| | | ALKTSETPEKPSRINLFSRKTKEYAEKAKNAYEKAKNAYQKANQAV |
| | | LKAKEASSYDYILGWEFGGGVPEHKKEENMLSHLYVSSKDKENIAK |
| | | ENDDVLDEKEEEAEETEEEELEEKNEEETESEISEDEEEEEEEEKE |
| | | EENDKKKEQEKEQSNENNDQKKDMEAQNLISKNQNNNEKNVKEA |
| | | AESIMKTLAGLIKGNNQIDSTLKDLVEELSKYFKNHAAAKGPSVFPL |
| | | APSSKSTSGGTAALGCLVKDYFPEPVTVSWNSGALTSGVHTFPAV |
| | | LQSSGLYSLSSVVTVPSSSLGTQTYICNVNHKPSNTKVDKKVEPKS |
| | | CDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLH |
| | | QDWLNGKEYKCKVSNKAFPAPIEKTISKAKGQPREPQVYTLPPSR |
| | | DELTKNQVSLTCLVKGFYPSDIAVEWESNGQPENNYKTTPPVLDS |
| | | DGSFFLYSKLTVDKSRWQQGNVFSCSVMHEALHNHYTQKSLSLS |
| | | PGKSEKDEL |
| 44 | PfMSP6₃₆-HC-ER | NGLTGATENIAQVVQANSETNKNPTSHSNSTTTSLNNNILGWEFG |
| | | GGAPQNGAAEDKKTEYLLEQIKIPSWDRNNIPDENEQVIEDPQEDN |
| | | KDEDEDEETETENLETEDDNNEEIEENEEDDIDEESVEEKEEEEEK |
| | | KEEEEKKEEKKEEKKPDNEITNEVKEEQKYSSPSDINAQNLISNKN |
| | | KKNDETKKTAENIVKTLVGLFNEKNEIDSTINNLVQEMIHLFSSKSR |
| | | WSAAAKGPSVFPLAPSSKSTSGGTAALGCLVKDYFPEPVTVSWNS |
| | | GALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTL |
| | | MISRTPEVTCVVVDVSHEDPEVKFNWYVDGVEVHNAKTKPREEQY |
| | | NSTYRVVSVLTVLHQDWLNGKEYKCKVSNKAFPAPIEKTISKAKGQ |
| | | PREPQVYTLPPSRDELTKNQVSLTCLVKGFYPSDIAVEWESNGQP |
| | | ENNYKTTPPVLDSDGSFFLYSKLTVDKSRWQQGNVFSCSVMHEAL |
| | | HNHYTQKSLSLSPGKSEKDEL |
| 45 | PfMSP7₂₂-HC-ER | SETDTQSKNEQEISTQGQEVQKPAQGGESTFQKDLDKKLYNLGDV |
| | | FNHVVDISNKENKINLDEHDKKYTDFKKEYEDFVLNSKEYDIIKNLII |
| | | MFGQEDNKAKNGKTDIVSEAKHITEIFIKLFKDKEYHEQFKNYIYGV |
| | | YSYAKQNSHLSEKKIKQEEEYKKFLEYSFNLLNTAAAKGPSVFPLA |
| | | PSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLH |
| | | QDWLNGKEYKCKVSNKAFPAPIEKTISKAKGQPREPQVYTLPPSR |
| | | DELTKNQVSLTCLVKGFYPSDIAVEWESNGQPENNYKTTPPVLDS |
| | | DGSFFLYSKLTVDKSRWQQGNVFSCSVMHEALHNHYTQKSLSLS |
| | | PGKSEKDEL |
| 46 | PfExp1-HC-ER | EKTNKGTGSGVSSKKKNKKGSGEPLIDVHDLISDMIKKEEELVEVN |
| | | KRKSKYKLATSNTEKGRHPFKIGSSDPADNANPDADSESNGEPNA |
| | | DPQVTAQDVTPEQPQGDDNNLVSGPEHAAAKGPSVFPLAPSSKS |
| | | TSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHT |
| | | CPPCPAPELLGGPSVFLFPPKPKDTLMISRTPEVTCVVVDVSHEDP |
| | | EVKFNWYVDGVEVHNAKTKPREEQYNSTYRVVSVLTVLHQDWLN |
| | | GKEYKCKVSNKAFPAPIEKTISKAKGQPREPQVYTLPPSRDELT |
| | | KNQVSLTCLVKGFYPSDIAVEWESNGQPENNYKTTPPVLDSDGSF |
| | | FLYSKLTVDKSRWQQGNVFSCSVMHEALHNHYTQKSLSLSPGKS |
| | | EKDEL |
| 47 | PfEBA175_F2-HC- | DKNSVDTNTKVWECKKPYKLSTKDVCVPPRRQELCLGNIDRIYDK |
| | ER | NLLMIKEHILAIAIYESRILKRKYKNKDDKEVCKIINKAFADIRDIIGGT |
| | | DYWNDLSNRKLVGKINTNSNYVHRNKQNDKLFRDEWWKVIKKDV |
| | | WNVISWVFKDKTVCKEDDIENIPQFFRWFSEWGDDYCQDKTKMIE |
| | | TLKVECKEKPCEDDNCKRKCNSYKEWISKKKEEYNKQAKQYQEY |
| | | QKGNNYKMYSEFKSIKPEVYLKKYSEKCSNLNFEDEFKEELHSDYK |
| | | NKCTMCPEVAAAKGPSVFPLAPSSKSTSGGTAALGCLVKDYFPEP |
| | | VTVSWNSGALTSGVHTFPAVLQSSGLYSLSSVVTVPSSSLGTQTYI |
| | | CNVNHKPSNTKVDKKVEPKSCDKTHTCPPCPAPELLGGPSVFLFP |
| | | PKPKDTLMISRTPEVTCVVVDVSHEDPEVKFNWYVDGVEVHNAKT |
| | | KPREEQYNSTYRVVSVLTVLHQDWLNGKEYKCKVSNKAFPAPIEK |
| | | TISKAKGQPREPQVYTLPPSRDELTKNQVSLTCLVKGFYPSDIAVE |
| | | WESNGQPENNYKTTPPVLDSDGSFFLYSKLTVDKSRWQQGNVFS |
| | | CSVMHEALHNHYTQKSLSLSPGKSEKDEL |
| 48 | PfAMA1_GKO-HC- | IEIVERSNYMGNPWTEYMAKYDIEEVHGSGIRVDLGEDAEVAGTQY |
| | ER | RLPSGKCPVFGKGIIIENSNTAFLTPVATGNQYLKDGGFAFPPTEPL |
| | | MSPMTLDEMRHFYKDNKYVKNLDELTLCSRHAGNMIPDNDKNSN |
| | | YKYPAVYDDKDKKCHILYIAAQENNGPRYCNKDESKRNSMFCFRP |
| | | AKDISFQNYAYLSKNVVDNWEKVCPRKNLQNAKFGLWVDGNCEDI |
| | | PHVNEFPAIDLFECNKLVFELSASDQPKQYEQHLTDYEKIKEGFKN |
| | | KNAAMIKSAFLPTGAFKADRYKSHGKGYNWGNYNTETQKCEIFNV |
| | | KPTCLINNAAYIATTALSHPIEVENNFPCSLYKDEIMKEIERESKRIKL |
| | | NDNDDEGNKKIIAPRIFISDDKDSLKCPCDPEMVSNSACRFFVCKC |
| | | VERRAEVTSNNEVVVKEEYKDEYADIPEHKPTYDKMKAAAKGPSV |
| | | FPLAPSSKSTSGGTAALGCLVKDYFPEPVTVSWNSGALTSGVHTF |
| | | PAVLQSSGLYSLSSVVTVPSSSLGTQTYICNVNHKPSNTKVDKKVE |
| | | PKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVL |
| | | TVLHQDWLNGKEYKCKVSNKAFPAPIEKTISKAKGQPREPQVYTLP |
| | | PSRDELTKNQVSLTCLVKGFYPSDIAVEWESNGQPENNYKTTPPV |
| | | LDSDGSFFLYSKLTVDKSRWQQGNVFSCSVMHEALHNHYTQKSL |
| | | SLSPGKSEKDEL |
| 49 | PfRON2L-HC-ER | MDITQQAKDIGAGPVASCFTTRMSPPQQICLNSVVNTALSTSTQSA |
| | | MKAAAKGPSVFPLAPSSKSTSGGTAALGCLVKDYFPEPVTVSWNS |
| | | GALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTL |
| | | MISRTPEVTCVVVDVSHEDPEVKFNWYVDGVEVHNAKTKPREEQY |
| | | NSTYRVVSVLTVLHQDWLNGKEYKCKVSNKAFPAPIEKTISKAKGQ |
| | | PREPQVYTLPPSRDELTKNQVSLTCLVKGFYPSDIAVEWESNGQP |
| | | ENNYKTTPPVLDSDGSFFLYSKLTVDKSRWQQGNVFSCSVMHEAL |
| | | HNHYTQKSLSLSPGKSEKDEL |

In the following table 3, examples of HC fusion polypeptides comprising an additional C-terminal fusion polypeptide are shown.

TABLE 3
MIA-C HC fusions: MIAs comprising an additional
C-terminal fusion polypeptide

| | | |
|---|---|---|
| 67 | Tetra_MSP1₁₉-HC- | ISQHQCVKKQCPENSGCFRHLDEREECKCLLNYKQEGDKCVENP |
| | Pfs28-ERH | NPTCNENNGGCDADATCTEEDSGSSRKKITCECTKPDSYPLFDGI |
| | (stage and strain- | FCSSSNAAVAMAISQHQCVKKQCPENSGCFRHLDEREECKCLLNY |
| | transcending) | KQEGDKCVENPNPTCNENNGGCDADAKCTEEDSGSNGKKITCEC |
| | | TKPDSYPLFDGIFCSSSNAAVAMAISQHQCVKKQCPQNSGCFRHL |
| | | DEREECKCLLNYKQEGDKCVENPNPTCNENNGGCDADAKCTEED |
| | | SGSNGKKITCECTKPDSYPLFDGIFCSSSNAAVAMAISQHQCVKKQ |
| | | CPQNSGCFRHLDEREECKCLLNYKQEGDKCVENPNPTCNENNGG |
| | | CDADAKCTEEDSGSNGKKITCECTKPDSYPFFDGIFCSSSNAAAK |
| | | GPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRV |
| | | VSVLTVLHQDWLNGKEYKCKVSNKAFPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYT |
| | | QKSLSLSPGKHMVTENTICKYGYLIQMSNHYECKCIEGYVLINEDTC |
| | | GKKVVCDKVENSFKACDEYAYCFDLGNKNNEKQIKCMCRTEYTLT |
| | | AGVCVPNVCRDKVCGKGKCIVDPANSLTHTCSCNIGTILNQNKLCD |
| | | IQGDTPCSLKCAENEVCTLEGNYYTCKEDPSSNGGGNTVDQASAA |
| | | AHHHHHHSEKDEL |
| 68 | Tetra_MSP1₁₉-HC- | ISQHQCVKKQCPENSGCFRHLDEREECKCLLNYKQEGDKCVENP |
| | PfCSP_TSR-ERH | NPTCNENNGGCDADATCTEEDSGSSRKKITCECTKPDSYPLFDGI |
| | (stage and strain- | FCSSSNAAVAMAISQHQCVKKQCPENSGCFRHLDEREECKCLLNY |
| | transcending) | KQEGDKCVENPNPTCNENNGGCDADAKCTEEDSGSNGKKITCEC |
| | | TKPDSYPLFDGIFCSSSNAAVAMAISQHQCVKKQCPQNSGCFRHL |
| | | DEREECKCLLNYKQEGDKCVENPNPTCNENNGGCDADAKCTEED |
| | | SGSNGKKITCECTKPDSYPLFDGIFCSSSNAAVAMAISQHQCVKKQ |
| | | CPQNSGCFRHLDEREECKCLLNYKQEGDKCVENPNPTCNENNGG |
| | | CDADAKCTEEDSGSNGKKITCECTKPDSYPFFDGIFCSSSNAAAK |
| | | GPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRV |
| | | VSVLTVLHQDWLNGKEYKCKVSNKAFPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYT |
| | | QKSLSLSPGKHMAPSDKHIEQYLKKIQNSLSTEWSPCSVTCGNGIQ |
| | | VRIKPGSANKPKDELDYENDIEKKICKMEKCSSVFNVVNSSAAAHH |
| | | HHHHSEKDEL |
| 69 | Tetra_MSP1₁₉-HC- | ISQHQCVKKQCPENSGCFRHLDEREECKCLLNYKQEGDKCVENP |
| | PfMSP3A-ERH | NPTCNENNGGCDADATCTEEDSGSSRKKITCECTKPDSYPLFDGI |
| | (strain- | FCSSSNAAVAMAISQHQCVKKQCPENSGCFRHLDEREECKCLLNY |
| | transcending) | KQEGDKCVENPNPTCNENNGGCDADAKCTEEDSGSNGKKITCEC |
| | | TKPDSYPLFDGIFCSSSNAAVAMAISQHQCVKKQCPQNSGCFRHL |
| | | DEREECKCLLNYKQEGDKCVENPNPTCNENNGGCDADAKCTEED |
| | | SGSNGKKITCECTKPDSYPLFDGIFCSSSNAAVAMAISQHQCVKKQ |
| | | CPQNSGCFRHLDEREECKCLLNYKQEGDKCVENPNPTCNENNGG |
| | | CDADAKCTEEDSGSNGKKITCECTKPDSYPFFDGIFCSSSNAAAK |
| | | GPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRV |
| | | VSVLTVLHQDWLNGKEYKCKVSNKAFPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYT |
| | | QKSLSLSPGKHMVAAGDLRISSKEIVKKYNLNLRNAILNNNSQIENE |
| | | ENVNTTITGNDFSGGEFLWPGYTEELKAKKASEDAEKAANDAENA |
| | | SKEAEEAAKEAVNLKESDKSYTKAKEAATAASKAKKAVETALKAKD |
| | | DAEKSSKADSISTKTKEYAEKAKNAYEKAKNAYQKANQAVLKAKEA |
| | | SSYDYILGWEFGGGVPEHKKEENMLSHLYVSSKDKENIAKENDDV |
| | | LDEKEEEAEETEEEELEEKNEEETESEISEDEEEEEEEEKEEENDK |
| | | KKEQEKEQSNENNDQKKDMEAQNLISKNQNNNEKNVKEAAESIMK |
| | | TLAGLIKGNNQIDSTLKDLVEELSKYFKNHSKSRWSAAAHHHHHHS |
| | | EKDEL |
| 70 | Tetra_MSP1₁₉-HC- | MAISQHQCVKKQCPENSGCFRHLDEREECKCLLNYKQEGDKCVE |
| | PfMSP3B-ERH | NPNPTCNENNGGCDADATCTEEDSGSSRKKITCECTKPDSYPLFD |
| | (strain- | GIFCSSSNAAVAMAISQHQCVKKQCPENSGCFRHLDEREECKCLL |
| | transcending) | NYKQEGDKCVENPNPTCNENNGGCDADAKCTEEDSGSNGKKITC |
| | | ECTKPDSYPLFDGIFCSSSNAAVAMAISQHQCVKKQCPQNSGCFR |
| | | HLDEREECKCLLNYKQE |
| | | GDKCVENPNPTCNENNGGCDADAKCTEEDSGSNGKKITCECTKP |
| | | DSYPLFDGIFCSSSNAAVAMAISQHQCVKKQCPQNSGCFRHLDER |
| | | EECKCLLNYKQEGDKCVENPNPTCNENNGGCDADAKCTEEDSGS |
| | | NGKKITCECTKPDSYPFFDGIFCSSSNAAAKGPSVFPLAPSSKSTS |
| | | GGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEV |
| | | KFNWYVDGVEVHNAKTKPREEQYNSTYRVVSVLTVLHQDWLNGK |
| | | EYKCKVSNKAFPAPIEKTISKAKGQPREPQVYTLPPSRDELTKNQV |
| | | SLTCLVKGFYPSDIAVEWESNGQPENNYKTTPPVLDSDGSFFLYSK |
| | | LTVDKSRWQQGNVFSCSVMHEALHNHYTQKSLSLSPGKHMVAAG |
| | | DLRISSKEIVKKYNLNLRNAILNNNSQIENEENDIKYELNEQNDENV |
| | | NTPIVGNMEFGEGFSADDQKDIEAYKKAKQASQDAEQAAKDAENA |
| | | AKDAEEAAKDAEKLKESDESYTKAKEACTAASKAKKAVETALKAKD |
| | | DAETALKTSETPEKPSRINLFSRKTKEYAEKAKNAYEKAKNAYQKA |
| | | NQAVLKAKEASSYDYILGWEFGGGVPEHKKEENMLSHLYVSSKDK |
| | | ENIAKENDDVLDEKEEEAEETEEEELEEKNEEETESEISEDEEEEEE |
| | | EEKEEENDKKKEQEKEQSNENNDQKKDMEAQNLISKNQNNNEKN |
| | | VKEAAESIMKTLAGLIKGNNQIDSTLKDLVEELSKYFKNHSKSRWSA |
| | | AAHHHHHHSEKDEL |
| 71 | Tetra_MSP1₁₉-HC- | ISQHQCVKKQCPENSGCFRHLDEREECKCLLNYKQEGDKCVENP |
| | PfMSP636-ERH | NPTCNENNGGCDADATCTEEDSGSSRKKITCECTKPDSYPLFDGI |
| | (strain- | FCSSSNAAVAMAISQHQCVKKQCPENSGCFRHLDEREECKCLLNY |
| | transcending) | KQEGDKCVENPNPTCNENNGGCDADAKCTEEDSGSNGKKITCEC |
| | | TKPDSYPLFDGIFCSSSNAAVAMAISQHQCVKKQCPQNSGCFRHL |
| | | DEREECKCLLNYKQEGDKCVENPNPTCNENNGGCDADAKCTEED |
| | | SGSNGKKITCECTKPDSYPLFDGIFCSSSNAAVAMAISQHQCVKKQ |
| | | CPQNSGCFRHLDEREECKCLLNYKQEGDKCVENPNPTCNENNGG |
| | | CDADAKCTEEDSGSNGKKITCECTKPDSYPFFDGIFCSSSNAAAK |
| | | GPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRV |
| | | VSVLTVLHQDWLNGKEYKCKVSNKAFPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYT |
| | | QKSLSLSPGKHMVAAGDLRISNGLTGATENIAQVVQANSETNKNPT |
| | | SHSNSTTTSLNNNILGWEFGGGAPQNGAAEDKKTEYLLEQIKIPSW |
| | | DRNNIPDENEQVIEDPQEDNKDEDEDEETETENLETEDDNNEEIEE |
| | | NEEDDIDEESVEEKEEEEEKKEEEEKKEEKKEEKKPDNEITNEVKE |
| | | EQKYSSPSDINAQNLISNKNKKNDETKKTAENIVKTLVGLFNEKNEI |
| | | DSTINNLVQEMIHLFSSKSRWSAAAHHHHHHSEKDEL |
| 72 | Tetra_MSP1₁₉-HC- | ISQHQCVKKQCPENSGCFRHLDEREECKCLLNYKQEGDKCVENP |
| | PfMSP722-ERH | NPTCNENNGGCDADATCTEEDSGSSRKKITCECTKPDSYPLFDGI |
| | (strain- | FCSSSNAAVAMAISQHQCVKKQCPENSGCFRHLDEREECKCLLNY |
| | transcending) | KQEGDKCVENPNPTCNENNGGCDADAKCTEEDSGSNGKKITCEC |
| | | TKPDSYPLFDGIFCSSSNAAVAMAISQHQCVKKQCPQNSGCFRHL |
| | | DEREECKCLLNYKQEGDKCVENPNPTCNENNGGCDADAKCTEED |
| | | SGSNGKKITCECTKPDSYPLFDGIFCSSSNAAVAMAISQHQCVKKQ |
| | | CPQNSGCFRHLDEREECKCLLNYKQEGDKCVENPNPTCNENNGG |
| | | CDADAKCTEEDSGSNGKKITCECTKPDSYPFFDGIFCSSSNAAAK |
| | | GPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRV |
| | | VSVLTVLHQDWLNGKEYKCKVSNKAFPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYT |
| | | QKSLSLSPGKHMGSETDTQSKNEQEISTQGQEVQKPAQGGESTF |
| | | QKDLDKKLYNLGDVFNHVVDISNKENKINLDEHDKKYTDFKKEYED |
| | | FVLNSKEYDIIKNLIIMFGQEDNKAKNGKTDIVSEAKHITEIFIKLFKDK |
| | | EYHEQFKNYIYGVYSYAKQNSHLSEKKIKQEEEYKKFLEYSFNLLN |
| | | TAAAHHHHHHSEKDEL |
| 73 | PfMTRAP_TSR- | THDTCDEWSEWSACTHGISTRKCLSDSSIKDETLVCTKCDKWGE |
| | HC-PfExp1-ERH | WSECKDGRMHRKVLNCPFIKEEQECDVNNEAAAKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKAFPAPIEKTISKAKGQPREPQVYTLPPSRDEL |
| | | TKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| | | HMAEKTNKGTGSGVSSKKKNKKGSGEPLIDVHDLISDMIKKEEELV |
| | | EVNKRKSKYKLATSNTEKGRHPFKIGSSDPADNANPDADSESNGE |
| | | PNADPQVTAQDVTPEQPQGDDNNLVSGPEHAAAHHHHHHSEKDEL |
| 74 | Tetra_MSP1₁₉-HC- | ISQHQCVKKQCPENSGCFRHLDEREECKCLLNYKQEGDKCVENP |
| | PfEBA175_F2-ERH | NPTCNENNGGCDADATCTEEDSGSSRKKITCECTKPDSYPLFDGI |
| | (strain- | FCSSSNAAVAMAISQHQCVKKQCPENSGCFRHLDEREECKCLLNY |
| | transcending) | KQEGDKCVENPNPTCNENNGGCDADAKCTEEDSGSNGKKITCEC |
| | | TKPDSYPLFDGIFCSSSNAAVAMAISQHQCVKKQCPQNSGCFRHL |
| | | DEREECKCLLNYKQEGDKCVENPNPTCNENNGGCDADAKCTEED |
| | | SGSNGKKITCECTKPDSYPLFDGIFCSSSNAAVAMAISQHQCVKKQ |
| | | CPQNSGCFRHLDEREECKCLLNYKQEGDKCVENPNPTCNENNGG |
| | | CDADAKCTEEDSGSNGKKITCECTKPDSYPFFDGIFCSSSNAAAK |
| | | GPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRV |
| | | VSVLTVLHQDWLNGKEYKCKVSNKAFPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYT |
| | | QKSLSLSPGKHMADKNSVDTNTKVWECKKPYKLSTKDVCVPPRR |
| | | QELCLGNIDRIYDKNLLMIKEHILAIAIYESRILKRKYKNKDDKEVCKII |
| | | NKAFADIRDIIGGTDYWNDLSNRKLVGKINTNSNYVHRNKQNDKLF |
| | | RDEWWKVIKKDVWNVISWVFKDKTVCKEDDIENIPQFFRWFSEWG |
| | | DDYCQDKTKMIETLKVECKEKPCEDDNCKRKCNSYKEWISKKKEE |
| | | YNKQAKQYQEYQKGNNYKMYSEFKSIKPEVYLKKYSEKCSNLNFE |
| | | DEFKEELHSDYKNKCTMCPEVAAAHHHHHHSEKDEL |
| 75 | Tetra_MSP1₁₉-HC- | ISQHQCVKKQCPENSGCFRHLDEREECKCLLNYKQEGDKCVENP |
| | PfRON2L-ERH | NPTCNENNGGCDADATCTEEDSGSSRKKITCECTKPDSYPLFDGI |
| | (strain- | FCSSSNAAVAMAISQHQCVKKQCPENSGCFRHLDEREECKCLLNY |
| | transcending) | KQEGDKCVENPNPTCNENNGGCDADAKCTEEDSGSNGKKITCEC |
| | | TKPDSYPLFDGIFCSSSNAAVAMAISQHQCVKKQCPQNSGCFRHL |
| | | DEREECKCLLNYKQEGDKCVENPNPTCNENNGGCDADAKCTEED |
| | | SGSNGKKITCECTKPDSYPLFDGIFCSSSNAAVAMAISQHQCVKKQ |
| | | CPQNSGCFRHLDEREECKCLLNYKQEGDKCVENPNPTCNENNGG |
| | | CDADAKCTEEDSGSNGKKITCECTKPDSYPFFDGIFCSSSNAAAK |
| | | GPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRV |
| | | VSVLTVLHQDWLNGKEYKCKVSNKAFPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYT |
| | | QKSLSLSPGKHMDITQQAKDIGAGPVASCFTTRMSPPQQICLNSVV |
| | | NTALSTSTQSAMKAAAHHHHHHSEKDEL |

In the following table 4 examples of HC fusion polypeptides with modified C_{H}3 regions for Fc-heterodimerization are shown.

TABLE 4
modMIA HC fusions: MIAs with modified
CH3 regions for Fc-heterodimerization

| | | |
|---|---|---|
| 76 | human IgG1 | KGPSVFPLAPSSKSTSGGTAALGCLVKDYFPEPVTVSWNSGALTS |
| | constant | GVHTFPAVLQSSGLYSLSSVVTVPSSSLGTQTYICNVNHKPSNTKV |
| | domain_E356K + | DKKVEPKSCDKTHTCPPCPAPELLGGPSVFLFPPKPKDTLMISRTP |
| | D399K (HC1.2, | EVTCVVVDVSHEDPEVKFNWYVDGVEVHNAKTKPREEQYNSTYR |
| | mutations are | VVSVLTVLHQDWLNGKEYKCKVSNKAFPAPIEKTISKAKGQPREPQ |
| | shown in bold) | VYTLPPSRDKLTKNQVSLTCLVKGFYPSDIAVWESNGQPENNYKT |
| | | TPPVLKSDGSFFLYSKLTVDKSRWQQGNVFSCSVMHEALHNHYT |
| | | QKSLSLSPGKSEKDEL |
| 77 | human IgG1 | KGPSVFPLAPSSKSTSGGTAALGCLVKDYFPEPVTVSWNSGALTS |
| | constant | GVHTFPAVLQSSGLYSLSSVVTVPSSSLGTQTYICNVNHKPSNTKV |
| | domain_K392D + | DKKVEPKSCDKTHTCPPCPAPELLGGPSVFLFPPKPKDTLMISRTP |
| | K409D (HC2.2, | EVTCVVVDVSHEDPEVKFNWYVDGVEVHNAKTKPREEQYNSTYR |
| | mutations are | VVSVLTVLHQDWLNGKEYKCKVSNKAFPAPIEKTISKAKGQPREPQ |
| | shown in bold) | VYTLPPSRDELTKNQVSLTCLVKGFYPSDIAVWESNGQPENNYDT |
| | | TPPVLDSDGSFFLYSDLTVDKSRWQQGNVFSCSVMHEALHNHYT |
| | | QKSLSLSPGKSEKDEL |
| 78 | PfMSP1₁₉_3D7- | ISQHQCVKKQCPENSGCFRHLDEREECKCLLNYKQEGDKCVENP |
| | HC1.2-ER | NPTCNENNGGCDADATCTEEDSGSSRKKITCECTKPDSYPLFDGI |
| | | FCSSSNAAAKGPSVFPLAPSSKSTSGGTAALGCLVKDYFPEPVTV |
| | | SWNSGALTSGVHTFPAVLQSSGLYSLSSVVTVPSSSLGTQTYICNV |
| | | NHKPSNTKVDKKVEPKSCDKTHTCPPCPAPELLGGPSVFLFPPKP |
| | | KDTLMISRTPEVTCVVVDVSHEDPEVKFNWYVDGVEVHNAKTKPR |
| | | EEQYNSTYRVVSVLTVLHQDWLNGKEYKCKVSNKAFPAPIEKTISK |
| | | AKGQPREPQVYTLPPSRDKLTKNQVSLTCLVKGFYPSDIAVEWES |
| | | NGQPENNYKTTPPVLKSDGSFFLYSKLTVDKSRWQQGNVFSCSV |
| | | MHEALHNHYTQKSLSLSPGKSEKDEL |
| 79 | PfMSP1₁₉_3D7- | ISQHQCVKKQCPENSGCFRHLDEREECKCLLNYKQEGDKCVENP |
| | HC2.2-ER | NPTCNENNGGCDADATCTEEDSGSSRKKITCECTKPDSYPLFDGI |
| | | FCSSSNAAAKGPSVFPLAPSSKSTSGGTAALGCLVKDYFPEPVTV |
| | | SWNSGALTSGVHTFPAVLQSSGLYSLSSVVTVPSSSLGTQTYICNV |
| | | NHKPSNTKVDKKVEPKSCDKTHTCPPCPAPELLGGPSVFLFPPKP |
| | | KDTLMISRTPEVTCVVVDVSHEDPEVKFNWYVDGVEVHNAKTKPR |
| | | EEQYNSTYRVVSVLTVLHQDWLNGKEYKCKVSNKAFPAPIEKTISK |
| | | AKGQPREPQVYTLPPSRDELTKNQVSLTCLVKGFYPSDIAVEWES |
| | | NGQPENNYDTTPPVLDSDGSFFLYSDLTVDKSRWQQGNVFSCSV |
| | | MHEALHNHYTQKSLSLSPGKSEKDEL |
| 80 | Pfs25_SHKO- | VTVDTVCKRGFLIQMSGHLECKCENDTVLVNEETCEEKVLKCDEK |
| | HC1.2-ER | TVNKPCGDFSKCIKIDGNPVSYACKCNLGYDMVNNVCIPNECKNVT |
| | | CGNGKCILDTSNPVKTGVCSCNIGKVPNVQDQKCSKDGETKCSLK |
| | | CLKENETCKAVDGIYKCDCKDGFIIDNEASICTAAAKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSH |
| | | EDPEVKFNWYVDGVEVHNAKTKPREEQYNSTYRVVSVLTV |
| | | LHQDWLNGKEYKCKVSNKAFPAPIEKTISKAKGQPREPQVYTLPPS |
| | | RDKLTKNQVSLTCLVKGFYPSDIAVEWESNGQPENNYKTTPPVLK |
| | | SDGSFFLYSKLTVDKSRWQQGNVFSCSVMHEALHNHYTQKSLSL |
| | | SPGKSEKDEL |
| 81 | Pfs25_SHKO- | VTVDTVCKRGFLIQMSGHLECKCENDTVLVNEETCEEKVLKCDEK |
| | HC2.2-ER | TVNKPCGDFSKCIKIDGNPVSYACKCNLGYDMVNNVCIPNECKNVT |
| | | CGNGKCILDTSNPVKTGVCSCNIGKVPNVQDQKCSKDGETKCSLK |
| | | CLKENETCKAVDGIYKCDCKDGFIIDNEASICTAAAKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKAFPAPIEKTISKAKGQPREPQVYTLPPSRDEL |
| | | TKNQVSLTCLVKGFYPSDIAVEWESNGQPENNYDTTPPVLDSDGS |
| | | FFLYSDLTVDKSRWQQGNVFSCSVMHEALHNHYTQKSLSLSPGK |
| | | SEKDEL |
| 82 | PfMTRAP_TSR- | THDTCDEWSEWSACTHGISTRKCLSDSSIKDETLVCTKCDKWGE |
| | HC1.2-ER | WSECKDGRMHRKVLNCPFIKEEQECDVNNEAAAKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKAFPAPIEKTISKAKGQPREPQVYTLPPSRDKL |
| | | TKNQVSLTCLVKGFYPSDIAVEWESNGQPENNYKTTPPVLKSDGS |
| | | FFLYSKLTVDKSRWQQGNVFSCSVMHEALHNHYTQKSLSLSPGKS |
| | | EKDEL |
| 83 | PfMTRAP_TSR- | THDTCDEWSEWSACTHGISTRKCLSDSSIKDETLVCTKCDKWGE |
| | HC2.2-ER | WSECKDGRMHRKVLNCPFIKEEQECDVNNEAAAKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKAFPAPIEKTISKAKGQPREPQVYTLPPSRDEL |
| | | TKNQVSLTCLVKGFYPSDIAVEWESNGQPENNYDTTPPVLDSDGS |
| | | FFLYSDLTVDKSRWQQGNVFSCSVMHEALHNHYTQKSLSLSPGK |
| | | SEKDEL |
| 84 | PfAMA1_GKO- | IEIVERSNYMGNPWTEYMAKYDIEEVHGSGIRVDLGEDAEVAGTQY |
| | HC1.2-ER | RLPSGKCPVFGKGIIIENSNTAFLTPVATGNQYLKDGGFAFPPTEPL |
| | | MSPMTLDEMRHFYKDNKYVKNLDELTLCSRHAGNMIPDNDKNSN |
| | | YKYPAVYDDKDKKCHILYIAAQENNGPRYCNKDESKRNSMFCFRP |
| | | AKDISFQNYAYLSKNVVDNWEKVCPRKNLQNAKFGLWVDGNCEDI |
| | | PHVNEFPAIDLFECNKLVFELSASDQPKQYEQHLTDYEKIKEGFKN |
| | | KNAAMIKSAFLPTGAFKADRYKSHGKGYNWGNYNTETQKCEIFNV |
| | | KPTCLINNAAYIATTALSHPIEVENNFPCSLYKDEIMKEIERESKRIKL |
| | | NDNDDEGNKKIIAPRIFISDDKDSLKCPCDPEMVSNSACRFFVCKC |
| | | VERRAEVTSNNEVVVKEEYKDEYADIPEHKPTYDKMKAAAKGPSV |
| | | FPLAPSSKSTSGGTAALGCLVKDYFPEPVTVSWNSGALTSGVHTF |
| | | PAVLQSSGLYSLSSVVTVPSSSLGTQTYICNVNHKPSNTKVDKKVE |
| | | PKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVL |
| | | TVLHQDWLNGKEYKCKVSNKAFPAPIEKTISKAKGQPREPQVYTLP |
| | | PSRDKLTKNQVSLTCLVKGFYPSDIAVEWESNGQPENNYKTTPPV |
| | | LKSDGSFFLYSKLTVDKSRWQQGNVFSCSVMHEALHNHYTQKSL |
| | | SLSPGKSEKDEL |
| 85 | PfAMA1_GKO- | IEIVERSNYMGNPWTEYMAKYDIEEVHGSGIRVDLGEDAEVAGTQY |
| | HC2.2-ER | RLPSGKCPVFGKGIIIENSNTAFLTPVATGNQYLKDGGFAFPPTEPL |
| | | MSPMTLDEMRHFYKDNKYVKNLDELTLCSRHAGNMIPDNDKNSN |
| | | YKYPAVYDDKDKKCHILYIAAQENNGPRYCNKDESKRNSMFCFRP |
| | | AKDISFQNYAYLSKNVVDNWEKVCPRKNLQNAKFGLWVDGNCEDI |
| | | PHVNEFPAIDLFECNKLVFELSASDQPKQYEQHLTDYEKIKEGFKN |
| | | KNAAMIKSAFLPTGAFKADRYKSHGKGYNWGNYNTETQKCEIFNV |
| | | KPTCLINNAAYIATTALSHPIEVENNFPCSLYKDEIMKEIERESKRIKL |
| | | NDNDDEGNKKIIAPRIFISDDKDSLKCPCDPEMVSNSACRFFVCKC |
| | | VERRAEVTSNNEVVVKEEYKDEYADIPEHKPTYDKMKAAAKGPSV |
| | | FPLAPSSKSTSGGTAALGCLVKDYFPEPVTVSWNSGALTSGVHTF |
| | | PAVLQSSGLYSLSSVVTVPSSSLGTQTYICNVNHKPSNTKVDKKVE |
| | | PKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVL |
| | | TVLHQDWLNGKEYKCKVSNKAFPAPIEKTISKAKGQPREPQVYTLP |
| | | PSRDELTKNQVSLTCLVKGFYPSDIAVEWESNGQPENNYDTTPPV |
| | | LDSDGSFFLYSDLTVDKSRWQQGNVFSCSVMHEALHNHYTQKSL |
| | | SLSPGKSEKDEL |
| 86 | PfRh2A₁₅ (3D7, aa | KKYETYVDMKTIESKYTTVMTLSEHLLEYAMDVLKANPQKPIDPKA |
| | 446-558) | NLDSEVVKLQIKINEKSNELDNAASQVKTLIIIMKSFYDIIISEKASMD |
| | | EMEKKELSLNNYIEKTDY |
| 87 | PfRh2A₁₅-HC1.2- | KKYETYVDMKTIESKYTTVMTLSEHLLEYAMDVLKANPQKPIDPKA |
| | ER | NLDSEVVKLQIKINEKSNELDNAASQVKTLIIIMKSFYDIIISEKASMD |
| | | EMEKKELSLNNYIEKTDYAAAKGPSVFPLAPSSKSTSGGTAALGCL |
| | | VKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSN |
| | | KAFPAPIEKTISKAKGQPREPQVYTLPPSRDKLTKNQVSLTCLVKG |
| | | FYPSDIAVEWESNGQPENNYKTTPPVLKSDGSFFLYSKLTVDKSR |
| | | WQQGNVFSCSVMHEALHNHYTQKSLSLSPGKSEKDEL |
| 88 | PfRh2A₁₅-HC2.2- | KKYETYVDMKTIESKYTTVMTLSEHLLEYAMDVLKANPQKPIDPKA |
| | ER | NLDSEVVKLQIKINEKSNELDNAASQVKTLIIIMKSFYDIIISEKASMD |
| | | EMEKKELSLNNYIEKTDYAAAKGPSVFPLAPSSKSTSGGTAALGCL |
| | | VKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSN |
| | | KAFPAPIEKTISKAKGQPREPQVYTLPPSRDELTKNQVSLTCLVKGF |
| | | YPSDIAVEWESNGQPENNYDTTPPVLDSDGSFFLYSDLTVDKSRW |
| | | QQGNVFSCSVMHEALHNHYTQKSLSLSPGKSEKDEL |
| 89 | PfCyRPA-HC1.2- | DSRHVFIRTELSFIKNNVPCIRDMFFIYKRELYNICLDDLKGEEDETH |
| | ER | IYVQKKVKDSWITLNDLFKETDLTGRPHIFAYVDVEEIIILLCEDEEFS |
| | | NRKKDMTCHRFYSNDGKEYNNSEITISDYILKDKLLSSYVSLPLKIE |
| | | NREYFLICGVSPYKFKDDNKKDDILCMASHDKGETWGTKIVIKYDN |
| | | YKLGVQYFFLRPYISKNDLSFHFYVGDNINNVKNVNFIECTHEKDLE |
| | | FVCSNRDFLKDNKVLQDVSTLNDEYIVSYGNDNNFAECYIFFNNEN |
| | | SILIKPEKYGNTTAGCYGGTFVKIDENRTLFIYSAAAKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSH |
| | | EDPEVKFNWY |
| | | VDGVEVHNAKTKPREEQYNSTYRVVSVLTVLHQDWLNGKEYKCK |
| | | VSNKAFPAPIEKTISKAKGQPREPQVYTLPPSRDKLTKNQVSLTCL |
| | | VKGFYPSDIAVEWESNGQPENNYKTTPPVLKSDGSFFLYSKLTVD |
| | | KSRWQQGNVFSCSVMHEALHNHYTQKSLSLSPGKSEKDEL |
| 90 | PfCyRPA-HC2.2- | DSRHVFIRTELSFIKNNVPCIRDMFFIYKRELYNICLDDLKGEEDETH |
| | ER | IYVQKKVKDSWITLNDLFKETDLTGRPHIFAYVDVEEIIILLCEDEEFS |
| | | NRKKDMTCHRFYSNDGKEYNNSEITISDYILKDKLLSSYVSLPLKIE |
| | | NREYFLICGVSPYKFKDDNKKDDILCMASHDKGETWGTKIVIKYDN |
| | | YKLGVQYFFLRPYISKNDLSFHFYVGDNINNVKNVNFIECTHEKDLE |
| | | FVCSNRDFLKDNKVLQDVSTLNDEYIVSYGNDNNFAECYIFFNNEN |
| | | SILIKPEKYGNTTAGCYGGTFVKIDENRTLFIYSAAAKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKAFPAPIEKTISKAKGQPREPQVYTLPPSRDEL |
| | | TKNQVSLTCLVKGFYPSDIAVEWESNGQPENNYDTTPPVLDSDGS |
| | | FFLYSDLTVDKSRWQQGNVFSCSVMHEALHNHYTQKSLSLSPGK |
| | | SEKDEL |
| 91 | PfSEA1-HC1.2-ER | NEDRGIYDELLENDMCDLYNLKMHDLHNLKSYDFGLSKDLLKKDIFI |
| | | YSNNLKNDDMDDDDNNNMNDIAIGENVIYENDIHENNIDDNDMYN |
| | | NYVNGNDLYINNMQDDAMDDIVYDEEEIKSFLDKLKSDISNQMNVK |
| | | NGNVEVTGNGGNEEMSYINNDENLQAFDLLDNFHMDDYGNNYND |
| | | NEEDGDGDGDDDEQKKRKQKELHNVNGKLDLSDLNELNVDDINN |
| | | NFYMSTPRKSIDERKDTECQTDFPLLDVSRNTDRTPRRKSVEVILV |
| | | EAAAKGPSVFPLAPSSKSTSGGTAALGCLVKDYFPEPVTVSWNSG |
| | | ALTSGVHTFPAVLQSSGLYSLSSVVTVPSSSLGTQTYICNVNHKPS |
| | | NTKVDKKVEPKSCDKTHTCPPCPAPELLGGPSVFLFPPKPKDTLMI |
| | | SRTPEVTCVVVDVSHEDPEVKFNWYVDGVEVHNAKTKPREEQYN |
| | | STYRVVSVLTVLHQDWLNGKEYKCKVSNKAFPAPIEKTISKAKGQP |
| | | REPQVYTLPPSRDKLTKNQVSLTCLVKGFYPSDIAVEWESNGQPE |
| | | NNYKTTPPVLKSDGSFFLYSKLTVDKSRWQQGNVFSCSVMHEAL |
| | | HNHYTQKSLSLSPGKSEKDEL |
| 92 | PfSEA1-HC2.2-ER | NEDRGIYDELLENDMCDLYNLKMHDLHNLKSYDFGLSKDLLKKDIFI |
| | | YSNNLKNDDMDDDDNNNMNDIAIGENVIYENDIHENNIDDNDMYN |
| | | NYVNGNDLYINNMQDDAMDDIVYDEEEIKSFLDKLKSDISNQMNVK |
| | | NGNVEVTGNGGNEEMSYINNDENLQAFDLLDNFHMDDYGNNYND |
| | | NEEDGDGDGDDDEQKKRKQKELHNVNGKLDLSDLNELNVDDINN |
| | | NFYMSTPRKSIDERKDTECQTDFPLLDVSRNTDRTPRRKSVEVILV |
| | | EAAAKGPSVFPLAPSSKSTSGGTAALGCLVKDYFPEPVTVSWNSG |
| | | ALTSGVHTFPAVLQSSGLYSLSSVVTVPSSSLGTQTYICNVNHKPS |
| | | NTKVDKKVEPKSCDKTHTCPPCPAPELLGGPSVFLFPPKPKDTLMI |
| | | SRTPEVTCVVVDVSHEDPEVKFNWYVDGVEVHNAKTKPREEQYN |
| | | STYRVVSVLTVLHQDWLNGKEYKCKVSNKAFPAPIEKTISKAKGQP |
| | | REPQVYTLPPSRDELTKNQVSLTCLVKGFYPSDIAVEWESNGQPE |
| | | NNYDTTPPVLDSDGSFFLYSDLTVDKSRWQQGNVFSCSVMHEAL |
| | | HNHYTQKSLSLSPGKSEKDEL |

In the following table 5 examples of further HC fusion polypeptides with modified C_{H}3 regions for Fc-heterodimerization and additional C-terminally fused (second or fourth) malaria antigens are shown.

TABLE 5
modMIA-C fusions: MIA-C fusion polypeptides with
modified CH3 regions for Fc-heterodimerization

| | | |
|---|---|---|
| 93 | Tetra_MSP1₁₉- | ISQHQCVKKQCPENSGCFRHLDEREECKCLLNYKQEGDKCVENP |
| | HC1.2- | NPTCNENNGGCDADATCTEEDSGSSRKKITCECTKPDSYPLFDGI |
| | PfCSP_TSR-ERH | FCSSSNAAVAMAISQHQCVKKQCPENSGCFRHLDEREECKCLLNY |
| | (stage and strain- | KQEGDKCVENPNPTCNENNGGCDADAKCTEEDSGSNGKKITCEC |
| | transcending) | TKPDSYPLFDGIFCSSSNAAVAMAISQHQCVKKQCPQNSGCFRHL |
| | | DEREECKCLLNYKQEGDKCVENPNPTCNENNGGCDADAKCTEED |
| | | SGSNGKKITCECTKPDSYPLFDGIFCSSSNAAVAMAISQHQCVKKQ |
| | | CPQNSGCFRHLDEREECKCLLNYKQEGDKCVENPNPTCNENNGG |
| | | CDADAKCTEEDSGSNGKKITCECTKPDSYPFFDGIFCSSSNAAAK |
| | | GPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRV |
| | | VSVLTVLHQDWLNGKEYKCKVSNKAFPAPIEKTISKAKGQPREPQV |
| | | YTLPPSRDKLTKNQVSLTCLVKGFYPSDIAVEWESNGQPENNYKT |
| | | TPPVLKSDGSFFLYSKLTVDKSRWQQGNVFSCSVMHEALHNHYT |
| | | QKSLSLSPGKHMAPSDKHIEQYLKKIQNSLSTEWSPCSVTCGNGIQ |
| | | VRIKPGSANKPKDELDYENDIEKKICKMEKCSSVFNVVNSSAAAHH |
| | | HHHHSEKDEL |
| 94 | Tetra_MSP1₁₉- | ISQHQCVKKQCPENSGCFRHLDEREECKCLLNYKQEGDKCVENP |
| | HC2.2- | NPTCNENNGGCDADATCTEEDSGSSRKKITCECTKPDSYPLFDGI |
| | PfCSP_TSR-ERH | FCSSSNAAVAMAISQHQCVKKQCPENSGCFRHLDEREECKCLLNY |
| | (stage and strain- | KQEGDKCVENPNPTCNENNGGCDADAKCTEEDSGSNGKKITCEC |
| | transcending) | TKPDSYPLFDGIFCSSSNAAVAMAISQHQCVKKQCPQNSGCFRHL |
| | | DEREECKCLLNYKQEGDKCVENPNPTCNENNGGCDADAKCTEED |
| | | SGSNGKKITCECTKPDSYPLFDGIFCSSSNAAVAMAISQHQCVKKQ |
| | | CPQNSGCFRHLDEREECKCLLNYKQEGDKCVENPNPTCNENNGG |
| | | CDADAKCTEEDSGSNGKKITCECTKPDSYPFFDGIFCSSSNAAAK |
| | | GPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRV |
| | | VSVLTVLHQDWLNGKEYKCKVSNKAFPAPIEKTISKAKGQPREPQV |
| | | YTLPPSRDELTKNQVSLTCLVKGFYPSDIAVEWESNGQPENNYDT |
| | | TPPVLDSDGSFFLYSDLTVDKSRWQQGNVFSCSVMHEALHNHYT |
| | | QKSLSLSPGKHMAPSDKHIEQYLKKIQNSLSTEWSPCSVTCGNGIQ |
| | | VRIKPGSANKPKDELDYENDIEKKICKMEKCSSVFNVVNSSAAAHH |
| | | HHHHSEKDEL |
| 95 | Tetra_MSP1₁₉- | ISQHQCVKKQCPENSGCFRHLDEREECKCLLNYKQEGDKCVENP |
| | HC1.2-PfRON2L- | NPTCNENNGGCDADATCTEEDSGSSRKKITCECTKPDSYPLFDGI |
| | ERH | FCSSSNAAVAMAISQHQCVKKQCPENSGCFRHLDEREECKCLLNY |
| | (strain- | KQEGDKCVENPNPTCNENNGGCDADAKCTEEDSGSNGKKITCEC |
| | transcending) | TKPDSYPLFDGIFCSSSNAAVAMAISQHQCVKKQCPQNSGCFRHL |
| | | DEREECKCLLNYKQEGDKCVENPNPTCNENNGGCDADAKCTEED |
| | | SGSNGKKITCECTKPDSYPLFDGIFCSSSNAAVAMAISQHQCVKKQ |
| | | CPQNSGCFRHLDEREECKCLLNYKQEGDKCVENPNPTCNENNGG |
| | | CDADAKCTEEDSGSNGKKITCECTKPDSYPFFDGIFCSSSNAAAK |
| | | GPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRV |
| | | VSVLTVLHQDWLNGKEYKCKVSNKAFPAPIEKTISKAKGQPREPQV |
| | | YTLPPSRDKLTKNQVSLTCLVKGFYPSDIAVEWESNGQPENNYKT |
| | | TPPVLKSDGSFFLYSKLTVDKSRWQQGNVFSCSVMHEALHNHYT |
| | | QKSLSLSPGKHMDITQQAKDIGAGPVASCFTTRMSPPQQICLNSVV |
| | | NTALSTSTQSAMKAAAHHHHHHSEKDEL |
| 96 | Tetra_MSP1₁₉- | ISQHQCVKKQCPENSGCFRHLDEREECKCLLNYKQEGDKCVENP |
| | HC2.2-PfRON2L- | NPTCNENNGGCDADATCTEEDSGSSRKKITCECTKPDSYPLFDGI |
| | ERH | FCSSSNAAVAMAISQHQCVKKQCPENSGCFRHLDEREECKCLLNY |
| | (stage and strain- | KQEGDKCVENPNPTCNENNGGCDADAKCTEEDSGSNGKKITCEC |
| | transcending) | TKPDSYPLFDGIFCSSSNAAVAMAISQHQCVKKQCPQNSGCFRHL |
| | | DEREECKCLLNYKQEGDKCVENPNPTCNENNGGCDADAKCTEED |
| | | SGSNGKKITCECTKPDSYPLFDGIFCSSSNAAVAMAISQHQCVKKQ |
| | | CPQNSGCFRHLDEREECKCLLNYKQEGDKCVENPNPTCNENNGG |
| | | CDADAKCTEEDSGSNGKKITCECTKPDSYPFFDGIFCSSSNAAAK |
| | | GPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRV |
| | | VSVLTVLHQDWLNGKEYKCKVSNKAFPAPIEKTISKAKGQPREPQV |
| | | YTLPPSRDELTKNQVSLTCLVKGFYPSDIAVEWESNGQPENNYDT |
| | | TPPVLDSDGSFFLYSDLTVDKSRWQQGNVFSCSVMHEALHNHYT |
| | | QKSLSLSPGKHMDITQQAKDIGAGPVASCFTTRMSPPQQICLNSVV |
| | | NTALSTSTQSAMKAAAHHHHHHSEKDEL |
| 97 | PfAMA1_GKO- | IEIVERSNYMGNPWTEYMAKYDIEEVHGSGIRVDLGEDAEVAGTQY |
| | HC1.2- | RLPSGKCPVFGKGIIIENSNTAFLTPVATGNQYLKDGGFAFPPTEPL |
| | PfCSP_TSR-ERH | MSPMTLDEMRHFYKDNKYVKNLDELTLCSRHAGNMIPDNDKNSN |
| | (stage and strain- | YKYPAVYDDKDKKCHILYIAAQENNGPRYCNKDESKRNSMFCFRP |
| | transcending) | AKDISFQNYAYLSKNVVDNWEKVCPRKNLQNAKFGLWVDGNCEDI |
| | | PHVNEFPAIDLFECNKLVFELSASDQPKQYEQHLTDYEKIKEGFKN |
| | | KNAAMIKSAFLPTGAFKADRYKSHGKGYNWGNYNTETQKCEIFNV |
| | | KPTCLINNAAYIATTALSHPIEVENNFPCSLYKDEIMKEIERESKRIKL |
| | | NDNDDEGNKKIIAPRIFISDDKDSLKCPCDPEMVSNSACRFFVCKC |
| | | VERRAEVTSNNEVVVKEEYKDEYADIPEHKPTYDKMKAAAKGPSV |
| | | FPLAPSSKSTSGGTAALGCLVKDYFPEPVTVSWNSGALTSGVHTF |
| | | PAVLQSSGLYSLSSVVTVPSSSLGTQTYICNVNHKPSNTKVDKKVE |
| | | PKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVL |
| | | TVLHQDWLNGKEYKCKVSNKAFPAPIEKTISKAKGQPREPQVYTLP |
| | | PSRDKLTKNQVSLTCLVKGFYPSDIAVEWESNGQPENNYKTTPPV |
| | | LKSDGSFFLYSKLTVDKSRWQQGNVFSCSVMHEALHNHYTQKSL |
| | | SLSPGKHMAPSDKHIEQYLKKIQNSLSTEWSPCSVTCGNGIQVRIK |
| | | PGSANKPKDELDYENDIEKKICKMEKCSSVFNVVNSSAAAHHHHH |
| | | HSEKDEL |
| 98 | PfAMA1_GKO- | IEIVERSNYMGNPWTEYMAKYDIEEVHGSGIRVDLGEDAEVAGTQY |
| | HC2.2- | RLPSGKCPVFGKGIIIENSNTAFLTPVATGNQYLKDGGFAFPPTEPL |
| | PfCSP_TSR-ERH | MSPMTLDEMRHFYKDNKYVKNLDELTLCSRHAGNMIPDNDKNSN |
| | (stage and strain- | YKYPAVYDDKDKKCHILYIAAQENNGPRYCNKDESKRNSMFCFRP |
| | transcending) | AKDISFQNYAYLSKNVVDNWEKVCPRKNLQNAKFGLWVDGNCEDI |
| | | PHVNEFPAIDLFECNKLVFELSASDQPKQYEQHLTDYEKIKEGFKN |
| | | KNAAMIKSAFLPTGAFKADRYKSHGKGYNWGNYNTETQKCEIFNV |
| | | KPTCLINNAAYIATTALSHPIEVENNFPCSLYKDEIMKEIERESKRIKL |
| | | NDNDDEGNKKIIAPRIFISDDKDSLKCPCDPEMVSNSACRFFVCKC |
| | | VERRAEVTSNNEVVVKEEYKDEYADIPEHKPTYDKMKAAAKGPSV |
| | | FPLAPSSKSTSGGTAALGCLVKDYFPEPVTVSWNSGALTSGVHTF |
| | | PAVLQSSGLYSLSSVVTVPSSSLGTQTYICNVNHKPSNTKVDKKVE |
| | | PKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVL |
| | | TVLHQDWLNGKEYKCKVSNKAFPAPIEKTISKAKGQPREPQVYTLP |
| | | PSRDELTKNQVSLTCLVKGFYPSDIAVEWESNGQPENNYDTTPPV |
| | | LDSDGSFFLYSDLTVDKSRWQQGNVFSCSVMHEALHNHYTQKSL |
| | | SLSPGKHMAPSDKHIEQYLKKIQNSLSTEWSPCSVTCGNGIQVRIK |
| | | PGSANKPKDELDYENDIEKKICKMEKCSSVFNVVNSSAAAHHHHH |
| | | HSEKDEL |
| 99 | PfRh2₁₅-HC2.2.- | KKYETYVDMKTIESKYTTVMTLSEHLLEYAMDVLKANPQKPIDPKA |
| | PfExp1 | NLDSEVVKLQIKINEKSNELDNAASQVKTLIIIMKSFYDIIISEKASMD |
| | | EMEKKELSLNNYIEKTDYAAAKGPSVFPLAPSSKSTSGGTAALGCL |
| | | VKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSN |
| | | KAFPAPIEKTISKAKGQPREPQVYTLPPSRDELTKNQVSLTCLVKGF |
| | | YPSDIAVEWESNGQPENNYDTTPPVLDSDGSFFLYSDLTVDKSRW |
| | | QQGNVFSCSVMHEALHNHYTQKSLSLSPGKHMAEKTNKGTGSGV |
| | | SSKKKNKKGSGEPLIDVHDLISDMIKKEEELVEVNKRKSKYKLATSN |
| | | TEKGRHPFKIGSSDPADNANPDADSESNGEPNADPQVTAQDVTPE |
| | | QPQGDDNNLVSGPEH |
| 100 | PfRh2₁₅-HC2.2.- | KKYETYVDMKTIESKYTTVMTLSEHLLEYAMDVLKANPQKPIDPKA |
| | PfRON2L-ERH | NLDSEVVKLQIKINEKSNELDNAASQVKTLIIIMKSFYDIIISEKASMD |
| | | EMEKKELSLNNYIEKTDYAAAKGPSVFPLAPSSKSTSGGTAALGCL |
| | | VKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSN |
| | | KAFPAPIEKTISKAKGQPREPQVYTLPPSRDELTKNQVSLTCLVKGF |
| | | YPSDIAVEWESNGQPENNYDTTPPVLDSDGSFFLYSDLTVDKSRW |
| | | QQGNVFSCSVMHEALHNHYTQKSLSLSPGKHMDITQQAKDIGAGP |
| | | VASCFTTRMSPPQQICLNSVVNTALSTSTQSAMKAAAHHHHHHSE |
| | | KDEL |
| 101 | PfCyRPA-HC1.2- | DSRHVFIRTELSFIKNNVPCIRDMFFIYKRELYNICLDDLKGEEDETH |
| | PfRON2L-ERH | IYVQKKVKDSWITLNDLFKETDLTGRPHIFAYVDVEEIIILLCEDEEFS |
| | | NRKKDMTCHRFYSNDGKEYNNSEITISDYILKDKLLSSYVSLPLKIE |
| | | NREYFLICGVSPYKFKDDNKKDDILCMASHDKGETWGTKIVIKYDN |
| | | YKLGVQYFFLRPYISKNDLSFHFYVGDNINNVKNVNFIECTHEKDLE |
| | | FVCSNRDFLKDNKVLQDVSTLNDEYIVSYGNDNNFAECYIFFNNEN |
| | | SILIKPEKYGNTTAGCYGGTFVKIDENRTLFIYSAAAKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKAFPAPIEKTISKAKGQPREPQVYTLPPSRDKL |
| | | TKNQVSLTCLVKGFYPSDIAVEWESNGQPENNYKTTPPVLKSDGS |
| | | FFLYSKLTVDKSRWQQGNVFSCSVMHEALHNHYTQKSLSLSPGK |
| | | HMAPSDKHIEQYLKKIQNSLSTEWSPCSVTCGNGIQVRIKPGSANK |
| | | PKDELDYENDIEKKICKMEKCSSVFNVVNSSAAAHHHHHHSEKDEL |
| 102 | PfCyRPA-HC2.2- | DSRHVFIRTELSFIKNNVPCIRDMFFIYKRELYNICLDDLKGEEDETH |
| | PfRON2L-ERH | IYVQKKVKDSWITLNDLFKETDLTGRPHIFAYVDVEEIIILLCEDEEFS |
| | | NRKKDMTCHRFYSNDGKEYNNSEITISDYILKDKLLSSYVSLPLKIE |
| | | NREYFLICGVSPYKFKDDNKKDDILCMASHDKGETWGTKIVIKYDN |
| | | YKLGVQYFFLRPYISKNDLSFHFYVGDNINNVKNVNFIECTHEKDLE |
| | | FVCSNRDFLKDNKVLQDVSTLNDEYIVSYGNDNNFAECYIFFNNEN |
| | | SILIKPEKYGNTTAGCYGGTFVKIDENRTLFIYSAAAKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKAFPAPIEKTISKAKGQPREPQVYTLPPSRDEL |
| | | TKNQVSLTCLVKGFYPSDIAVEWESNGQPENNYDTTPPVLDSDGS |
| | | FFLYSDLTVDKSRWQQGNVFSCSVMHEALHNHYTQKSLSLSPGK |
| | | HMDITQQAKDIGAGPVASCFTTRMSPPQQICLNSVVNTALSTSTQS |
| | | AMKAAAHHHHHHSEKDEL |
| 103 | PfSEA1-HC1.2- | NEDRGIYDELLENDMCDLYNLKMHDLHNLKSYDFGLSKDLLKKDIFI |
| | PfRON2L-ERH | YSNNLKNDDMDDDDNNNMNDIAIGENVIYENDIHENNIDDNDMYN |
| | | NYVNGNDLYINNMQDDAMDDIVYDEEEIKSFLDKLKSDISNQMNVK |
| | | NGNVEVTGNGGNEEMSYINNDENLQAFDLLDNFHMDDYGNNYND |
| | | NEEDGDGDGDDDEQKKRKQKELHNVNGKLDLSDLNELNVDDINN |
| | | NFYMSTPRKSIDERKDTECQTDFPLLDVSRNTDRTPRRKSVEVILV |
| | | EAAAKGPSVFPLAPSSKSTSGGTAALGCLVKDYFPEPVTVSWNSG |
| | | ALTSGVHTFPAVLQSSGLYSLSSVVTVPSSSLGTQTYICNVNHKPS |
| | | NTKVDKKVEPKSCDKTHTCPPCPAPELLGGPSVFLFPPKPKDTLMI |
| | | SRTPEVTCVVVDVSHEDPEVKFNWYVDGVEVHNAKTKPREEQYN |
| | | STYRVVSVLTVLHQDWLNGKEYKCKVSNKAFPAPIEKTISKAKGQP |
| | | REPQVYTLPPSRDKLTKNQVSLTCLVKGFYPSDIAVEWESNGQPE |
| | | NNYKTTPPVLKSDGSFFLYSKLTVDKSRWQQGNVFSCSVMHEAL |
| | | HNHYTQKSLSLSPGKHMAPSDKHIEQYLKKIQNSLSTEWSPCSVT |
| | | CGNGIQVRIKPGSANKPKDELDYENDIEKKICKMEKCSSVFNVVNS |
| | | SAAAHHHHHHSEKDEL |
| 104 | PfSEA1-HC2.2- | NEDRGIYDELLENDMCDLYNLKMHDLHNLKSYDFGLSKDLLKKDIFI |
| | PfRON2L-ERH | YSNNLKNDDMDDDDNNNMNDIAIGENVIYENDIHENNIDDNDMYN |
| | | NYVNGNDLYINNMQDDAMDDIVYDEEEIKSFLDKLKSDISNQMNVK |
| | | NGNVEVTGNGGNEEMSYINNDENLQAFDLLDNFHMDDYGNNYND |
| | | NEEDGDGDGDDDEQKKRKQKELHNVNGKLDLSDLNELNVDDINN |
| | | NFYMSTPRKSIDERKDTECQTDFPLLDVSRNTDRTPRRKSVEVILV |
| | | EAAAKGPSVFPLAPSSKSTSGGTAALGCLVKDYFPEPVTVSWNSG |
| | | ALTSGVHTFPAVLQSSGLYSLSSVVTVPSSSLGTQTYICNVNHKPS |
| | | NTKVDKKVEPKSCDKTHTCPPCPAPELLGGPSVFLFPPKPKDTLMI |
| | | SRTPEVTCVVVDVSHEDPEVKFNWYVDGVEVHNAKTKPREEQYN |
| | | STYRVVSVLTVLHQDWLNGKEYKCKVSNKAFPAPIEKTISKAKGQP |
| | | REPQVYTLPPSRDELTKNQVSLTCLVKGFYPSDIAVEWESNGQPE |
| | | NNYDTTPPVLDSDGSFFLYSDLTVDKSRWQQGNVFSCSVMHEAL |
| | | HNHYTQKSLSLSPGKHMDITQQAKDIGAGPVASCFTTRMSPPQQI |
| | | CLNSVVNTALSTSTQSAMKAAAHHHHHHSEKDEL |
| 105 | MPT64-HC1 | APKTYCQELKGTDTGQACQIQMSDPAYNINISLPSYYPDQKSLENY |
| | | IAQTRDKFLSAATSSTPREAPYELNITSATYQSAIPPRGTQAVVLKV |
| | | YQNAGGTHPTTTYKAFDWDQAYRKPITYDTLWQADTDPLPVVFPI |
| | | VQGELSKQTGQQVSIAPNAGLDPVNYQNFAVTNDGVIFFFNPGELL |
| | | PEAAGPTQVLVPRSAIDSMLAAAAKGPSVFPLAPSSKSTSGGTAAL |
| | | GCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAP |
| | | ELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCK |
| | | VSNKAFPAPIEKTISKAKGQPREPQVYTLPPSRDKLTKNQVSLTCLV |
| | | KGFYPSDIAVEWESNGQPENNYKTTPPVLKSDGSFFLYSKLTVDK |
| | | SRWQQGNVFSCSVMHEALHNHYTQKSLSLSPGKSEKDEL |
| 106 | HVR1-HC2 | QTTVVGGSQSHTVRGLTSLFSPGASQNAAAKGPSVFPLAPSSKST |
| | | SGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTC |
| | | PPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNG |
| | | KEYKCKVSNKAFPAPIEKTISKAKGQPREPQVYTLPPSRDELTKNQ |
| | | VSLTCLVKGFYPSDIAVEWESNGQPENNYDTTPPVLDSDGSFFLY |
| | | SDLTVDKSRWQQGNVFSCSVMHEALHNHYTQKSLSLSPGKSEKD |
| | | EL |
| 107 | CLCT-LC + eldkwa | FRGNNGHDSSSSLYGGSQFIEQLDNSFTSAFLESQSMNKIGDDLA |
| | | ETISNELVSVLQKNSPTFLESSFDIKSEVKKHAKSMLKELIKVGLPSF |
| | | ENLVAENVKPPKVDPATYGIIVPVL |
| | | TSLFNKVETAVGAKVSDEIWNYNSPDVSESEESLSDDFFDAAGPN |
| | | ANPNANPNANPNANPNANPNANPNANPNANPNANPNANPNANPN |
| | | ANPNANPNANPNANPNANPNANPNANPNKNNQGNGQGHNMPND |
| | | PNRNVDENANANSAVKNNNNEEPSDKHIEQYLKKIQNSLSTEWSP |
| | | CSVTCGNGIQVRIKPGSANKPKDELDYENDIEKKICKMEKCSSVFN |
| | | VVNSAAVAMAEKTASCGVWDEWSPCSVTCGKGTRSRKREILHEG |
| | | CTSELQEQCEEERCLPKAAAPSVFIFPPSDEQLKSGTASVVCLLNN |
| | | FYPREAKVQWKVDNALQSGNSQESVTEQDSKDSTYSLSSTLTLSK |
| | | ADYEKHKVYACEVTHQGLSSPVTKSFNRGECELDKWA |

B) LC Fusion Polypeptides

As mentioned above, in some advantageous embodiments an isolated protein complexes of the present disclosure comprise at least two recombinant fusion protein units, wherein the second fusion protein unit comprising an immunoglobulin light chain constant domain C_{L}, and a second antigen, wherein said second antigen is linked N-terminal and/or C-terminal to the C_{L}-domain (LC fusion polypeptide unit 1, LC unit 1).

In some advantageous embodiments, the isolated protein complex according to the present disclosure comprises a fourth recombinant fusion protein unit comprising an immunoglobulin light chain constant domain C_{L}, and a fourth antigen, wherein said fourth antigen is fused N- or C-terminal to the C_{L}-domain (second LC fusion polypeptide unit 2, LC unit 2).

In the following the first and the second LC fusion polypeptide will be referred to as LC fusion polypeptide.

Typically, in a LC fusion polypeptide (LC unit land/or LC unit 2)) comprised in the isolated protein complex according to the present disclosure comprises an amino acid sequence of a IgG, IgM, or IgA immunoglobulin light chain constant domain; or variant thereof. In particular, the immunoglobulin light chain constant domain comprises an amino acid sequence of a mammalian light chain constant domain, preferably a human light chain constant domain; or variant thereof. Preferably, each of the immunoglobulin light chain constant domains comprise an amino acid sequence or a variant thereof of a IgG light chain constant domain, preferably a human kappa or lamda light chain.

The phrase “light chain” includes an immunoglobulin light chain sequence from any organism, and unless otherwise specified includes human κ and λ light chains and a VpreB, as well as surrogate light chains. Light chain variable (V_{L}) domains typically include three light chain CDRs and four framework (FR) regions, unless otherwise specified.

Generally, a full-length light chain includes, from amino terminus to carboxyl terminus, a VL domain that includes FR1-CDR1-FR2-CDR2-FR3-CDR3-FR4, and a light chain constant domain of kappa or lamda isotype. Light chains include those, e.g., that do not selectively bind either a first or a second epitope selectively bound by the epitope-binding protein in which they appear. Light chains also include those that bind and recognize, or assist the heavy chain with binding and recognizing, one or more epitopes selectively bound by the epitope-binding protein in which they appear.

The term “immunoglobulin light chain constant domain” is a polypeptide derived from a native immunoglobulin light chain like the kappa (κ) chain, encoded by the immunoglobulin kappa locus (IGK@) or the lambda (λ) chain, encoded by the immunoglobulin lambda locus (IGL@), or variant or fragment thereof (see e.g. Kapelski et al., Malaria Journal 2015, 14:50). According to the Kabat numbering scheme the positions may be for hLCkappa: EU/Kabat numbering: 111-214). Examples of antibody constant regions are shown in the IMGT Repertoire database of INTERNATIONAL IMMUNOGENETICS INFORMATION SYSTEM (http://www.imgt.org), a global reference in immunogenetics and immunoinformatics.

In some advantageous embodiments, the second fusion protein comprises an immunoglobulin light chain constant domain C_{L} derived from an immunoglobulin light chain sequence without a V_{L} domain.

In some advantageous embodiments, the C_{L}-domain is a human kappa light chain or a lambda light chain, in particular a C_{L}-domain comprising the amino acid sequence of SEQ ID NO. 50.

Typically, a LC fusion polypeptide (LC fusion polypeptide and/or second LC fusion polypeptide) comprises an amino acid sequence of a IgG, IgM, or IgA light chain constant region; or variant thereof. Typically, each of the immunoglobulin light chain constant regions comprises an amino acid sequence of a mammalian light chain constant region, preferably a human light chain constant region; or variant thereof. Suitably, the immunoglobulin light chain constant region comprises an amino acid sequence of an IgG light chain constant region, preferably a human IgG. Suitable human IgG subtypes are IgG1, IgG2, IgG3 and lgG4, although igG1 or lgG3 are preferred.

According to the present disclosure, an antigen may be fused N-terminal and/or C-terminal to the immunoglobulin light chain constant domain of the second fusion protein (LC fusion unit 1). The antigen may be may be linked either directly or indirectly N-terminal and/or C-terminal to the C_{L}. In some embodiments, th second antigen is linked N-terminal to the C_{L} and another antigen is linked C-terminal to the C_{L}. The other antigen may derived from the same or from a different polypeptide, for example the antigen linked N-terminal to the C_{L} comprises the identical or at least 85% identical amino acid sequence as the antigen linked C-terminal to the C_{L}. In some advantageous embodiments, the antigen linked N-terminal to the C_{L} comprises a different amino acid sequence as the antigen linked C-terminal to the C_{L}. Therefore, a higher variability is given in the protein complex.

In the following table 6, examples of HC fusion polypeptides are shown.

TABLE 6
MIA LC fusions: Malaria Immunoassemblin
light chain fusion polypeptides

| | | |
|---|---|---|
| 50 | human LC kappa | APSVFIFPPSDEQLKSGTASVVCLLNNFYPREAKVQWKVDNALQS |
| | constant domain | GNSQESVTEQDSKDSTYSLSSTLTLSKADYEKHKVYACEVTHQGL |
| | (LC) | SSPVTKSFNRGEC |
| 51 | PfMSP1₁₉_3D7-LC | ISQHQCVKKQCPENSGCFRHLDEREECKCLLNYKQEGDKCVENP |
| | | NPTCNENNGGCDADATCTEEDSGSSRKKITCECTKPDSYPLFDGI |
| | | FCSSSNAAAPSVFIFPPSDEQLKSGTASVVCLLNNFYPREAKVQW |
| | | KVDNALQSGNSQESVTEQDSKDSTYSLSSTLTLSKADYEKHKVYA |
| | | CEVTHQGLSSPVTKSFNRGEC |
| 52 | Tetra_MSP1₁₉-LC | ISQHQCVKKQCPENSGCFRHLDEREECKCLLNYKQEGDKCVENP |
| | (strain- | NPTCNENNGGCDADATCTEEDSGSSRKKITCECTKPDSYPLFDGI |
| | transcending) | FCSSSNAAVAMAISQHQCVKKQCPENSGCFRHLDEREECKCLLNY |
| | | KQEGDKCVENPNPTCNENNGGCDADAKCTEEDSGSNGKKITCEC |
| | | TKPDSYPLFDGIFCSSSNAAVAMAISQHQCVKKQCPQNSGCFRHL |
| | | DEREECKCLLNYKQEGDKCVENPNPTCNENNGGCDADAKCTEED |
| | | SGSNGKKITCECTKPDSYPLFDGIFCSSSNAAVAMAISQHQCVKKQ |
| | | CPQNSGCFRHLDEREECKCLLNYKQEGDKCVENPNPTCNENNGG |
| | | CDADAKCTEEDSGSNGKKITCECTKPDSYPFFDGIFCSSSNAAAPS |
| | | VFIFPPSDEQLKSGTASVVCLLNNFYPREAKVQWKVDNALQSGNS |
| | | QESVTEQDSKDSTYSLSSTLTLSKADYEKHKVYACEVTHQGLSSP |
| | | VTKSFNRGEC |
| 53 | Pfs25_FKO-LC | VTVDTVCKRGFLIQMSGHLECKCENDLVLVNEETCEEKVLKCDEKT |
| | | VNKPCGDFSKCIKIDGNPVSYACKCNLGYDMVNNVCIPNECKNVA |
| | | CGNGKCILDTSNPVKTGVCSCNIGKVPNVQDQKCSKDGETKCSLK |
| | | CLKENEACKAVDGIYKCDCKDGFIIDNEASICTAAAPSVFIFPPSDE |
| | | QLKSGTASVVCLLNNFYPREAKVQWKVDNALQSGNSQESVTEQD |
| | | SKDSTYSLSSTLTLSKADYEKHKVYACEVTHQGLSSPVTKSFNRGEC |
| 54 | Pfs25_SHKO-LC | VTVDTVCKRGFLIQMSGHLECKCENDTVLVNEETCEEKVLKCDEK |
| | | TVNKPCGDFSKCIKIDGNPVSYACKCNLGYDMVNNVCIPNECKNVT |
| | | CGNGKCILDTSNPVKTGVCSCNIGKVPNVQDQKCSKDGETKCSLK |
| | | CLKENETCKAVDGIYKCDCKDGFIIDNEASICTAAAPSVFIFPPSDE |
| | | QLKSGTASVVCLLNNFYPREAKVQWKVDNALQSGNSQESVTEQD |
| | | SKDSTYSLSSTLTLSKADYEKHKVYACEVTHQGLSSPVTKSFNRGEC |
| 55 | Pfs28-LC | VTENTICKYGYLIQMSNHYECKCIEGYVLINEDTCGKKVVCDKVEN |
| | | SFKACDEYAYCFDLGNKNNEKQIKCMCRTEYTLTAGVCVPNVCRD |
| | | KVCGKGKCIVDPANSLTHTCSCNIGTILNQNKLCDIQGDTPCSLKCA |
| | | ENEVCTLEGNYYTCKEDPSSNGGGNTVDQASAAAPSVFIFPPSDE |
| | | QLKSGTASVVCLLNNFYPREAKVQWKVDNALQSGNSQESVTEQD |
| | | SKDSTYSLSSTLTLSKADYEKHKVYACEVTHQGLSSPVTKSFNRGEC |
| 56 | PfCSP_TSR-LC | PSDKHIEQYLKKIQNSLSTEWSPCSVTCGNGIQVRIKPGSANKPKD |
| | | ELDYENDIEKKICKMEKCSSVFNVVNSSAAAPSVFIFPPSDEQLKSG |
| | | TASVVCLLNNFYPREAKVQWKVDNALQSGNSQESVTEQDSKDST |
| | | YSLSSTLTLSKADYEKHKVYACEVTHQGLSSPVTKSFNRGEC |
| 57 | PfMTRAP_TSR-LC | THDTCDEWSEWSACTHGISTRKCLSDSSIKDETLVCTKCDKWGE |
| | | WSECKDGRMHRKVLNCPFIKEEQECDVNNEAAAPSVFIFPPSDEQ |
| | | LKSGTASVVCLLNNFYPREAKVQWKVDNALQSGNSQESVTEQDS |
| | | KDSTYSLSSTLTLSKADYEKHKVYACEVTHQGLSSPVTKSFNRGEC |
| 58 | PfCTMT-LC | PSDKHIEQYLKKIQNSLSTEWSPCSVTCGNGIQVRIKPGSANKPKD |
| | (stage- | ELDYENDIEKKICKMEKCSSVFNVVNSSAAVAMAEKTASCGVWDE |
| | transcending) | WSPCSVTCGKGTRSRKREILHEGCTSELQEQCEEERCLPKAAVA |
| | | MATHDTCDEWSEWSACTHGISTRKCLSDSSIKDETLVCTKCDKWG |
| | | EWSECKDGRMHRKVLNCPFIKEEQECDVNNEAAVAMAFYSEWGE |
| | | WSNCAMDCDHPDNVQIRERECIHPSGDCFKGDLKESRPCIIPLPPC |
| | | NELFSHKDNSAFKAAAPSVFIFPPSDEQLKSGTASVVCLLNNFYPR |
| | | EAKVQWKVDNALQSGNSQESVTEQDSKDSTYSLSSTLTLSKADYE |
| | | KHKVYACEVTHQGLSSPVTKSFNRGEC |
| 59 | PfMSP3A_Nterm- | SKEIVKKYNLNLRNAILNNNSQIENEENVNTTITGNDFSGGEFLWPG |
| | LC | YTEELKAKKASEDAEKAANDAENASKEAEEAAKEAVNLKESDKSY |
| | | TKAKEAATAASKAKKAVETALKAKDDAEKSSKADSISTKTKEYAEK |
| | | AKNAYEKAKNAYQKANQAVLKAKEASSYDYILGWEFGGGVPEHKK |
| | | EENMLSHLYVSSKDKENIAKENDDVLDEKEEEAEETEEEELEEKNE |
| | | EETESEISEDEEEEEEEEKEEENDKKKEQEKEQSNENNDQKKDME |
| | | AQNLISKNQNNNEKNVKEAAESIMKTLAGLIKGNNQIDSTLKDLVEE |
| | | LSKYFKNHAAAPSVFIFPPSDEQLKSGTASVVCLLNNFYPREAKVQ |
| | | WKVDNALQSGNSQESVTEQDSKDSTYSLSSTLTLSKADYEKHKVY |
| | | ACEVTHQGLSSPVTKSFNRGEC |
| 60 | PfMSP3B_Nterm- | SKEIVKKYNLNLRNAILNNNSQIENEENDIKYELNEQNDENVNTPIV |
| | LC | GNMEFGEGFSADDQKDIEAYKKAKQASQDAEQAAKDAENAAKDA |
| | | EEAAKDAEKLKESDESYTKAKEACTAASKAKKAVETALKAKDDAET |
| | | ALKTSETPEKPSRINLFSRKTKEYAEKAKNAYEKAKNAYQKANQAV |
| | | LKAKEASSYDYILGWEFGGGVPEHKKEENMLSHLYVSSKDKENIAK |
| | | ENDDVLDEKEEEAEETEEEELEEKNEEETESEISEDEEEEEEEEKE |
| | | EENDKKKEQEKEQSNENNDQKKDMEAQNLISKNQNNNEKNVKEA |
| | | AESIMKTLAGLIKGNNQIDSTLKDLVEELSKYFKNHAAAPSVFIFPPS |
| | | DEQLKSGTASVVCLLNNFYPREAKVQWKVDNALQSGNSQESVTE |
| | | QDSKDSTYSLSSTLTLSKADYEKHKVYACEVTHQGLSSPVTKSFN |
| | | RGEC |
| 61 | PfMSP6₃₆-LC | NGLTGATENIAQVVQANSETNKNPTSHSNSTTTSLNNNILGWEFG |
| | | GGAPQNGAAEDKKTEYLLEQIKIPSWDRNNIPDENEQVIEDPQEDN |
| | | KDEDEDEETETENLETEDDNNEEIEENEEDDIDEESVEEKEEEEEK |
| | | KEEEEKKEEKKEEKKPDNEITNEVKEEQKYSSPSDINAQNLISNKN |
| | | KKNDETKKTAENIVKTLVGLFNEKNEIDSTINNLVQEMIHLFSSKSR |
| | | WSAAAPSVFIFPPSDEQLKSGTASVVCLLNNFYPREAKVQWKVDN |
| | | ALQSGNSQESVTEQDSKDSTYSLSSTLTLSKADYEKHKVYACEVT |
| | | HQGLSSPVTKSFNRGEC |
| 62 | PfMSP7₂₂-LC | SETDTQSKNEQEISTQGQEVQKPAQGGESTFQKDLDKKLYNLGDV |
| | | FNHVVDISNKENKINLDEHDKKYTDFKKEYEDFVLNSKEYDIIKNLII |
| | | MFGQEDNKAKNGKTDIVSEAKHITEIFIKLFKDKEYHEQFKNYIYGV |
| | | YSYAKQNSHLSEKKIKQEEEYKKFLEYSFNLLNTAAAPSVFIFPPSD |
| | | EQLKSGTASVVCLLNNFYPREAKVQWKVDNALQSGNSQESVTEQ |
| | | DSKDSTYSLSSTLTLSKADYEKHKVYACEVTHQGLSSPVTKSFNR |
| | | GEC |
| 63 | PfExp1-LC | EKTNKGTGSGVSSKKKNKKGSGEPLIDVHDLISDMIKKEEELVEVN |
| | | KRKSKYKLATSNTEKGRHPFKIGSSDPADNANPDADSESNGEPNA |
| | | DPQVTAQDVTPEQPQGDDNNLVSGPEHAAAPSVFIFPPSDEQLKS |
| | | GTASVVCLLNNFYPREAKVQWKVDNALQSGNSQESVTEQDSKDS |
| | | TYSLSSTLTLSKADYEKHKVYACEVTHQGLSSPVTKSFNRGEC |
| 64 | PfEBA175_F2-LC | DKNSVDTNTKVWECKKPYKLSTKDVCVPPRRQELCLGNIDRIYDK |
| | | NLLMIKEHILAIAIYESRILKRKYKNKDDKEVCKIINKAFADIRDIIGGT |
| | | DYWNDLSNRKLVGKINTNSNYVHRNKQNDKLFRDEWWKVIKKDV |
| | | WNVISWVFKDKTVCKEDDIENIPQFFRWFSEWGDDYCQDKTKMIE |
| | | TLKVECKEKPCEDDNCKRKCNSYKEWISKKKEEYNKQAKQYQEY |
| | | QKGNNYKMYSEFKSIKPEVYLKKYSEKCSNLNFEDEFKEELHSDYK |
| | | NKCTMCPEVAAAPSVFIFPPSDEQLKSGTASVVCLLNNFYPREAKV |
| | | QWKVDNALQSGNSQESVTEQDSKDSTYSLSSTLTLSKADYEKHKV |
| | | YACEVTHQGLSSPVTKSFNRGEC |
| 65 | PfAMA1_GKO-LC | IEIVERSNYMGNPWTEYMAKYDIEEVHGSGIRVDLGEDAEVAGTQY |
| | | RLPSGKCPVFGKGIIIENSNTAFLTPVATGNQYLKDGGFAFPPTEPL |
| | | MSPMTLDEMRHFYKDNKYVKNLDELTLCSRHAGNMIPDNDKNSN |
| | | YKYPAVYDDKDKKCHILYIAAQENNGPRYCNKDESKRNSMFCFRP |
| | | AKDISFQNYAYLSKNVVDNWEKVCPRKNLQNAKFGLWVDGNCEDI |
| | | PHVNEFPAIDLFECNKLVFELSASDQPKQYEQHLTDYEKIKEGFKN |
| | | KNAAMIKSAFLPTGAFKADRYKSHGKGYNWGNYNTETQKCEIFNV |
| | | KPTCLINNAAYIATTALSHPIEVENNFPCSLYKDEIMKEIER |
| | | ESKRIKLNDNDDEGNKKIIAPRIFISDDKDSLKCPCDPEMVSNSACR |
| | | FFVCKCVERRAEVTSNNEVVVKEEYKDEYADIPEHKPTYDKMKAA |
| | | APSVFIFPPSDEQLKSGTASVVCLLNNFYPREAKVQWKVDNALQS |
| | | GNSQESVTEQDSKDSTYSLSSTLTLSKADYEKHKVYACEVTHQGL |
| | | SSPVTKSFNRGEC |
| 66 | PfRON2L-LC | MDITQQAKDIGAGPVASCFTTRMSPPQQICLNSVVNTALSTSTQSA |
| | | MKAAAPSVFIFPPSDEQLKSGTASVVCLLNNFYPREAKVQWKVDN |
| | | ALQSGNSQESVTEQDSKDSTYSLSSTLTLSKADYEKHKVYACEVT |
| | | HQGLSSPVTKSFNRGEC |

### Embodiments of the IA Protein Complex According to the Present Disclosure

In some advantageous embodiments, the isolated immunoassemblin (IA) protein complexes according to the present disclosure are suitable as malaria vaccines comprising at least two recombinant fusion protein units, wherein:
a) the first fusion protein unit comprises the immunoglobulin heavy chain constant domains C_{H}1 and C_{H}3 and a first antigen, wherein said first antigen is linked N-terminal and/or C-terminal to at least one of the immunoglobulin heavy chain constant domains (HC fusion polypeptide unit 1, HC unit 1); and
b) a second fusion protein unit comprising an immunoglobulin light chain constant domain C_{L}, and a second antigen, wherein said second antigen is linked N-terminal and/or C-terminal to the C_{L}-domain (LC fusion polypeptide unit 1, LC unit 1), and wherein
c) said first and said second antigen comprises different amino acid sequences, and wherein the antigens are Pfs25 FKO, AMA1 GKO and CSP_TSR GKO.

In some advantageous embodiments, the isolated immunoassemblin (IA) protein complexes according to the present disclosure comprises a HC unit 1 comprising the antigens AMA1 GKO and CSP_TSR GKO, wherein the AMA1 GKO is N-terminal fused to the C_{H}1-domain and CSP_TSR GKO is C-terminal fused to the C_{H}3-domain, and said second fusion protein comprises the antigen Pfs25 FKO, wherein the Pfs25 FKO is N-terminal fused to the C_{L}-domain.

In some advantageous embodiments, the isolated immunoassemblin (IA) protein complexes according to the present disclosure comprises
a) a first fusion protein unit (HC unit 1) having an amino acid sequence selected from the group of SEQ ID NO. 33 to SEQ ID NO. 52, SEQ ID NO. 67 to SEQ ID NO. 75 and SEQ ID NO. 78 to SEQ ID NO. 104), and
b) a second fusion protein unit (LC unit 1 having an amino acid sequence selected from the group of SEQ ID NO. 51 to SEQ ID NO. 66).

In some advantageous embodiments, the isolated immunoassemblin (IA) protein complexes according to the present disclosure comprises two recombinant fusion proteins having the amino acid sequences of
a) first fusion protein unit (HC unit 1): SEQ ID NO. 97
b) second fusion protein unit (LC unit 1): SEQ ID NO. 53

In some further advantageous embodiments, the isolated immunoassemblin (IA) protein complexes according to the present disclosure comprises a FC-receptor binding portion, wherein the Fc receptor binding portion comprised in the first fusion protein unit having an amino acid sequence that varies from the amino acid sequence of SEQ ID NO: 32 with
a) at least two substitutions as compared with SEQ ID NO: 32, and wherein the substitutions occurs at position Glu356 and Asp399 of SEQ ID NO: 32, wherein
b) the amino acids at position Glu356 and Asp399 are substituted with positive charged amino acids; and wherein
c) the Fc receptor binding portion comprised in the third fusion protein having an amino acid sequence that varies from the amino acid sequence of SEQ ID NO: 32 with
d) at least two substitutions as compared with SEQ ID NO: 32, and wherein the substitutions occurs at position Lys392 and Lys 409 of SEQ ID NO: 32, and wherein
e) the amino acids at position Lys392 and Lys 409 are substituted with negative charged amino acids.

In some advantageous embodiments, the Fc receptor binding portion comprised in the first and/or third fusion protein unit comprises the amino acid sequence of SEQ ID NO: 76 or SEQ ID NO: 77.

In some further advantageous embodiments, the isolated IA protein complexes according to the present disclosure comprises:
a) a first fusion protein unit (HC unit 1) comprises the antigens AMA1 GKO and CSP_TSR GKO, wherein the AMA1 GKO is N-terminal fused to the C_{H}1-domain and CSP_TSR GKO is C-terminal fused to the C_{H}3-domain, and wherein the Fc receptor binding portion comprised in the first fusion protein having an amino acid sequence that varies from the amino acid sequence of SEQ ID NO: 32 with at least two substitutions as compared with SEQ ID NO: 32, and wherein the substitutions occurs at position Glu356 and Asp399 of SEQ ID NO: 32, wherein the amino acids at position Glu356 and Asp399 are substituted with positive charged amino acids; and wherein
b) a second fusion protein unit (LC unit 1) comprises the antigen Pfs25 FKO, wherein Pfs25 FKO is N-terminal fused to the C_{L}-domain, and wherein
c) a third fusion protein unit (HC unit 2) comprises the antigen Rh2 GKO, that is N-terminally fused to the C_{H}1-domain, and wherein the Fc receptor binding portion comprised in the first fusion protein having an amino acid sequence that varies from the amino acid sequence of SEQ ID NO: 32 with at least two substitutions as compared with SEQ ID NO: 32, and wherein the substitutions occurs at position Lys392 and Lys 409 of SEQ ID NO: 32.

In some further advantageous embodiments, the isolated IA protein complexes according to the present disclosure comprises:
a) a first fusion protein unit (HC unit 1): having an amino acid sequence selected from the group of SEQ ID NO. 33 to SEQ ID NO. 52, SEQ ID NO. 67 to SEQ ID NO. 75 and SEQ ID NO. 78 to SEQ ID NO. 104), and
b) a second fusion protein unit (LC unit 1) having an amino acid sequence selected from the group of SEQ ID NO. 51 to SEQ ID NO. 66), and
c) a third fusion protein unit (HC unit 22) having an amino acid sequence selected from the group of SEQ ID NO. 33 to SEQ ID NO. 52, SEQ ID NO. 67 to SEQ ID NO. 75 and SEQ ID NO. 78 to SEQ ID NO. 104).

In some further advantageous embodiments, the isolated IA protein complexes according to the present disclosure comprises:
a) a first fusion protein unit: (HC unit 1) SEQ ID NO. 97
b) a second fusion protein unit (LC unit 1): SEQ ID NO. 53
c) a third fusion protein unit (HC unit 2): SEQ ID NO. 88

In some further advantageous embodiments, the isolated IA protein complexes according to the present disclosure comprises four fusion protein units, wherein the fourth fusion protein unit (LC unit 2) is identical to the second fusion protein unit (LC unit 1) comprising the antigen Pfs25 FKO, wherein Pfs25 FKO is N-terminal fused to the C_{L}-domain of the fourth fusion protein unit.

In some further advantageous embodiments, the isolated IA protein complexes according to the present disclosure comprises:
a) a first fusion protein unit (HC unit 1) having an amino acid sequence selected from the group of SEQ ID NO. 33 to SEQ ID NO. 52, SEQ ID NO. 67 to SEQ ID NO. 75 and SEQ ID NO. 78 to SEQ ID NO. 104), and
b) a second fusion protein unit (LC unit 1) having an amino acid sequence selected from the group of SEQ ID NO. 51 to SEQ ID NO. 66), and
c) a third fusion protein unit (HC unit 2) having an amino acid sequence selected from the group of SEQ ID NO. 33 to SEQ ID NO. 52, SEQ ID NO. 67 to SEQ ID NO. 75 and SEQ ID NO. 78 to SEQ ID NO. 104), and
d) a fourth fusion protein unit (LC unit 2) having an amino acid sequence selected from the group of SEQ ID NO. 51 to SEQ ID NO. 66).

In some further advantageous embodiments, the isolated IA protein complexes according to the present disclosure comprises:
a) a first fusion protein unit (HC unit 1): SEQ ID NO. 97, and
b) a second fusion protein unit (LC unit 1): SEQ ID NO. 53, and
c) a third fusion protein unit (HC unit 2): SEQ ID NO. 88, SEQ ID NO. 99 or SEQ ID NO. 100, and
d) a fourth fusion protein unit (LC unit 2): SEQ ID NO. 53.

Furthermore, the present disclosure relates to nucleic acid molecules or nucleic acids encoding the IA protein complexes or parts thereof, in particular said recombinant fusion protein units according to the present disclosure as well as to vectors comprising the nucleic acid molecule and host cells comprising a nucleic acid molecule encoding said recombinant fusion protein units or a vector. The disclosure pertains also to methods of manufacturing said IA protein complexes and/or fusion protein units in a recombinant expression system.

Therefore, the present disclosure relates also to nucleic acid molecules comprising a coding portion encoding one or more fusion protein units according to the present disclosure, as well as expression vectors comprising at least one of said nucleotide molecules and host cells comprising at least one of said vectors or at least one of said nucleic acid molecules. In particular, said host cells comprise at least one or preferably all nucleic acid molecules encoding the fusion proteins units comprised in an isolated IA protein complex according to the present disclosure.

To express a fusion proteins (fusion protein units) according to the present disclosure in a recombinant expression system, a DNA encoding the fusion protein or parts thereof, may be inserted into an expression vector such that the gene is operably linked to transcriptional and translational control sequences. In this context, the term “operably linked” means that a protein gene is ligated into a vector such that transcriptional and translational control sequences within the vector serve their intended function of regulating the transcription and translation of the protein gene. The expression vector and expression control sequences are chosen to be compatible with the expression host cell used. The isolated protein domain sequences are typically inserted into the same expression vector. The protein genes are inserted into the expression vector by standard methods. Additionally, the recombinant expression vector can encode a signal peptide that facilitates co-translational translocation of the nascent polypeptide chain into the endoplasmic reticulum (ER). The folded polypeptide (recombinant fusion protein according to this disclosure) may be secreted from a host cell or may be retained within the host cell. Intracellular retention or targeting can be achieved by the use of an appropriate targeting peptide such as C-terminal KDEL-tag for ER retrieval.

In general, those skilled in the art are well able to construct vectors and design protocols for recombinant gene expression (Sambrook, Maniatis et al. 1989, or later editions of this work, Ausubel 1992).

The term “vector” includes a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g. bacterial vectors having a bacterial origin of replication and episomal mammalian vectors). Other vectors (e.g. non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression system” or “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g. replication-defective retroviruses, adenoviruses and adeno-associated viruses), which serve equivalent functions.

The present disclosure is also directed to a host cell with a vector comprising the recombinant fusion proteins according to the present disclosure. The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Host cells include progeny of a single host cell, and the progeny may not necessarily be completely identical (in morphology or in total DNA complement) to the original parent cell due to natural, accidental, or deliberate mutation and/or change. A host cell includes a cell transfected or infected in vivo or in vitro with a recombinant vector or a polynucleotide of the present disclosure. A host cell, which comprises a recombinant vector of the disclosure, may also be referred to as a “recombinant host cell”.

The term “host cell(s)” refers to cell(s), which may be used in a process for purifying a recombinant protein in accordance with the present disclosure. Such host cells carry the protein of interest (POI). A host cell may also be referred to as a protein-expressing cell. A host cell, according to the present invention, may be, but is not limited to, prokaryotic cells, eukaryotic cells, archaebacteria, bacterial cells, insect cells, yeast, mammal cells, and/or plant cells. Bacteria envisioned as host cells can be either gram-negative or gram-positive, e.g. Escherichia coli, Erwinia sp., Klebsellia sp., Lactobacillus sp., Bacillus subtilis or Pseudomonas fluorescens. Typical yeast host cells are selected from the group consisting of Saccharomyces cerevisiae, Hansenula polymorpha and Pichia pastoris. In some advantageous embodiments, the host cell is a HEK293 cell, such as a HEK293T cell or a HEK293-6E cell.

A wide variety of host expression systems can be used to express an IA protein complex or the fusion protein units comprised in the IA protein complex including prokaryotic (bacterial) and eukaryotic expression systems (such as yeast, baculoviral, plant, mammalian and other animal cells, transgenic animals, and hybridoma cells), as well as phage display expression systems. An example of a suitable bacterial expression vector is pUCI 19 (Sfi), and a suitable eukaryotic expression vector is a modified pcDNA3.1 vector with a weakened DHFR selection system. Another example for a suitable eukaryotic expression vector is a modified pMS vector carrying a zeocin resistance and an IRES site (Stocker et al. 2003). Another example for a suitable expression vector is the pTT vector system, carrying an improved CMV expression cassette as well as an oriP site for enhanced replication in HEK 293-6E cells (Durocher et al. 2002). An example of the plant expression vector is the pTRAkt, which is electroporated into agrobacteria and subsequently infiltrated into tobacco plants (Boes et al. 2011). Other antibody or antibody-like expression systems are also known in the art and are contemplated herein.

Furthermore, an EBV-transformed human lymphoblastoid B cell line may be used as an expression system or the antibodies or binding-portions thereof can be expressed in a cell-free protein synthesis system, for example derived from an E. coli extract.

In advantageous embodiments, the host cell is a Nicotiana benthamiana plant cell, Nicotiana tabacum plant cell or BY2 cells thereof, if mammalian, it is preferably a CHO, COS, NSO or 293 cell, if yeast, it is preferably Pichia pastoris.

Plants for use in accordance with the present disclosure include Angiosperms, Bryophytes (e g, Hepaticae, Musci, etc), Ptepdophytes (e g, ferns, horsetails, lycopods), Gymnosperms (e g, conifers, cycase, Ginko, Gnetales), and Algae (e g, Chlorophyceae, Phaeophyceae, Rhodophyceae, Myxophyceae, Xanthophyceae, and Euglenophyceae). Exemplary plants are members of the family Leguminosae (Fabaceae, e g, pea, alfalfa, soybean), Gramineae (Poaceae, e g, corn, wheat, nee), Solanaceae, particularly of the genus Lycopersicon (e g, tomato), Solarium (e g, potato, eggplant), Capsium (e g, pepper), or Nicotiana (e g, tobacco), Umbelhferae, particularly of the genus Daucus (e g, carrot), Apium (e g, celery), or Rutaceae (e g, oranges), Compositae, particularly of the genus Lactuca (e g, lettuce), Brassicaceae (Cruciferae), particularly of the genus Brassica or Sinapis In certain aspects, plants in accordance with the invention maybe species of Brassica or Arabidopsis Some exemplary Brassicaceae family members include Brassica campestns, B cannata, B juncea, B napus, B nigra, B oleraceae, B tournifortu, Sinapis alba, and Raphanus sativus Some suitable plants that are amendable to transformation and are edible as sprouted seedlings include alfalfa, mung bean, radish, wheat, mustard, spinach, carrot, beet, onion, garlic, celery, rhubarb, a leafy plant such as cabbage or lettuce, watercress or cress, herbs such as parsley, mint, or clovers, cauliflower, broccoli, soybean, lentils, edible flowers such as sunflower etc.

In advantageous embodiments, the host cell is derived from Nicotiana benthamiana, Nicotiana tabacum or derived from a tobacco BY2 cell line or Hordeum vulgare L., Zea mays or Triticum spp.

Therefore, in an advantageous embodiment, the expression vectors may be delivered to plants according to known techniques. For example, vectors themselves may be directly applied to plants (e g, via abrasive inoculations, mechanized spray inoculations, vacuum infiltration, particle bombardment, or electroporation). Alternatively or additionally, virons may be prepared (e g, from already infected plants), and may be applied to other plants according to known techniques. A wide variety of viruses are known that infect various plant species, and can be employed for polynucleotide expression according to the present disclosure (see, for example, in The Classification and Nomenclature of Viruses, “Sixth Report of the International Committee on Taxonomy of Viruses” (Ed Murphy et al), Springer Verlag New York, 1995, Grierson et al, Plant Molecular Biology, Blackie, London, pp 126-146, 1984, Gluzman er al, Communications in Molecular Biology Viral Vectors, Cold Spring Harbor Laboratory, Cold Sppng Harbor, N.Y., pp 172-189, 1988, and Mathew, Plant Viruses Online, all of which are incorporated herein by reference) In certain embodiments, rather than delivering a single viral vector to a plant cell, multiple different vectors are delivered which, together, allow for replication (and, optionally cell-to-cell and/or long distance movement) of viral vector(s) Some or all of the proteins may be encoded by the genome of transgenic plants. In certain aspects, described in further detail herein, these systems include one or more viral vector components.

The present disclosure relates also to vaccine compositions (human and/or animal vaccines) comprising an IA protein complex according to the present disclosure and a pharmaceutically acceptable carrier and/or adjuvant. In advantageous embodiments, the IA protein complexes and/or the compositions according to the present disclosure are suitable as human and/or animal vaccines against a parasite of the genus Plasmodium including Plasmodium falciparum, Plasmodium vivax, Plasmodium malariae and/or Plasmodium ovale. In an advantageous embodiment, the parasite is Plasmodium falciparum.

The disclosure pertains also to vaccine compositions comprising an IA protein complex according to the present disclosure. In order to ensure optimum performance of such a vaccine composition it is preferred that it comprises an immunologically and pharmaceutically acceptable carrier, vehicle or adjuvant. The vaccine compositions and the carrier may be in a physiologically acceptable medium.

A “vaccine” is a composition of matter comprising a formulation that, when administered to a subject, induces an immune response. Vaccines can comprise polynucleotide molecules, polypeptide molecules, and carbohydrate molecules, as well as derivatives and combinations of each, such as glycoproteins, lipoproteins, carbohydrate-protein conjugates, fusions between two or more polypeptides or polynucleotides, and the like. A vaccine may further comprise a diluent, an adjuvant, a carrier, or combinations thereof, as would be readily understood by those in the art. Due to introduced by fusion or linkage with e.g. fc receptor portion, cytokines or interleukins, Toll-like receptor ligands such that the IA protein complexes according to this disclosure themselves exhibit and provide for the adjuvant properties. In one embodiment, the vaccine composition of the present disclosure comprises therefore no further adjuvant.

An effective vaccine, wherein an IA protein complex of the disclosure is recognized by the animal, will in an animal model be able to decrease parasite load in blood and/or target organs, prolong survival times and/or diminish weight loss after challenge with e.g. a malarial parasite, compared to non-vaccinated animals.

Furthermore, the fusion protein units of the present disclosure may be coupled to a carbohydrate or a lipid moiety, e.g. a carrier, or a modified in other ways, e.g. being acetylated.

Suitable carriers are selected from the group consisting of a polymer to which the polypeptide(s) is/are bound by hydrophobic non-covalent interaction, such as a plastic, e.g. polystyrene, or a polymer to which the polypeptide(s) is/are covalently bound, such as a polysaccharide, or a polypeptide, e.g. bovine serum albumin, ovalbumin or keyhole limpet haemocyanin. Suitable vehicles are selected from the group consisting of a diluent and a suspending agent. The adjuvant is preferably selected from the group consisting of dimethyldioctadecylammonium bromide (DDA), Quil A, poly I:C, aluminium hydroxide, Freund's incomplete adjuvant, IFN-gamma, IL-2, IL-12, monophosphoryl lipid A (MPL), Treholose Dimycolate (TDM), Trehalose Dibehenate and muramyl dipeptide (MDP).

Preparation of vaccines which contain peptide sequences as active ingredients is generally well understood in the art, as exemplified by U.S. Pat. Nos. 4,608,251; 4,601,903; 4,599,231 and 4,599,230, all incorporated herein by reference.

Other methods of achieving adjuvant effect for the vaccine include use of agents such as aluminum hydroxide or phosphate (alum), synthetic polymers of sugars (Carbopol), aggregation of the protein in the vaccine by heat treatment, aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20% solution of a perfluorocarbon (Fluosol-DA) used as a block substitute may also be employed. Other possibilities involve the use of immune modulating substances such as cytokines or synthetic IFN-gamma inducers such as poly I:C in combination with the above-mentioned adjuvants.

Another possibility for achieving adjuvant effect is to employ the technique described in Gosselin et al, 1992. In brief, a relevant antigen such as an antigen of the present invention can be conjugated to an antibody (or antigen binding antibody fragment) against the Fc-receptors on monocytes/macrophages.

The vaccines are administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 micro g to 1000 micro g, such as in the range from about 1 micro g to 300 micro g, and especially in the range from about 10 micro g to 50 micro g. Suitable regimens for initial administration and booster shots are also variable but are typified by an initial administration followed by subsequent inoculations or other administrations. The manner of application may be varied widely. Any of the conventional methods for administration of a vaccine are applicable. These are believed to include oral application on a solid physiologically acceptable base or in a physiologically acceptable dispersion, parenterally, by injection or the like. The dosage of the vaccine will depend on the route of administration and will vary according to the age of the person to be vaccinated and, to a lesser degree, the size of the person to be vaccinated.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides; such suppositories may be formed from mixtures containing the active ingredient in the range of 0.5 percent to 10 percent, preferably 1-2 percent. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and advantageously contain 10-95 percent of active ingredient, preferably 25-70%.

In many instances, it will be necessary to have multiple administrations of the vaccine. Especially, vaccines can be administered to prevent an infection with malaria and/or to treat established malarial infection. When administered to prevent an infection, the vaccine is given prophylactically, before definitive clinical signs or symptoms of an infection are present.

The term “pharmaceutically acceptable carrier” includes any and all solvents, dispersion media, coatings, surfactants, antioxidants, preservatives (e.g. antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art.

A pharmaceutically acceptable carrier is preferably formulated for administration to a human, although in certain embodiments it may be desirable to use a pharmaceutically acceptable carrier that is formulated for administration to a non-human animal, such as a canine, but which would not be acceptable (e.g., due to governmental regulations) for administration to a human. Except insofar as any conventional carrier is incompatible with the active ingredient, its use in the therapeutic or pharmaceutical compositions is contemplated.

The actual dosage amount of a composition of the present disclosure administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In advantageous embodiments, the fusion proteins according to the present disclosure are used for preparing a medicament for preventing or treating malaria, in particular malaria tropica.

The present disclosure relates also to methods of producing an IA protein complex according to the present disclosure comprising the steps of:
a) culturing a host cell according to the present disclosure in a suitable culture medium under suitable conditions to produce said recombinant fusion proteins, wherein the host cell comprises all nucleic acid molecules encoding the fusion protein units of the isolated protein complex according to the present disclosure, and wherein said IA protein complex is formed in the host cell,
b) isolating said protein complex, and optionally
c) processing said protein complex.

In another embodiment, the present disclosure pertains to methods of producing a protein complex according to the present disclosure comprising the steps of:
(a) culturing of a plurality of host cells according to the present disclosure in a suitable culture medium under suitable conditions to produce said recombinant fusion proteins;
(b) isolating said produced fusion proteins,
(c) mixing said fusion proteins to produce said protein complex
(d) isolating said produced protein complex, and optionally
(e) processing the protein complex.

In some advantageous embodiments, a plurality of different HC and LC fusion polypeptides comprising different antigens were produced, wherein the produced HC and LC fusion polypeptides were mixed to obtain heteropolymeric IA protein complexes comprising a plurality of different antigens, preferably derived from different proteins presented on the surface of the pathogens, preferably in at least two or three different life cycle main stages of the pathogens.

As discussed above, in accordance with the present disclosure, the recombinant protein units and/or the whole IA protein complex may be produced in any desirable system. Vector constructs and expression systems are well known in the art and may be adapted to incorporate use of recombinant fusion polypeptides provided herein. For example, transgenic plant production is known and generation of constructs and plant production maybe adapted according to known techniques in the art. In some embodiments, transient expression systems in plants are desirable (see international patent application WO10037063A2).

In general, standard methods known in the art may be used for culturing or growing plants, plant cells, and/or plant tissues in accordance with the disclosure (e.g. clonal plants, clonal plant cells, clonal roots, clonal root lines, sprouts, sprouted seedlings, plants, etc) for production of recombinant polypeptides. A wide variety of culture media and bioreactors have been employed to culture hairy root cells, root cell lines, and plant cells (see for example Rao et al, 2002, Biotechnol Adv, 20 101).

In a certain embodiments, recombinant polypeptides in accordance with the present description may be produced by any known method. In some embodiments, a fusion protein is expressed in a plant or portion thereof. Proteins may be isolated and purified in accordance with conventional conditions and techniques known in the art. These include methods such as extraction, precipitation, chromatography, affinity chromatography, electrophoresis, and the like. The present invention involves purification and affordable scaling up of production of recombinant polypeptide(s) using any of a variety of plant expression systems known in the art and provided herein.

In some embodiments of the present disclosure, it will be desirable to isolate recombinant polypeptide(s) for the vaccine products. Where a protein in accordance with the disclosure is produced from plant tissue(s) or a portion thereof, e g, roots, root cells, plants, plant cells, that express them, methods known in the art may be used for any of partial or complete isolation from plant material. Where it is desirable to isolate the expression product from some or all of plant cells or tissues that express it, any available purification techniques maybe employed. Those of ordinary skill in the art are familiar with a wide range of fractionation and separation procedures (see, for example, Scopes et al, Protein Purification Principles and Practice, 3 rd Ed, Janson et al, 1993, Protein Purification Principles High Resolution Methods, and Applications, Wiley-VCH, 1998, Springer-Verlag, N.Y., 1993, and Roe, Protein Purification Techniques, Oxford University Press, 2001, each of which is incorporated herein by reference). Those skilled in the art will appreciate that a method of obtaining desired recombinant fusion polypeptide(s) product(s) is by extraction. Plant material (e g, roots, leaves, etc) may be extracted to remove desired products from residual biomass, thereby increasing the concentration and purity of product. Plants may be extracted in a buffered solution. For example, plant material may be transferred into an amount of ice-cold water at a ratio of one to one by weight that has been buffered with, e g, phosphate buffer. Protease inhibitors can be added as required. The plant material can be disrupted by vigorous blending or grinding while suspended in buffer solution and extracted biomass removed by filtration or centrifugation. The product earned in solution can be further purified by additional steps or converted to a dry powder by freeze-drying or precipitation. Extraction can be earned out by pressing. Plants or roots can be extracted by pressing in a press or by being crushed as they are passed through closely spaced rollers. Fluids derived from crushed plants or roots are collected and processed according to methods well known in the art. Extraction by pressing allows release of products in a more concentrated form. In some embodiments, polypeptides can be further purified by chromatographic methods including, but not limited to anion exchange chromatography (Q Column) or ultrafiltration. Polypeptides that contain His-tags can be purified using nickel-exchange chromatography according to standard methods. In some embodiments, produced proteins or polypeptides are not isolated from plant tissue but rather are provided in the context of live plants (e g, sprouted seedlings). In some embodiments, where the plant is edible, plant tissue containing expressed protein or polypeptide is provided directly for consumption. Thus, the present disclosure provides edible young plant biomass (e.g. edible sprouted seedlings) containing expressed protein or polypeptide.

Mammalian cells may be transfected by any suitable technique such as lipofection. Alternatively, standard calcium phosphate transfection or electroporation may be used, which is well understood by the skilled person. The recombinant antibodies produced from these expression systems and nucleic acid molecules of the disclosure are preferably provided in a substantially pure or homogeneous form.

Recombinant antibodies may be purified by any suitable method affinity chromatography followed using mAB select or Protein A sepharose. This may optionally be followed by a gel filtration step, e. g. using Superdex200.

To express an antibody-like recombinant fusion protein complex or antibody-like recombinant fusion protein units according to the present disclosure, a DNA encoding an immunoglobulin light chain constant domain and/or immunoglobulin heavy chain constant domain, obtained as described above, may be inserted into an expression vector such that the gene is operably linked to transcriptional and translational control sequences. In this context, the term “operably linked” means that an antibody gene is ligated into a vector such that transcriptional and translational control sequences within the vector serve their intended function of regulating the transcription and translation of the immunoglobulin gene. The expression vector and expression control sequences are chosen to be compatible with the expression host cell used. The immunoglobulin light chain constant domain gene and/or the immunoglobulin heavy chain constant domain gene can be inserted into separate vectors or, more typically, both genes are inserted into the same expression vector. The immunoglobulin genes are inserted into the expression vector by standard methods. Additionally, the recombinant expression vector can encode a signal peptide that facilitates secretion of the immunoglobulin light chain constant domain and/or immunoglobulin heavy chain constant domain from a host cell. The immunoglobulin light chain constant domain and/or immunoglobulin heavy chain constant domain gene can be cloned into the vector such that the signal peptide is operably linked in-frame to the amino terminus of the immunoglobulin chain gene. The signal peptide can be an immunoglobulin signal peptide or a heterologous signal peptide.

In addition to the antibody heavy and/or light chain gene (s), a recombinant expression vector of the invention carries regulatory sequences that control the expression of the antibody chain gene (s) in a host cell. The term “regulatory sequence” is intended to include promoters, enhancers and other expression control elements (e. g., polyadenylation signals), as needed, that control the transcription or translation of the antibody chain gene (s). The design of the expression vector, including the selection of regulatory sequences may depend on such factors as the choice of the host cell to be transformed, the level of expression of protein desired. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e. g., the adenovirus major late promoter (AdMLP)) and polyoma virus.

In addition to the antibody heavy and/or light chain genes and regulatory sequences, the recombinant expression vectors of the disclosure may carry additional sequences, such as sequences that regulate replication of the vector in host cells (e. g., origins of replication) and one or more selectable marker genes. The selectable marker gene facilitates selection of host cells into which the vector has been introduced. For example, typically the selectable marker gene confers resistance to drugs, such as G418, hygromycin, or methotrexate, on a host cell into which the vector has been introduced.

Further examples for selectable marker genes include the dihydrofolate reductase (DHFR) gene (for use in DHFR-minus host cells with methotrexate selection/amplification), the neo gene (for G418 selection), and glutamine synthetase (GS) in a GS-negative cell line (such as NSO) for selection/amplification.

For expression of the light and/or heavy chains, the expression vector (s) encoding the heavy and/or light chains is transfected into a host cell by standard techniques e. g, electroporation, calcium phosphate precipitation, DEAE-dextran transfection and the like.

Although it is theoretically possible to express the IA protein complexes or the fusion protein units comprised therein of the present disclosure in either prokaryotic or eukaryotic host cells, preferably eukaryotic cells, and most preferably mammalian host cells, because such cells, are more likely to assemble and secrete a properly folded and immunologically active antibody. Preferred mammalian host cells for expressing the recombinant antibodies of the invention include Chinese Hamster Ovary (CHO cells) (including DHFR-CHO cells 32) used with a DHFR selectable marker, e. g., as described before 33, NSO myeloma cells, COS cells, and SP2/0 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the host cell and/or the culture medium using standard purification methods. In some embodiments, the fusion protein units and/or IA protein complexes are isolated using protein A/G/L chromatography.

The disclosure pertains also to antibody compositions comprising isolated antibodies or fragments thereof which bind to an IA protein complex or to a recombinant fusion protein unit comprised therein according to the present disclosure.

According to the present disclosure, the term “antibody” includes, but is not limited to recombinant antibodies, polyclonal antibodies, monoclonal antibodies, single chain antibodies, humanized antibodies, minibodies, diabodies, tribodies as well as antibody fragments, including antigen-binding portion of the antibodies according to the present disclosure, such as Fab′, Fab, F(ab′)2 and single domain antibodies as mentioned above.

As mentioned above, the present disclosure pertains to isolated IA protein complex suitable as a vaccine comprising (i) at least two recombinant fusion proteins, (ii) at least two different antigens, (iii) at least one different homo- and/or hetero-oligomerization domain, wherein said first recombinant fusion protein comprise at least one homo- or hetero-oligomerization domain that is absent from said second recombinant fusion protein. In some advantageous embodiments, the homo- and/or hetero-oligomerization domain is selected from the group consisting of immunoglobulin domains including antibody variable domains, T-cell receptor variable and constant domains, coiled-coil domains, leucine zipper domains, collagen related triple helices, T4 bacteriophage fibritin foldon (Fd) trimerization domains, PDZ and PDZ-like domains.

### Methods and Examples

In the following example, materials and methods of the present disclosure are provided. It should be understood that these examples are for illustrative purpose only and are not to be construed as limiting this disclosure in any manner. All publications, patents, and patent applications cited herein are hereby incorporated by reference in their entirety for all purposes.

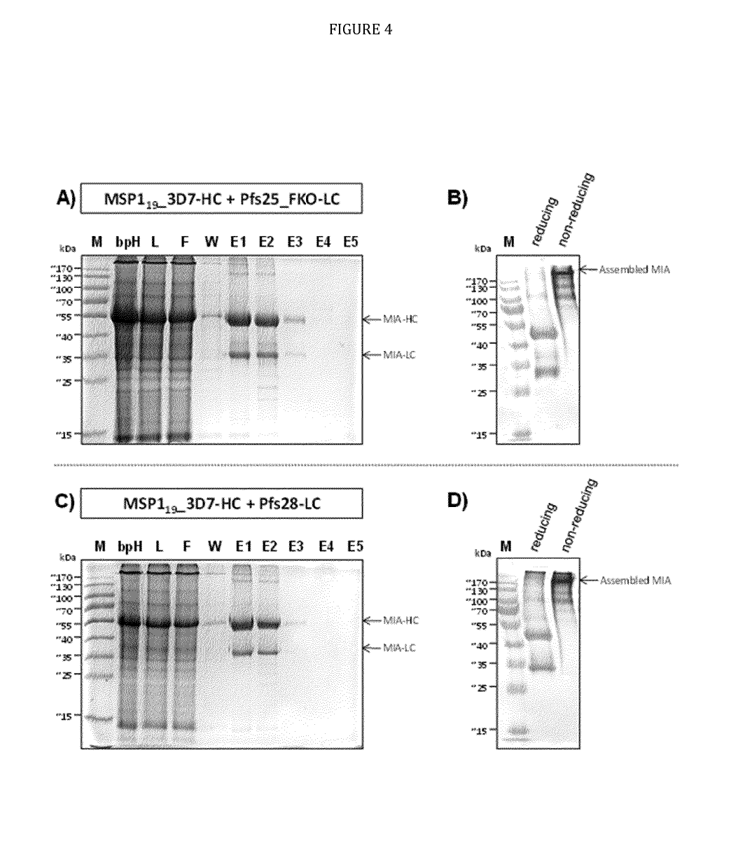

As a first example three different recombinant fusion polypeptides named PfMSP1₁₉-HC ((SEQ ID NO. 33) a fusion protein featuring PfMSP1₁₉ (SEQ ID NO. 1) and hIgG1 (SEQ ID NO. 32)), Pfs25_FKO-LC ((SEQ ID NO. 53) a fusion protein comprising leading sexual stage vaccine candidate Pfs25_FKO (SEQ ID NO. 6) genetically linked to hLCₖₐₚₚₐ (SEQ ID NO. 50)), and Pfs28-LC ((SEQ ID NO. 55) a fusion protein featuring Pfs28 (SEQ ID NO. 8) and the hLCₖₐₚₚₐ (SEQ ID NO. 50)) were coproduced in N. benthamiana plants according to 1:1 HC/LC ratios each. After purification these first proteins were used to validate their accumulation in planta as well as their assembly to antibody-like molecules incorporating at least two different plasmodial antigens from two distinct developmental stages. This first proof-of-concept is exemplary shown in FIG. 4 and FIG. 9 furthermore illustrates the combinatorial potential inherent in the herein described invention.

At a later time point and after cloning and production of several different heavy chain (SEQ ID Nos: 34-49, 67-75, 78-107) and light chain fusion polypeptides chain (SEQ ID Nos: 51-66), a specific combination (herein afterwards called ARC25) consisting of two different heavy chain and one specific light chain fusion polypeptides (ARC25-HC1.2 (SEQ ID NO. 97): a fusion protein consisting of PfAMA1_GKO (SEQ ID NO. 23) genetically linked to a modified C_{H}3 containing human IgG1 heavy chain (SEQ ID NO. 76) which is in frame with C-terminally fused PfCSP_TSR (SEQ ID NO. 9); ARC25-HC2.2 (SEQ ID NO. 88): a fusion protein containing PfRh2a₁₅ (SEQ ID NO. 25) N-terminally fused to a second modified C_{H}3 containing human IgG1 heavy chain (SEQ ID NO. 77); and the above mentioned Pfs25_FKO-LC (SEQ ID NO. 53)), covering the three main developmental stages of Plasmodium falciparum parasites: the pre-erythorcytic-, the asexual blood—as well as the sexual-stage which are illustrated in FIG. 2, were produced in N. benthamiana plants in 1:1:1 HC1.2/HC2.2/LC ratios each. After purification this specific MIA combination was lyophilized, formulated with Alum and used for the immunization of rabbits. Antibody preparations from the obtained immunesera were characterized by different methods to demonstrate the immunogenicity and the inhibitory effect on Plasmodium falciparum parasites of different stages.

As an example for an IA protein complex comprised of a fusion protein cocktail consisting of more than two non-identical HC (n=>2) fusion polypeptide units and more than two non-identical LC (n=>2) fusion polypeptide units that constitute a mixture of multiple hetero- and homopolymeric IA protein complexes (FIG. 9) based on natural immunoglobulin heavy and light chain assembly/combinatorics, three different recombinant HC fusion polypeptide units named PfAMA1_GKO-HC (SEQ ID NO. 48), MSP3A-HC (SEQ ID NO. 42) and Tetra_MSP1₁₉-HC (SEQ ID NO. 34) as well as three different recombinant LC fusion polypeptide units named PfMTRAP_TSR-LC (SEQ ID NO. 57), Pfs25_FKO-LC (SEQ ID NO. 53) and PfExp1-LC (SEQ ID NO. 63) were co-produced in leaves of a N. benthamiana plant in equimolar ratios (1:1:1:1:1:1). This mixture of three different HC and three different LC fusion polypeptide units was called “HexaMix” and after purification this cocktail was used to validate the accumulation of all six polypeptide units in planta that together incorporate six different plasmodial antigens from all three main developmental stages. Successful protein-A chromatography results based on Coomassie-stained PAA gel analysis and immunoblots utilizing antibodies specific for the incorporated malaria antigens within the six HC and LC fusion polypeptide units are exemplary shown in FIG. 16.

FIG. 16 shows a coomassie-stained poly-acrylamid gel of samples from the purification of “HexaMix” (left side) and immunoblot analysis of the individual components using the pooled and dialyzed E₁-E₆ elution fractions (right side). Equal amounts of Agrobacteria harboring expression vectors of three malaria HC fusion polypeptide units (pTRAkc-AMA1GKO-HC-ER, pTRAkc-MSP3A-HC-ER, pTRAkc-tetra_msp119-hC-ER) and three malaria LC fusion polypeptide units (pTRAkc-MTRAP_TSR-LCkappa, pTRAkc-Pfs25_FKO-LCkappa, pTRAkc-Exp1-LCkappa) were combined and the resulting suspension was infiltrated into leaves of N. benthamiana. The infiltrated leaves were harvested 5 days post infiltration and used for extraction and subsequent protein-A chromatography. Purification samples were diluted with 5×-fold reducing SDS sample buffer and electrophoretically separated (40 mA, 45 min). Immunostaining after western blotting (100 V, 60 min) was performed using the shown primary and secondary detection antibodies. M: PageRuler, L: Load (processed plant extract), F: Flow-Through, E1-6: neutralized elution fractions; Rα-: Rabbit-anti-; Mα-: Mouse-anti-; mAb 5.2: mouse-IgG specific for PfMSP1₁₉; mAb 4B7: mouse-IgG specific for Pfs25; GαM/R-AP: Goat-anti-Mouse/Rabbit-alkaline phosphatase labelled.

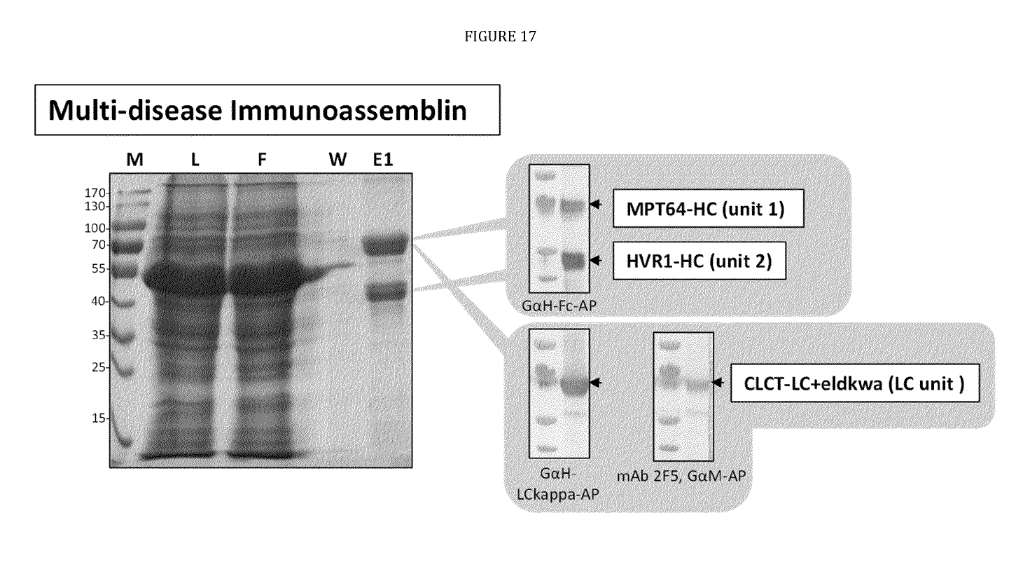

As an example for an isolated IA protein complex comprising at least three recombinant fusion protein units suitable as a vaccine against multiple diseases and not only malaria (as in the previous examples so far) a specific combination (herein afterwards called mDIA) consisting of two different heavy chains (MPT64-HC1 (SEQ ID NO. 105), HVR1-HC2 (SEQ ID NO. 106)) and one specific light chain fusion polypeptide unit (CLCT-LCedlkwa (SEQ ID NO. 107) were produced in N. benthamiana plants in 1:1:1 HC1/HC2/LC ratios each. The incorporation of several bacterial, viral and parasitic antigens enabled targeting the diseases of tuberculosis (MPT64), hepatitis c (HVR1), malaria (CLCT) and HIV (eldkwa) within and by using only one isolated IA protein complex, mDIA respectively. After 6 days post plant infiltration, infiltrated leaves were harvested and used for extraction. Recombinant mDIA protein complexes were purified by protein-A chromatography and all purification samples were analyzed by SDS-PAGE and western blotting, as illustrated in FIG. 17.

FIG. 17 shows a coomassie-stained PAA gel of Multi-disease IA purification samples and immunoblots of dialyzed elution samples E1. Agrobacteria harboring expression vectors of two HC fusion polypeptide units (pTRAkc-MPT64-HC-ER, and pTRAkc-HVR1-HC-ER) and one LC fusion polypeptide unit (pTRAkc-CLCT-LCkappa-ELDEKWA) were used to infiltrate leaves of N. benthamiana in equimolar ratios (1:1:1). 5 days post infiltration the leaves were harvested and used for extraction with subsequent protein-A chromatography. Purification samples were diluted with 5×-fold reducing SDS sample buffer and electrophoretically separated (40 mA, 45 min). Immunostaining after western blotting (100V, 60 min) was performed using the shown primary and secondary detection antibodies. M: PageRuler, L: Load (processed plant extract), F: Flow-Through, E1: neutralized and dialyzed elution fraction. MPT64: secreted protein from Mycobacterium tuberculosis; HVR1: hypervariable Region 1 of Hepatits C Virus (HCV) antigen E2; CLCT: PfCelTos-Long_Repeats+TSR of PfCSP-TRAP_{TSR}; ELDKWA: epitope of HIV1-gp41 reactive against mAb 2F5; of Rα-: Rabbit-anti-; Mα-: Mouse-anti-; mAb 5.2: mouse-IgG specific for PfMSP1₁₉; mAb 4B7: mouse-IgG specific for Pfs25; GαM/R-AP: Goat-anti-Mouse/Rabbit-alkaline phosphatase labelled.

1. Cloning of Expression Constructs

The antigen fragment sequences listed in Table 1 (SEQ ID NOS. 1-31) were optimized for plant expression (GeneArt). To generate MIA starting vectors for human HC and LC fusion polypeptides incorporating malaria antigens like schematically shown in FIG. 3, the genetic information of the human IgG1 constant domain and the human light chain kappa constant domain were amplified using the shuttle vector pUC57-HCgamma and pUC57-LCkappa as a PCR template. The amplified sequences were inserted into the plant expression vector pTRAkc-MSP1₁₉_{_}3D7-ERH as NotI and BamHI fragments, resulting in plant expression vectors designated as pTRAkc-MSP1₁₉_{_}3D7-HC_hIgG1-ER and pTRAkc-MSP1₁₉_{_}3D7-hLCkappa. To N-terminally exchange the malaria antigens/ligands/functional component ORFs, the latter constructed plant expression MIA starting vectors (pTRAkc-MSP1₁₉_{_}3D7-HC_hIgG1-ER and pTRAkc-MSP1₁₉_{_}3D7-hLCkappa, respectively) containing the mentioned MSP1₁₉_{_}3D7 gene were treated by NcoI and NotI to remove this placeholder malaria antigen and to insert all following antigens/ligands/functional component ORFs as NcoI and NotI digested fragments. All heavy chain fusion polypeptide constructs generated in this way carried a C-terminal SEKDEL-tag for ER retrieval (Pelham, 1990). Due to theoretical in vivo and in planta assembly of heavy and light chain fusion polypeptides, the latter described ones were not additionally equipped with said C-terminal tags. A detailed description of the pTRAkc plasmid is reported in Boes et al (Boes et al., 2011).

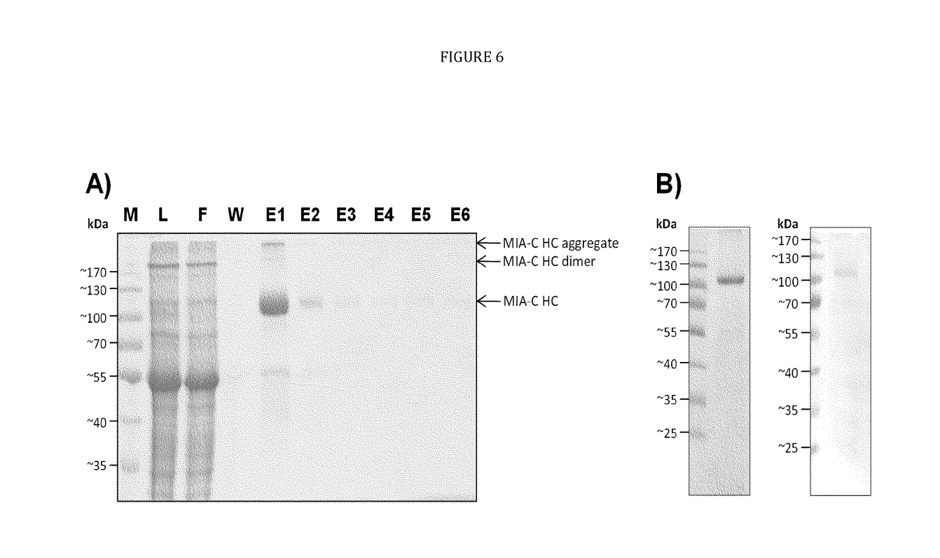

To enable further C-terminal additions of malaria antigens/ligands/functional components to heavy chain polypeptide fusions like depicted in FIG. 5, in a first step the vector of pTRAkc-MSP1₁₉-ERH was used as a PCR template to amplify the MSP1₁₉_{_}3D7 (SEQ ID NO. 1) ORF and to equip it N-terminally with a PciI restriction site. Analogous, in a second step the plasmid containing the genetic information of Tetra_MSP1₁₉-HC (SEQ ID NO. 34) was enriched by PCR and C-terminally provided with a PciI recognition sequence. One has to know that digestions performed with PciI result in NcoI compatible ends. The first mentioned amplicon was treated with PciI/NotI, the second PCR product was NcoI/PciI digested and both cleaved fragments were inserted into a NcoI/NotI processed pTRAkc plasmid, thus gaining the MIA-C starting vector pTRAkc-Tetra_MSP1₁₉-HC-PciI-MSP1₁₉_{_}3 D7-ERH, afterwards herein called “PciI vector”. To C-terminally exchange the malaria antigens/ligands/functional component ORFs, the latter constructed PciI vector further underwent PciI/NotI treatment to ensure removal of the placeholder malaria antigen gene of MSP1₁₉_{_}3D7 (SEQ ID NO. 1) and to insert all following antigens/ligands/functional component ORFs as NcoI and NotI digested fragments. FIG. 6 A+B described antigen repertoire extension by genetically C-terminal addition is exemplary shown for dual-stage covering candidate Tetra_MSP1₁₉-HC-CSP_{TSR}-ERH. Accessibility of all included malaria antigens was verified by ELISA and SPR using specific antibodies.

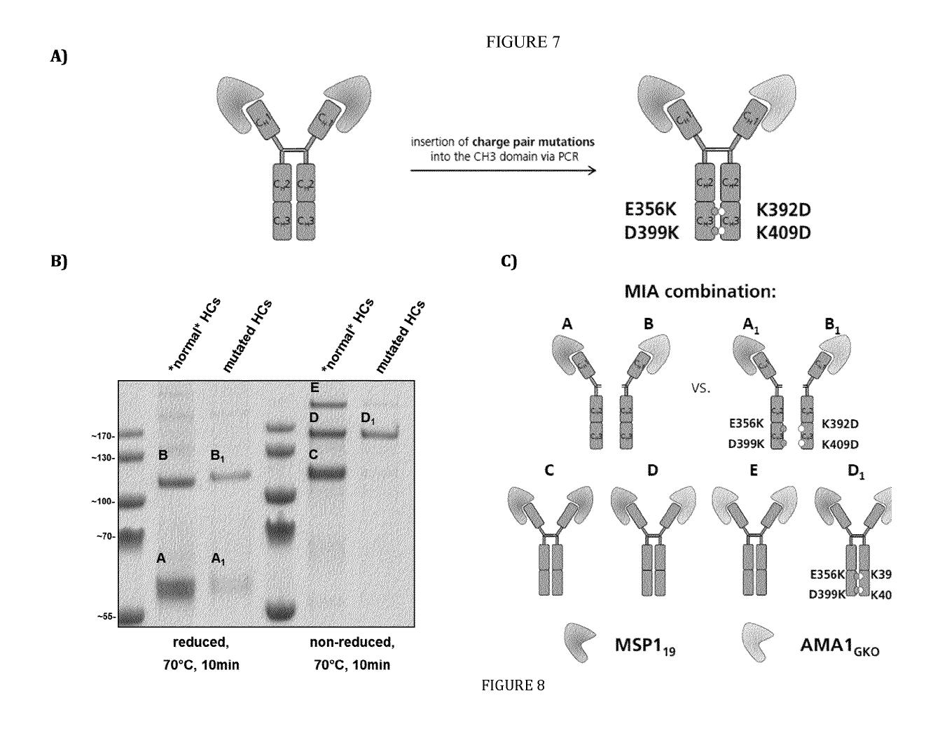

For further enhancement of the combinatorial capacity/potential and to enable almost exclusive heterodimer formation of two different (malaria) heavy chain fusion polypeptides like schematically shown in FIG. 7A while disfavoring homodimerization due to electrostatic steering effects, expression vectors of MSP1₁₉_{_}3D7-HC (SEQ ID NO. 33) and AMA1_GKO-HC (SEQ ID NO. 48) were genetically modified by PCR site-directed mutagenesis, each at two specific amino acid positions in the CH3 part of the human IgG1. Negative charge residue pairs of E356 and D399 on MSP1₁₉_{_}3D7-HC (SEQ ID NO. 33) were changed to positively charged Lys resulting in MSP1₁₉_{_}3D7-HC1.2-ER (SEQ ID NO. 78). The positively charged residues at positions K392 and K409 of the AMA1_GKO-HC (SEQ ID NO. 48) were analogously switched to negatively charged Asp, generating the expression vector AMA1_GKO-HC2.2 (SEQ ID NO. 85). The denotation of the applied amino acid replacements features wild type residues followed by the position using the Kabat (Kabat et al., 1991)/crystal structure numbering system and the mutated residue in single letter code. To validate applied changes and to assess the formation of homo- and heterodimer yield, SDS polyacrylamide gel electrophoresis was performed and results meeting the expectations are shown in FIG. 7 B+C. To generate further MIA and MIA-C heavy chain polypeptides including above mentioned charge pair modifications, expression vectors MSP1₁₉_{_}3D7-HC1.2-ER (SEQ ID NO. 78) and AMA1_GKO-HC2.2 (SEQ ID NO. 85) as well as plasmids of selected malaria antigen HC fusion polypeptides were KasI/NsiI (single cutters within the heavy chain constant domain) treated. Charge pair mutations bearing IgG1 constant domains were exchanged against the unmodified originals, resulting in a large set of MIA (SEQ ID NOS. 76-92) and MIA-C(SEQ ID NOS. 93-104) constructs with modified CH3 regions for Fc heterodimerization.

All recombinant gene constructs were verified by sequencing and introduced into Agrobacterium tumefaciens strain GV3101 (pMP90RK) by electroporation. The recombinant Agrobacterium tumefaciens were cultivated as described previously (Vaquero et al., 1999, Sack et al., 2007). The optical density (OD) of the cultures was determined and expression strains were mixed with the agrobacterium strain carrying the silencing suppressor p19 (Plant Bioscience Limited, Norwich, England) at a 5:1 ratio to a final OD of 1.

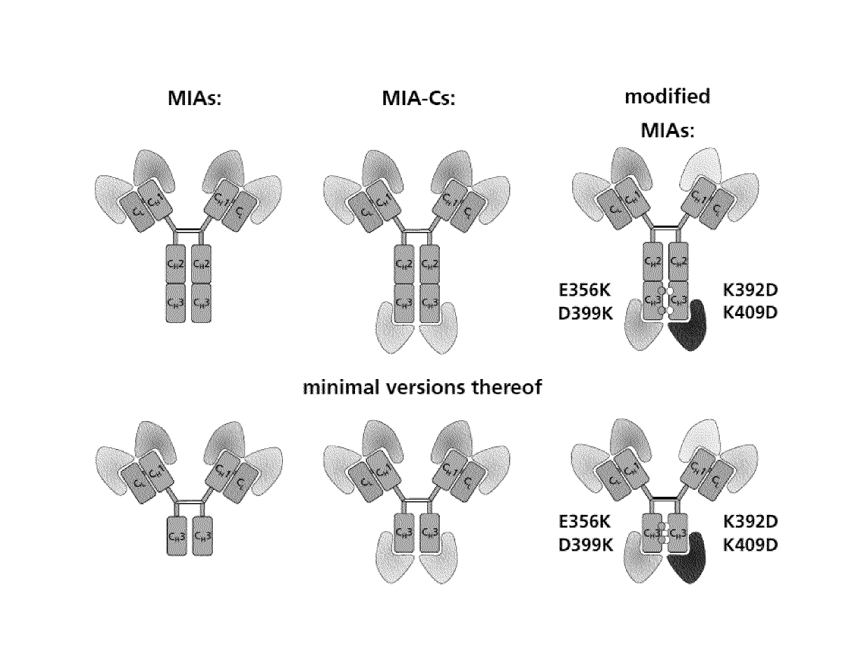

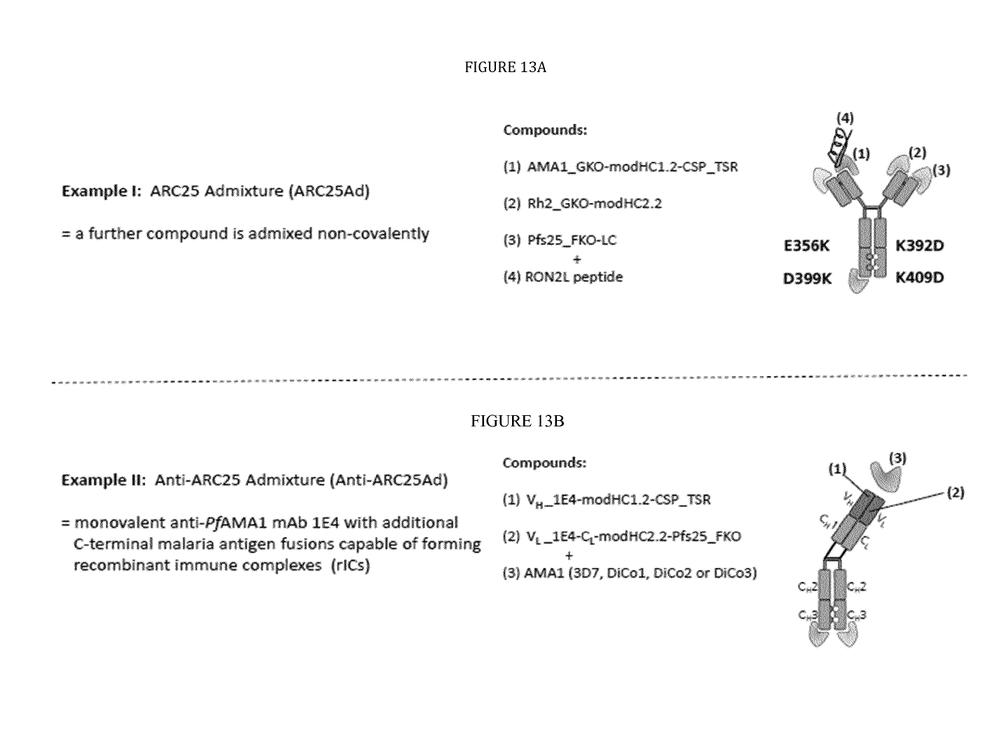

FIG. 8 shows a schematic overview of the different MIA construct format and FIG. 13 A+B depicts optimized and extended, ARC25-based MIA concepts desired for human vaccination:

2. Transient Expression

For each construct the recombinant bacteria containing the respective expression cassette were separately cultured. Before manual or vacuum injection into 6-8 week old Nicotiana benthamiana plants grown in rockwool, expression strains carrying (malaria) heavy chain and light chain fusion polypeptides were mixed at a 1:1(:1) ratios to a final OD of 1 (silencing suppressor p19 being provided at a ratio of 5:1 of the total infiltration medium). Infiltrated Nicotiana benthamiana plants were incubated for 5 days at 22° C. with a 16 h photoperiod. Plant leaf tissue was harvested for protein extraction and purification.

3. Protein Extraction

Leaf tissue was ground in liquid nitrogen using mortal and pestle and soluble proteins were extracted with 2 ml extraction buffer (PBS pH 7.4 supplied with 10 mM sodium disulfide) per gram of leaf material. Insoluble material was removed by centrifugation (16,000×g, 20 min, 4° C.). Additional salt- (PBS with 500 mM NaCl pH 7.4) and pH-shift (up to pH 8.0) steps to efficiently remove further plant host cell components were performed for all malaria HC and LC fusion polypeptides. Afterwards insoluble material was removed by a second centrifugation (16,000×g, 20 min, 4° C.) and a series of subsequent filtration steps (Miracloth, Merck Darmstadt, Germany; Whatman Klariflex 0.22 μm PES filter unit, GE Healthcare, Germany).

4. Protein a Purification

All protein combinations of interest were purified using protein A chromatography (GE Healthcare, Germany). The target proteins were captured on chelating sepharose charged with protein A. After a washing step with PBS including 500 mM NaCl, adjusted to pH 8.0, the target protein was eluted using 100 mM glycine dissolved in ddH₂O at pH 3.0. Subsequent neutralization was achieved with 1/10 elution volume of 1M TRIS pH 8.8.

5. Immobilized Metal Ion Chromatography (IMAC)

Single malaria antigens/ligands/functional polypeptide components ((SEQ ID NOS: 1-31) which were used to generate the different MIA constructs) featuring a C-terminal hexa-histidine and a C-terminal SEKDEL-tag for ER retrieval tag were purified by immobilized metal ion chromatography (IMAC). After the pH of the extract was adjusted to pH 8.0 and NaCl was added to a final concentration of 500 mM, the target proteins were captured on chelating sepharose charged with nickel. After a washing step with PBS adjusted to pH 8.0 the target proteins were eluted in a step gradient at 15 mM, 50 mM and 250 mM imidazole dissolved in PBS at pH 8.0.

6. Immunization of Rabbits

The purified polypeptide mixture ARC25 (SEQ ID NOS. 97, 88, 53) was introduced into 1.150 μL saline to prepare a solution containing 200 μg/ml which was then directly freeze-dried applying 0.310 mbar negative pressure for 72 h with an ALPHA 1-4 LSC device (Christ, Osterode am Harz, Germany). The lyophilized ARC25 samples were sent to Biogenes (Berlin, Germany) and used for immunization of rabbits. Prior to animal vaccination, this lyophilized mix was thawed, further diluted with 1.150 μL of the adjuvant Alhydrogel®2% (Brenntag Biosector, Frederikssund, Denmark) resulting in a relevant ARC25 concentration of 100 μg/mL. This formulation was used to immunize rabbits on days 0, 28 and 56 using 500 μL of the formulated ARC25 (yielding a dose of 50 μg ARC25/immunization step). On day 70, two weeks after the last boost, final bleed sera of the vaccinated animals were obtained and used for subsequent experiments.

In addition to ARC25 a control, herein afterwards named BSSC, featuring the same amounts of AMA1_GKO (SEQ ID No. 23) and Pfs25_FKO (SEQ ID No. 6), being included in the ARC25 vaccine composition, were prepared and used analogously to ARC25 as mentioned above. [g1]

7. Protein A Purification of Antibodies from Rabbit Sera

After immunization the antibodies from the rabbit antisera were purified by protein A chromatography. Briefly, serum samples were diluted 1:5 with PBS and filtered through 0.45 μm filter prior purification. The antibodies were bound onto protein A resin (GE Healthcare) and unbound impurities were removed by a washing step with PBS pH 7.4. The bound antibodies were eluted with 100 mM glycine pH 3.0 and directly neutralized with 1/10 elution volume of 1M TRIS pH 8.8. A buffer exchange against RPMI1640 containing 25 mM HEPES and no L-Glutamine (E15-041, PAA) was performed using a HiPrep Desalting column and the antibodies were concentrated by centrifugal concentration devices (VivaSpin 15R 30.000 MWCO, Sartorius) to a concentration greater than 12 mg/ml and sterile filtered. Antibodies were stored at 4° C.

8. SDS-PAGE and Immunoblot Analysis

Proteins were separated on self-poured SDS (8%, 12% or 15%) or commercial 4-12% (w/v) gradient gels (Invitrogen) under reducing and/or non-reducing conditions and stained with Coomassie R-250 following the Fairbanks protocol (Wong et al. 2000). Separated proteins were blotted onto a nitrocellulose membrane (Whatman, Dassel, Germany) and blocked with 5% (w/v) skimmed milk dissolved in PBS. MIAs were probed with detection antibodies Goat-anti-Human-Fc and Goat-anti-Human-LCkappa, both labelled with alkaline phosphatase. Single malaria antigens/ligands/functional polypeptide components desired for titer determination were probed with Rabbit anti-His6-tag as primary antibody at a 1:5.000 dilution. In this case the applied secondary antibody was Goat anti-Rabbit H+L alkaline phosphatase labeled. Bands were visualized with NBT/BCIP (1 mg/mL in substrate buffer: 150 mM NaCl, 2 mM MgCl2, 50 mM Tris-HCl, pH 9.6). Between the incubation steps the membranes were washed three times with PBS supplemented with 0.05% (v/v) Tween-20.

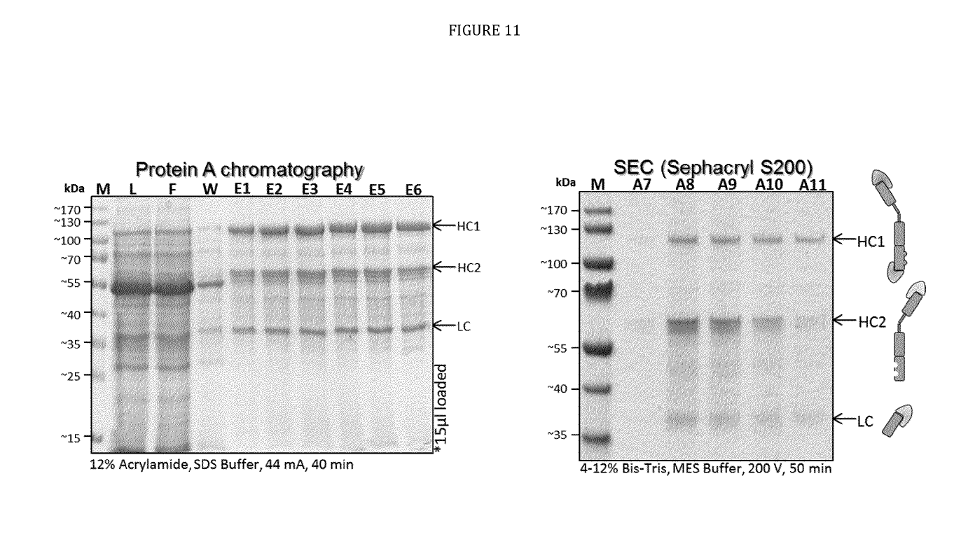

FIG. 10: SDS-PAGE analysis of the purified recombinant protein ARC25 according to the present example. ARC25 was separated under reducing conditions. FIG. 10 shows a schematic depiction of ARC25 and included components covering main developmental stages of Plasmodium falciparum. FIG. 11 shows Coomassie stained and destained SDS-PAGE gels of ARC25, first after protein A and second post size exclusion chromatography (SEC). Molecular weight standard is indicated at the left site.

The abbreviations in FIG. 11 are:
M: PageRuler Prestained Protein Ladder (Thermo Scientific)
L: Load (processed extract)
F: Flow-through
W: Wash (1×PBS, pH 7.4)
E1-E6: Protein A elution fractions containing target protein(s)
A7-A11: SEC elution fractions containing target protein(s)
HCl: ARC25-HC1.2 (SEQ ID NO. 97)
HC2: ARC25-HC2.2 (SEQ ID NO. 88)
LC: Pfs25_FKO-LC (SEQ ID NO. 53)

9. ELISA

The specific antibody (IgG) titer in the serum against the protein ARC25 (SEQ ID NOS. 97, 88, 53) used for immunization as well as the reactivity against all single malaria antigens/ligands/functional polypeptide components was measured by ELISA using high-binding 96 well plates (Greiner bio-one, Frickenhausen, Germany) coated with recombinant proteins at a concentration of 100 ng/well. After 1 h incubation at room temperature. the wells were blocked with 5% (w/v) skimmed milk in PBS and incubated again for 1 h at room temperature. A serial dilution of the sera and the pre-immune serum (as negative control and for blank subtraction) were applied to the 96 well plate and incubated for 1 h at room temperature. The antigen-bound antibodies were probed with HRPO-labeled Goat anti-Rabbit IgG Fc and detected with ABTS substrate at 405 nm after 30 min. Between each step, the plates were washed three times with PBS supplemented with 0.05% (v/v) Tween-20. The specific IgG titer was defined as the dilution which results in an OD 405 nm 1,5-fold the value of the pre-immune serum.

TABLE 7
Rabbit antibody titers raised against ARC25 (SEQ ID NOS. 97, 88, 53 listed in Table 1)

| | | Minimal balanced antibody titer against every antigen | | |
|---|---|---|---|---|
| | | fragment included in vaccine candidate (SEQ IDs NO. 97, 88, 53) | | |
| Malaria antigen fragments | | Rabbit | Rabbit | Rabbit |
| included in vaccine candidate | Assay | 24700 | 24701 | 24702 |
| ARC25 (SEQ ID NOS. 97, 88, 53 | ELISA | 1:220.000 | 1:280.000 | 1:274.000 |
| pre-erythrocytic stage: | | | | |
| PfCSP_TSR (SEQ ID NO. 9) | | 1:32.000 | 1:48.000 | 1:24.000 |
| asexual/blood stage: | | | | |
| PfAMA1_GKO (SEQ ID NO. 23) | | 1:32.000 | 1:64.000 | 1:32.000 |
| PfRh2₁₅_{_{—}}GKO (SEQ ID NO. 25) | | 1:12.000 | 1:24.000 | 1:12.000 |
| sexual stage: | | | | |
| Pfs25_FKO (SEQ ID NO. 6) | | 1:128.000 | 1:128.000 | 1:190.000 |

10. Immunofluorescence-Assay (IFA)

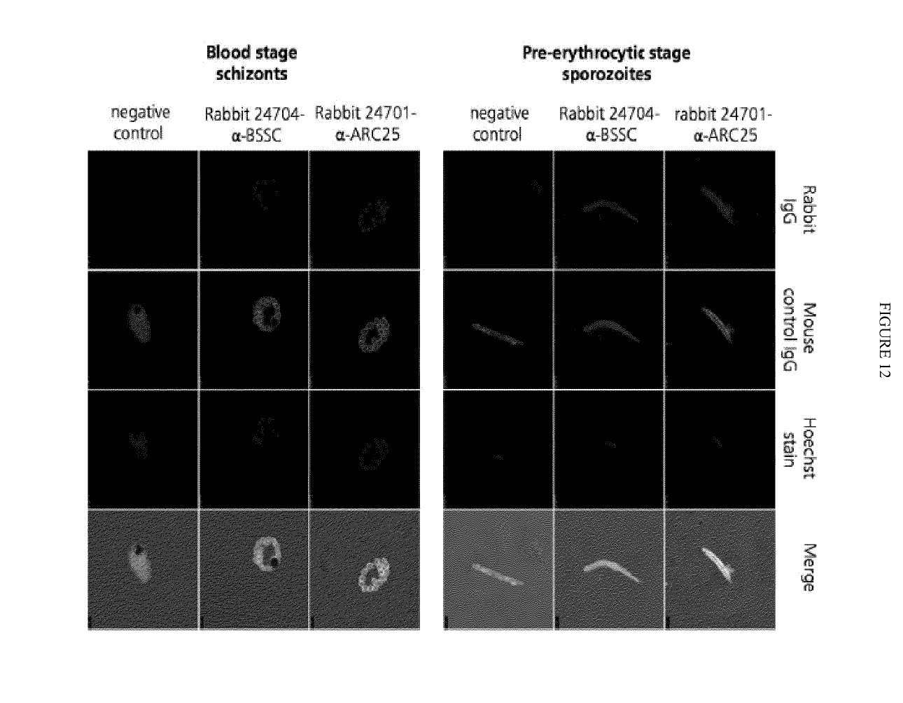

To visualize the different developmental stages of the P. falciparum parasites, indirect IFAs were performed as described previously (Pradel et al, 2004). Cultivation of asexual stages and gametocytes of P. falciparum strain NF54 were performed as described previously (Ifediba and Vanderberg, 1981). Parasite preparations were air dried on 8-well diagnostic slides (Thermo scientific) and fixed with −80° C. methanol for 10 min. To block nonspecific binding and to permeabilize membranes, fixed cells were incubated in 0.5% BSA, 0.01% saponin in PBS for 30 min at RT and subsequently in 0.5% BSA, 0.01% saponin, 1% neutral goat serum in PBS for 30 min at RT. Samples were incubated with the purified antibodies directed against ARC25, diluted in blocking solution without goat serum at 37° C. for 1 h. Purified antibodies were used at a final concentration of 50 μg/ml. For counterstaining of the different P. falciparum life cycle stages, mouse antisera directed against single P. falciparum antigen fragments from PfCSP_TSR (counterstaining of sporozoites), MSP1₁₉ (counterstaining of schizonts) or Pfs25 (counterstaining of macrogametes and zygotes) were generated by Fraunhofer IME and used in final concentrations of 20 μg/mL. Primary antibodies were visualized by incubation of cells with fluorescence-conjugated Alexa Fluor 488 goat-anti-mouse (Invitrogen #A-11001; 1:100) or Alexa Fluor 594 (JIR 111-585-144; 1:200) goat-anti-rabbit antibodies in blocking solution without goat serum. To highlight nuclei, samples were incubated with Hoechst 33342 (10 μg/mL) in PBS supplied with 0.01% saponin. Finally, cells were mounted with anti-fading solution ProLong® Gold Antifade Mountant (Invitrogen # P36934) and sealed with nail varnish. Examination of labeled cells and scanning of images was performed using a Leica TCS-SP8 spectral confocal microscope. Exemplary immunofluorescence assays of different Plasmodium falciparum stages with purified rabbit antibodies raised against ARC25 according to the present disclosure are illustrated in FIG. 12. In each section of the Figure staining with purified rabbit pAb raised against ARC25 (detection with anti-rabbit pAb labeled with Alexa594) are shown on the left, a positive control staining in the second left (murine control pAb, detection with anti-mouse pAb labeled with Alexa488) and a Hoechst nuclear staining is shown on the third left. On the very right merged overlay images are shown. Negative controls (performed using the pre-immune rabbit sera) are depicted in the last row.

FIG. 12: Immunolabelling of the pre-erythrocytic and the asexual blood stage of P. falciparum with rabbit immune sera generated against ARC25 and BSSC. IFAs were performed on methanol-fixed sporozoites and blood-stage schizonts using the protein A-purified rabbit IgGs from rabbit serum samples collected on day 70 after immunization with either ARC25 or BSSC. Exemplary shown are two rabbit IgG samples (rabbit no. 24701 for ARC25) rabbit no. 24704) immunolabeled the whole surface of sporozoites as well as the apical pole of merozoites enclosed by the schizonts (in red). Mouse-anti-PfCSP_TSR monoclonal antibody 6.75M was applied to detect the sporozoite surface and mouse anti-PfMSP1₁₉ antiserum was used to counterstain the merozoite plasmalemma (in green); the parasite nuclei were highlighted with Hoechst 33342 (in blue). Purified IgG-fractions of immunized rabbits were used at a concentration of 15 mg/mL and were evaluated for binding to the native P. falciparum surface (in red).

11. Inhibition of Sporozoites Gliding Motility (SGM), Hepatocyte Cell Traversal (HCT) and Sporozoite Invasion and Liver Stage Development (SILSD)

Gliding motility and cell traversal assays were carried out to test the effect of purified IgG fractions from immunized rabbits on the gliding ability and the hepatocyte traversal capacity of P. falciparum sporozoites. Both assays were performed as described by Behet et al. (2014), with minor modifications. For the gliding motility assay 96-well glass bottom black plates pre-coated with 30 μg anti-CSP mAb 3SP2 were used to capture shed CSP protein. A number of 10,000 P. falciparum sporozoites were pre-incubated in triplicates with 10 mg of rabbit IgG (anti-ARC25: pooled ARC25 samples; ant-BSSC: pooled for 30 min on ice and then transferred onto the 3SP2 coated slides. After 90 min incubation at 37° C. in 5% CO2 sporozoites were washed off. Gliding trails were fixed with 4% paraformaldehyde (PFA) for 20 min at RT and stained with anti-CSP-biotin followed by streptavidin-AF594. Gliding trails were visualized by fluorescent microscopy at 1000× magnification and images were analyzed with FIJI imaging software.

For the HCT, hepatocyte cell line HC-04 cells were seeded in a 96-well clear bottom black plate and grown until confluency. Per well, 50,000 P. falciparum sporozoites were pre-incubated with 10 mg of rabbit IgG for 30 min and then overlayed onto the hepatocytes in the presence of rhodamine-dextran. After 3 h incubation, sporozoites were washed off. Uptake of rhodamine-dextran as a result of sporozoite traversal was quantified by measuring the fluorescence signal in a plate reader (excitation: 540/35 nm, emission: 600/40 nm).

The SILSD assay was performed according to van Schaijk et al. (2008), to test the effect of purified and pooled IgG fractions from immunized rabbits on the capacity of P. falciparum sporozoites to infect human primary hepatocytes. Minor adaptions were carried out, as 50,000 cryopreserved human primary hepatocytes were seeded in a 96-well clear bottom black plate and refreshed daily for 2 days. Salivary gland sporozoites were isolated from mosquitoes infected with P. falciparum NF54. Per well 50,000 sporozoites were pre-incubated with IgG for 30 min on ice and then transferred onto the hepatocytes. After 3 h, non-invaded sporozoites were washed off and hepatocytes were refreshed daily for 6 days. Cells were washed, fixed and stained intracellular. Vehicle and 3SP2 samples were stained with DAPI and rabbit anti-PfHSP70. Vehicle and rabbit IgG samples were stained with DAPI and a pool of mouse mAbs directed against PfBip (Binding protein, ER marker), PfExp1 (Exported protein 1), PfHSP70 (Heat Shock Protein 70) and PfMSP1 (merozoite specific protein 1). The number of positively-stained infected hepatocytes was determined by automated microscopy in 25 fields at 100× magnification using FIJI image analysis software. The SGM, HCT and SILSD assay results of purified rabbit antibodies raised against ARC25 according to the present disclosure are listed below in Table 8.

12. Growth Inhibition Assay (GIA)

The growth inhibitory potential against Plasmodium parasites was performed using a standardized protocol. The P. falciparum parasite strain 3D7A (provided by MR4) was maintained in culture at parasitemias below 5% at a haematocrit of 4% in RPMI medium supplemented with 10% Albumax II (Invitrogen), 25 mM Hepes, 12 μg/ml gentamicin and 100 μM hypoxanthine at 37° C. and 5% CO₂, 5% O₂ and 90% N₂. The cultures were maintained in a daily routine and parasitemia was estimated by Giemsa staining. The erythrocytes used in the assay were mixed from 15 malaria-naïve blood donors and not older than 3 weeks. The erythrocytes were stored in SAG-Mannitol at 4° C. The parasites were synchronized by 10% Sorbitol treatment within a time window of 1-16 hours post invasion. For the assay, only highly synchronous cultures 36 to 40 hours post invasion were used.

Parasites, fresh RBCs and antibodies were mixed in a 96-well plate appropriately in order to have a final parasitemia of 0.1% and a final haematocrit of 2%. For the background control, only RBCs without parasites were kept in culture under the same conditions as the parasites. A growth control for monitoring the parasite growth was performed by culturing the Plasmodium falciparum parasites without additions. All samples were measured in triplicates. As negative control, malaria-naïve rabbit and human plasma were derived and purified antibodies were tested. For positive control of complete invasion inhibition, EDTA (4 mM final concentration) and BG98 rabbit anti-AMA-1 polyclonal antibodies were used. The plates were incubated at 37° C., 95% humidity, 5% CO2, 5% O2, and 90% N2 for 40 to 44 hours. At harvest, wells were washed once with cold PBS and frozen down. Parasite growth was estimated by a Malstat™ assay32. Absorbance was measured after 30 minutes at a wavelength of 655 nm using a spectrophotometer. Inhibitory capacity was estimated by the following formula:

% inhibition=100%−((A655 IgG sample−A655 RBC control))/((A655 Schizont control−A655 RBC control))*100%

As mentioned above, the growth inhibition assay is a standard in vitro assay to evaluate the inhibitory potential of antibodies. The assay simulates the asexual stage/blood stage. The GIA results of purified rabbit antibodies raised against ARC25 and BSSC are listed in Table 5.

13. Luminescent Standard Membrane Feeding Assay (TropIQ Health Science, Nijmegen, Netherlands)

To assess the effect of purified IgG fractions from immunized rabbits on the capacity of P. falciparum stage V gametocytes to infect Anopheles stephensi mosquitoes, membrane feeding assays were performed (Bishop and Gilchrist, 1946). Briefly, test samples were combined with stage V gametocytes from P. falciparum strain NF54-hsp70-luc, human red blood cells and fed to Anopheles stephensi mosquitoes. The experiment was performed in presence of active complement. After 8 days, luciferase expression in individual mosquitoes was analyzed.
Single dose per sample dilution, analyses of luminescence intensity in 24 mosquitoes per sample/feeder (N=1, n=24)
Analyses of 24 uninfected mosquitoes to determine luminescence background levels
Controls in duplicate: PBS vehicle and transmission-blocking mAb 85RF45.1 at IC90

Data Analyses and Reporting
Data are expressed as luminescence intensities (counts per second (cps))
Data reported include: number of analyzed mosquitoes, number of oocysts per mosquito in vehicle controls, oocyst intensity and oocyst prevalence, raw data

Assay Quality Criteria
70% oocyst prevalence in at least one of the negative control feeders
At least 50% of the feeders has 20 observations (mosquitoes) with a minimum of 15 observations for every feeder
At least 75% inhibition of luciferase signal by positive control mAb 85RF45.1 at IC90

TABLE 8
Results of SMFA on stage V gametocytes
Control samples

| | | | Oocyst prevalence | | |
|---|---|---|---|---|---|
| Oocyst intensity (cps) | | | (number of positive mosquitoes/ | | |
| | Final | | total number of mosquitoes) | | |
| Sample ID | concentration | Feeder 1 | Feeder 2 | Feeder 1 | Feeder 2 |
| PBS | — | 4055 ± 2881 | 6889 ± 2657 | 16/16 | 24/24 |
| 85RF45.1 | 5 μg/ml | 303 ± 392 | 282 ± 328 | 19/24 | 20/24 |
| Negative | — | 7 ± 2 | — | — | — |
| mosquitoes | | | | | |

| | | | Oocyst prevalence |
|---|---|---|---|
| | | | (number of positive |
| | | | mosquitoes/total |
| | Final | Oocyst intensity | number of |
| Sample ID | Concentration | (cps) | mosquitoes) |
| NRS | 0.50 mg/ml | 8498 ± 5063 | 23/24 |
| ARC25 | 0.50 mg/ml | 660 ± 616 | 23/24 |
| BSSC | 0.50 mg/ml | 604 ± 751 | 23/24 |

Mosquitoes that fed on two PBS feeders showed an average of 42.4 oocysts per mosquito and this corresponded to −5755 luminescence counts per second. Mosquitoes that fed on a feeder with 5 μg/ml mAb 85RF45.1 showed on average ˜292 luminescence counts per second.

Mosquitoes that fed from a blood meal containing different concentrations of NRS showed a dose-dependent reduction of luminescence counts with increasing NRS concentrations, but all of these were comparable to the PBS control.

Mosquitoes that fed from a blood meal containing either 0.5 mg/ml ARC25 (pool of rabbits #24700-#24702) or 0.5 mg/ml BSSC (#24703-#24705) showed a significant inhibition of 92% and 93% respectively compared to NRS at 0.5 mg/ml.

The SMFA results on stage V gametocytes of purified rabbit antibodies raised against ARC25 and BSSC are included and listed in summarizing Table 9.

TABLE 9
Exemplary inhibition results of purified rabbit antibodies derived against
the components of ARC25 according to the present disclosure.

| Pathogen stage | Inhibition assay | Inhibition [%] |
|---|---|---|
| pre-erythrocytic stage | SGM | 22 |
| ARC25-HC1.2 (SEQ ID NO. 97) | HCT | 0 |
| | SILSD | 26 |
| asexual/blood stage | | |
| ARC25-HC1.2 (SEQ ID NO. 97) | growth | >40 |
| | inhibition assay | |
| ARC25-HC2.2 (SEQ ID NO. 88) | | |
| sexual stage | | |
| ARC25-LC (SEQ ID NO. 53) | SMFA on stage | 92 |
| | V gametocytes | |

The results demonstrate the feasibility to produce exemplary antigens according to the present disclosure based on Plasmodium falciparum surface proteins or protein domains of three Plasmodium life cycle stages. The production was accomplished in Nicotiana benthamiana plants. After purification the recombinant protein ARC25 elicited a balanced antibody response in animals with a titer greater than 2×10⁴. Immune fluorescence assays confirmed that the induced antibodies specifically bind to the native Plasmodium antigens. Further, functional assays demonstrated specific parasite inhibition in every corresponding Plasmodium life cycle stage in a range from 30-100%.

### REFERENCES

The contents of all cited references, including literature references, issued patents, and published patent applications, as cited throughout this application are hereby expressly incorporated by reference.
Altschul, S. F., Gish, W., Miller, W., Myers, E. W. & Lipman, D. J. Basic local alignment search tool. Journal of molecular biology 215, 403-10 (1990).
Ausubel, F. M. et al. Current protocols in molecular biology, edited by M. Ausubel, R. Brent, R. E. Kingston, D. D. Moore, J. G. Seidman, J. A. Smith, and K. Struhl. Volumes 1 and 2. John Wiley & Sons, Inc., Media, P A, 1988, 165.00. Molecular Reproduction and Development 1, 146-146 (1989).
Behet, M. C. et al., (2014). “Sporozoite immunization of human volunteers under chemoprophylaxis induces functional antibodies against pre-erythrocytic stages of Plasmodium falciparum.” Malar J 13: 136.
Beier M S, Pumpuni C B, Beier J C, Davis J R. 1994. Effects of para-aminobenzoic acid, insulin, and gentamicin on Plasmodium falciparum development in anopheline mosquitoes (Diptera: Culicidae). J. Med. Entomol. 31(4): 561-565
Bergmann-Leitner E S, Mease R M, De La Vega P, Savranskaya T, Polhemus M, Ockenhouse C, Angov E. 2010. Immunization with pre-erythrocytic antigen CelTOS from Plasmodium falciparum elicits cross-species protection against heterologous challenge with Plasmodium berghei. PLoS One 5(8):e12294.
Bishop A and Gilchrist B M. 1946. Experiments upon the feeding of Aedes aegypti through animal membranes with a view to applying the method to the chemotherapy of malaria. Parasitology. 37: 85-100
Black C G, Wang L, Wu T, Coppel R L. 2003. Apical location of a novel EGF-like domain-containing protein of Plasmodium falciparum. Mol Biochem Parasitol 127(1):59-68.
Black C G, Wu T, Wang L, Hibbs A R, Coppel R L. 2001. Merozoite surface protein 8 of Plasmodium falciparum contains two epidermal growth factor-like domains. Mol Biochem Parasitol 114(2):217-26.
Black, C. G., Wang, L., Wu, T. & Coppel, R. L. Apical location of a novel EGF-like domain-containing protein of Plasmodium falciparum. Molecular and biochemical parasitology 127, 59-68 (2003).
Black C G, Wu T, Wang L, Hibbs A R, Coppel R L. Merozoite surface protein 8 of Plasmodium falciparum contains two epidermal growth factor-like domains. Mol Biochem Parasitol 2001; 114:217-26.
Blackman M J, Ling I T, Nicholls S C, Holder A A. 1991. Proteolytic processing of the Plasmodium falciparum merozoite surface protein-1 produces a membrane-bound fragment containing two epidermal growth factor-like domains. Mol Biochem Parasitol 49(1):29-33.
Boes A, Spiegel H, Delbruck H, Fischer R, Schillberg S, Sack M. 2011. Affinity purification of a framework 1 engineered mouse/human chimeric IgA2 antibody from tobacco. Biotechnol Bioeng 108(12):2804-14.
Brochet, X., Lefranc, M.-P. & Giudicelli, V. IMGT/V-QUEST: the highly customized and integrated system for IG and TR standardized V-J and V-D-J sequence analysis. Nucleic acids research 36, W503-8 (2008).
Chen L, Lopaticki S, Riglar D T, Dekiwadia C, Uboldi A D, Tham W H, O'Neill M T, Richard D, Baum J, Ralph S A and others. 2011. An EGF-like protein forms a complex with PfRh5 and is required for invasion of human erythrocytes by Plasmodium falciparum. PLoS Pathog 7(9):e1002199.
Dayhoff, M. O. Atlas of Protein Sequence and Structure (Vol 5, Supplement 3). 353-358 (Natl Biomedical Research: 1979).
Epping R J, Goldstone S D, Ingram L T et al. An epitope recognized by inhibitory monoclonal antibodies that react with a 51 kilodalton merozoite surface antigen in Plasmodium falciparum. Exp Parasitol 1988; 81:90-6.
García-Basteiro A L, Bassat Q and Alonso P L. 2012. Approaching the Target: the Path Towards an Effective Malaria Vaccine. Mediterr J Hematol Infect Dis. 4(1):e2012015
Geysen, H. M., Meloen, R. H. & Barteling, S. J. Use of peptide synthesis to probe viral antigens for epitopes to a resolution of a single amino acid. Proceedings of the National Academy of Sciences of the United States of America 81, 3998-4002 (1984).
Gosselin, E. J., K. Wardwell, D. R. Gosselin, N. Alter, J. L. Fisher, and P. M. Guyre. 1992. Enhanced antigen presentation using human Fc gamma receptor (monocyte/macrophage)-specific immunogens. J. Immunol. 149:3477-3481.
Hügel F U, Pradel G and Frevert U. 1996. Release of malaria circumsporozoite protein into the host cell cytoplasm and interaction with ribosomes. Mol Biochem Parasitol. 81(2): 151-170
Ifediba T, Vanderberg J P. 1981. Complete in vitro maturation of Plasmodium falciparum gametocytes. Nature. 294(5839): 364-366
Kabat et al. 1991. Sequences of Proteins of Immunological Interest, National Institutes of Health, Bethesda, Md., ed, 5
Kariuki M M, Kiaira J K, Mulaa F K, Mwangi J K, Wasunna M K and Martin S K. 1998. Plasmodium falciparum: Purification of the various gametocyte developmental stages from in vitro-cultivated parasites. Am. J. Trop. Med. Hyg. 59(4): 505-508
Kaslow D C, Quakyi I A, Syin C, Raum M G, Keister D B, Coligan J E, McCutchan T F, Miller L H. 1988. A vaccine candidate from the sexual stage of human malaria that contains EGF-like domains. Nature 333 (6168):74-6.
Kusi, K. A. et al. Immunization with different PfAMA1 alleles in sequence induces clonal imprint humoral responses that are similar to responses induced by the same alleles as a vaccine cocktail in rabbits. Malaria journal 10, 40 (2011).
Livingstone, C. D. & Barton, G. J. Protein sequence alignments: a strategy for the hierarchical analysis of residue conservation. Computer applications in the biosciences: CABIOS 9, 745-56 (1993).
Mahajan, B., J. A. Berzofsky, et al. (2010). “Multiple antigen peptide vaccines against Plasmodium falciparum malaria.” Infect Immun 78(11): 4613-4624.
Makler, M. T. et al. Parasite lactate dehydrogenase as an assay for Plasmodium falciparum drug sensitivity. The American journal of tropical medicine and hygiene 48, 739-41 (1993).
Marshall V M, Silva A, Foley M, Cranmer S, Wang L, McColl D J, Kemp D J, Coppel R L. 1997. A second merozoite surface protein (MSP-4) of Plasmodium falciparum that contains an epidermal growth factor-like domain. Infect Immun 65(11):4460-7.
Marshall V M, Tieqiao W, Coppel R L. 1998. Close linkage of three merozoite surface protein genes on chromosome 2 of Plasmodium falciparum. Mol Biochem Parasitol 94(1):13-25.
Pelham H R. 1990. The retention signal for soluble proteins of the endoplasmic reticulum. Trends Biochem Sci 15(12):483-6.
Marshall V M, Silva A, Foley M et al. A second merozoite surface protein (MSP-4) of Plasmodium falciparum that contains an epidermal growth factor-like domain. Infect Immun 1997; 65:4460-7.
McCormick C J, Hollingdale M R and Taylor R. 2008. Sporozoite invasion assay. In: Methods in Malaria Research 5th Edition. K. Moll, I. Ljungström, H. Perlmann, A. Scherf and M. Wahlgren (Eds.). MR4/ATCC Manassas, Va. BioMalPar Paris, France. pp 138-140
Pachebat J A, Ling I T, Grainger M et al. The 22 kDa component of the protein complex on the surface of Plasmodium falciparum merozoites is derived from a larger precursor, merozoite surface protein 7. Mol Biochem Parasitol 2001; 117: 83-9.
Patarroyo M E, Amador R, Clavijo P, Moreno A, Guzman F, Romero P, et al. A synthetic vaccine protects humans against challenge with asexual blood stages of Plasmodium falciparum malaria. Nature. 1988; 332(6160):158-61
Pradel G, Hayton K, Aravind L, Iyer L M, Abrahamsen M S, Bonawitz A, Mejia C, Templeton T J. 2004. A multidomain adhesion protein family expressed in Plasmodium falciparum is essential for transmission to the mosquito. J. Exp. Med. 199(11): 1533-1544
Pradel G and Frevert U. 2001. Malaria sporozoites actively enter and pass through rat Kupffer cells prior to hepatocyte invasion. Hepatology. 33(5): 1154-11654. Chothia, C. et al. Conformations of immunoglobulin hypervariable regions. Nature 342, 877-83
Plassmeyer M L, Reiter K, Shimp R L, Jr., Kotova S, Smith P D, Hurt D E, House B, Zou X, Zhang Y, Hickman M and others. 2009. Structure of the Plasmodium falciparum circumsporozoite protein, a leading malaria vaccine candidate. J Biol Chem 284(39):26951-63.
Rathore D, Hrstka S C, Sacci J B Jr, De la Vega P, Linhardt R J, Kumar S and McCutchan T F. 2003. Molecular mechanism of host specificity in Plasmodium falciparum infection: role of circumsporozoite protein. J Biol Chem. 278(42): 40905-40910
Richards, J. S. and J. G. Beeson (2009). “The future for blood-stage vaccines against malaria.” Immunol Cell Biol 87(5): 377-390.
Roestenberg, M. et al. Safety and immunogenicity of a recombinant Plasmodium falciparum AMA1 malaria vaccine adjuvanted with Alhydrogel, Montanide ISA 720 or AS02. PloS one 3, e3960 (2008).
Sack M, Paetz A, Kunert R, Bomble M, Hesse F, Stiegler G, Fischer R, Katinger H, Stoeger E, Rademacher T. 2007. Functional analysis of the broadly neutralizing human anti-HIV-1 antibody 2F5 produced in transgenic BY-2 suspension cultures. FASEB J 21(8):1655-64.
Sambrook, J., Fritsch, E. F. & Maniatis, T. Molecular Cloning: A Laboratory Manual, Volume 1 to 3, 2nd edition. Sambrook J E F Fritsch and T Maniatis Molecular Cloning A Laboratory Manual Second Edition Vols 1 2 and 3 Cold Spring Harbor Laboratory Press Cold Spring Harbor New York N.Y. USA Illus Paper (1989).
Schwartz, L., G. V. Brown, et al. (2012). “A review of malaria vaccine clinical projects based on the WHO rainbow table.” Malar J 11: 11.
Smith, T. F. & Waterman, M. S. Comparison of biosequences. Advances in Applied Mathematics 2, 482-489 (1981).
Srinivasan P, Beatty W L, Diouf A, Herrera R, Ambroggio X, Moch J K, Tyler J S, Narum D L, Pierce S K, Boothroyd J C and others. 2011. Binding of Plasmodium merozoite proteins RON2 and AMA1 triggers commitment to invasion. Proc Natl Acad Sci USA 108(32):13275-80.
Tachibana M, Wu Y, Iriko H, Muratova O, MacDonald N J, Sattabongkot J, Takeo S, Otsuki H, Torii M, Tsuboi T. 2011. N-terminal prodomain of Pfs230 synthesized using a cell-free system is sufficient to induce complement-dependent malaria transmission-blocking activity. Clin Vacc
Tan K., Duquette M., Liu J., Dong Y., Zhang R., Joachimiak A., Lawler J., Wang J. 2002. Crystal structure of the TSP-1 type 1 repeats: A novel layered fold and its biological implication. J. Cell Biol. 159: 373-382.
Taylor, W. R. The classification of amino acid conservation. Journal of theoretical biology 119, 205-18 (1986).
Tossavainen H, Pihlajamaa T, Huttunen T K, Raulo E, Rauvala H, Permi P, Kilpelainen I. 2006. The layered fold of the TSR domain of P. falciparum TRAP contains a heparin binding site. Protein Sci 15(7):1760-8.
Trucco C, Fernadez-Reyes D, Howell S et al. The merozoite surface protein 6 gene codes for a 36 kDa protein associated with the Plasmodium falciparum merozoite surface protein-1 complex. Mol Biochem Parasitol 2001; 112:91-101.
Tucker R. P. 2004. The thrombospondin type 1 repeat family. Int. J. Biochem. Cell Biol. 36: 969-974.
Uchime O, Herrera R, Reiter K, Kotova S, Shimp R L, Jr., Miura K, Jones D, Lebowitz J, Ambroggio X, Hurt D E and others. 2012. Analysis of the Conformation and Function of the Plasmodium falciparum Merozoite Proteins MTRAP and PTRAMP. Eukaryot Cell 11(5):615-25.
Vaquero C, Sack M, Chandler J, Drossard J, Schuster F, Monecke M, Schillberg S, Fischer R. 1999. Transient expression of a tumor-specific single-chain fragment and a chimeric antibody in tobacco leaves. Proc Natl Acad Sci USA 96(20):11128-33.
van Schaijk, B. C. et al., (2008). “Gene disruption of Plasmodium falciparum p52 results in attenuation of malaria liver stage development in cultured primary human hepatocytes.” PLoS One 3(10): e3549.
Wasmuth, J., Daub, J., Peregrin-Alvarez, J. M., Finney, C. A., Parkinson, J. (2009). “The origins of apicomplexan sequence innovation.” Genome Res 19(7): 1202-1213.
Wong C, Sridhara S, Bardwell J C, Jakob U. 2000. Heating greatly speeds Coomassie blue staining and destaining. Biotechniques 28(3):426-8, 430, 432.
Beck, A. and J. M. Reichert (2011). “Therapeutic Fc-fusion proteins and peptides as successful alternatives to antibodies.” MAbs 3(5): 415-416.
Boes, A. et al., (2011). “Affinity purification of a framework 1 engineered mouse/human chimeric IgA2 antibody from tobacco.” Biotechnol Bioeng 108(12): 2804-2814.
Capon, D. J. et al., (1989). “Designing CD4 immunoadhesins for AIDS therapy.” Nature 337(6207): 525-531.
Mekhaiel, D. N. et al., (2011). “Polymeric human Fc-fusion proteins with modified effector functions.” Sci Rep 1: 124.
Pelham, H. R. (1990). “The retention signal for soluble proteins of the endoplasmic reticulum.” Trends Biochem Sci 15(12): 483-486.
Perez de la Lastra, J. M. et al., (2009). “Recent advances in the development of immunoadhesins for immune therapy and as anti-infective agents.” Recent Pat Antiinfect Drug Discov 4(3): 183-189.
Richards, J. S. and J. G. Beeson (2009). “The future for blood-stage vaccines against malaria.” Immunol Cell Biol 87(5): 377-390.
Sack, M. et al., (2007). “Functional analysis of the broadly neutralizing human anti-HIV-1 antibody 2F5 produced in transgenic BY-2 suspension cultures.” FASEB J 21(8): 1655-1664.
Vaquero, C. et al., (1999). “Transient expression of a tumor-specific single-chain fragment and a chimeric antibody in tobacco leaves.” Proc Natl Acad Sci USA 96(20): 11128-11133.

1. An isolated immunoassemblin (IA) protein complex suitable as a vaccine comprising at least three recombinant fusion protein units, wherein:
a) the first fusion protein unit comprises the immunoglobulin heavy chain constant domains C_{H}1 and C_{H}3 and a first antigen, wherein said first antigen is linked N-terminal or C-terminal to at least one of the immunoglobulin heavy chain constant domains (HC fusion polypeptide unit 1, HC unit 1);
b) the second fusion protein unit comprises an immunoglobulin light chain constant domain C_{L}, and a second antigen, wherein said second antigen is linked N-terminal or C-terminal to the C_{L}-domain (LC fusion polypeptide unit 1, LC unit 1);
c) the third fusion protein unit comprises:
i) the immunoglobulin heavy chain constant domains C_{H}1 and C_{H}3 and a third antigen, wherein said third antigen is fused N-terminal or C-terminal to the immunoglobulin heavy chain constant domains of said third fusion protein, or
ii) the third fusion protein unit comprises an immunoglobulin light chain constant domain C_{L}, and a third antigen, wherein said third antigen is fused N- or C-terminal to the C_{L}-domain; and
wherein said antigens of said three recombinant fusion protein units differ in their amino acid sequence.

2-3. (canceled)

4. The isolated IA protein complex according to claim 1, wherein the third fusion protein unit comprises the immunoglobulin heavy chain constant domains C_{H}1 and C_{H}3 and a third antigen (second HC fusion polypeptide unit 2, HC unit 2), wherein said third antigen is fused N-terminal or C-terminal to the immunoglobulin heavy chain constant domains of said third fusion protein.

5-6. (canceled)

7. The isolated IA protein complex according to claim 4, wherein said IA protein complex comprises a fourth recombinant fusion protein unit comprising an immunoglobulin light chain constant domain C_{L}, and a fourth antigen, wherein said fourth antigen is fused N- or C-terminal to the C_{L}-domain (second LC fusion polypeptide unit 2, LC unit 2).

8-17. (canceled)

18. The isolated IA protein complex according to claim 7, wherein
a) said first fusion protein unit comprises the immunoglobulin heavy chain constant domains C_{H}1 and C_{H}3 and a first antigen, wherein said first antigen is fused N-terminal to the C_{H}1-domain (HC unit 1);
b) said second fusion protein unit comprises an immunoglobulin light chain constant domain C_{L}, and a second antigen, wherein said second antigen is fused N-terminal to the C_{L}-domain (LC unit 1), and wherein said first and said second fusion protein unit are covalently linked to each other, in particular by at least one disulfide bond;
c) said third fusion protein unit comprises the immunoglobulin heavy chain constant domains C_{H}1 and C_{H}3 and a third antigen, wherein said third antigen is fused N-terminal to the C_{H}1-domain of said third fusion protein unit (HC unit 2), wherein said HC unit 1 and HC unit 2 are covalently linked to each other, in particular by at least one disulphide bond; and
d) said fourth fusion protein unit comprises an immunoglobulin light chain constant domain C_{L}, and a fourth antigen, wherein said fourth antigen is fused N-terminal to the C_{L}-domain of said fourth fusion protein (LC unit 2), and wherein said third and the fourth fusion protein unit are covalently linked to each other, in particular by at least one disulfide bond.

19. The isolated IA protein complex according to claim 7, wherein at least one of the fusion protein units comprise an additional antigen, wherein said additional antigens are linked N-terminal or C-terminal to the fusion protein unit.

20-26. (canceled)

27. The isolated IA protein complex according to claim 1, wherein the C_{L}-domain comprises the amino acid sequence of SEQ ID NO. 50.

28-29. (canceled)

30. The isolated IA protein complex according to claim 1, wherein said protein complex comprising antigens derived from two different pathogens.

31-33. (canceled)

34. The IA protein complex according to claim 1, wherein the antigens comprising an amino acid sequence selected from the group consisting of SEQ ID NO. 1 to SEQ ID NO. 31.

35. The isolated IA protein complex according to claim 1, wherein the antigens are derived from at least two different proteins presented on the surface of said parasite.

36-42. (canceled)

43. The isolated IA protein complex according to claim 1, comprising:
a) a first fusion protein unit (HC unit 1) having an amino acid sequence selected from the group of SEQ ID NO. 33 to SEQ ID NO. 52, SEQ ID NO. 67 to SEQ ID NO. 75 and SEQ ID NO. 78 to SEQ ID NO. 104; and
b) a second fusion protein unit (LC unit 1) having an amino acid sequence selected from the group of SEQ ID NO. 51 to SEQ ID NO. 66.

44. The isolated IA protein complex according to claim 1, wherein the two recombinant fusion proteins having the amino acid sequences of:
a) first fusion protein unit (HC unit 1), SEQ ID NO. 97; and
b) second fusion protein unit (LC unit 1), SEQ ID NO. 53.

45-46. (canceled)

47. The isolated IA protein complex according to claim 4, wherein
a) said first fusion protein unit (HC unit 1) comprises the antigens AMA1 GKO and CSP_TSR GKO, wherein the AMA1 GKO is N-terminal fused to the C_{H}1-domain and CSP_TSR GKO is C-terminal fused to the C_{H}3-domain, and wherein the Fc receptor binding portion comprised in the first fusion protein having an amino acid sequence that varies from the amino acid sequence of SEQ ID NO. 32 with at least two substitutions as compared with SEQ ID NO. 32, and wherein the substitutions occurs at position Glu356 and Asp399 of SEQ ID NO. 32, wherein the amino acids at position Glu356 and Asp399 are substituted with positive charged amino acids;
b) said second fusion protein unit (LC unit 1) comprises the antigen Pfs25 FKO, further wherein Pfs25 FKO is N-terminal fused to the C_{L}-domain; and
c) said third fusion protein unit (HC unit 2) comprises the antigen Rh2 GKO, that is N-terminally fused to the C_{H}1-domain, and wherein the Fc receptor binding portion comprised in the first fusion protein having an amino acid sequence that varies from the amino acid sequence of SEQ ID NO. 32 with at least two substitutions as compared with SEQ ID NO. 32, and wherein the substitutions occurs at position Lys392 and Lys 409 of SEQ ID NO. 32.

48. The isolated IA protein complex according to claim 4, comprising:
a) a first fusion protein unit (HC unit 1) having an amino acid sequence selected from the group consisting of SEQ ID NO. 33 to SEQ ID NO. 52, SEQ ID NO. 67 to SEQ ID NO. 75 and SEQ ID NO. 78 to SEQ ID NO. 104;
b) a second fusion protein unit (LC unit 1) having an amino acid sequence selected from the group consisting of SEQ ID NO. 51 to SEQ ID NO. 66; and
c) a third fusion protein unit (HC unit 2) having an amino acid sequence selected from the group consisting of SEQ ID NO. 33 to SEQ ID NO. 52, SEQ ID NO. 67 to SEQ ID NO. 75 and SEQ ID NO. 78 to SEQ ID NO. 104.

49-50. (canceled)

51. The isolated IA protein complex according to claim 7, comprising:
a) a first fusion protein unit (HC unit 1) having an amino acid sequence selected from the group consisting of SEQ ID NO. 33 to SEQ ID NO. 52, SEQ ID NO. 67 to SEQ ID NO. 75 and SEQ ID NO. 78 to SEQ ID NO. 104;
b) a second fusion protein unit (LC unit 1) having an amino acid sequence selected from the group consisting of SEQ ID NO. 51 to SEQ ID NO. 66;
c) a third fusion protein unit (HC unit 2) having an amino acid sequence selected from the group consisting of SEQ ID NO. 33 to SEQ ID NO. 52, SEQ ID NO. 67 to SEQ ID NO. 75 and SEQ ID NO. 78 to SEQ ID NO. 104; and
d) a fourth fusion protein unit (LC unit 2) having an amino acid sequence selected from the group consisting of SEQ ID NO. 51 to SEQ ID NO. 66.

52. The isolated IA protein complex according to claim 7, wherein
a) the first fusion protein unit (HC unit 1) comprises SEQ ID NO. 97;
b) the second fusion protein unit (LC unit 1) comprises SEQ ID NO. 53;
c) the third fusion protein unit (HC unit 2) comprises SEQ ID NO. 88, SEQ ID NO. 99 or SEQ ID NO. 100; and
d) the fourth fusion protein unit (LC unit 2) comprises SEQ ID NO. 53.

53. A nucleic acid molecule comprising a coding portion encoding one or more of the recombinant fusion proteins as defined in claim 1.

54. An expression vector comprising a nucleotide molecule according to claim 53.

55. A host cell comprising a vector according to claim 54, wherein the host cell comprises at least one or all nucleic acid molecules encoding one or more of the fusion proteins of the isolated protein complex according to claim 1.

56-59. (canceled)

60. A vaccine composition comprising an immunoassemblin protein complex according to claim 1, and a pharmaceutically acceptable carrier or an adjuvant.

61. A method of producing an isolated immunoassemblin (IA) protein suitable for a vaccine complex comprising the steps of:
a) culturing a host cell according to claim 55 in a suitable culture medium under suitable conditions to produce said recombinant fusion proteins, wherein the host cell comprises all nucleic acid molecules encoding the fusion proteins units of said isolated IA protein complex, and wherein said IA protein complex is formed in the host cell;
b) isolating said IA protein complex; and optionally
c) processing said IA protein complex.

62. A method of producing an isolated immunoassemblin (IA) protein suitable for a vaccine complex comprising the steps of:
(a) culturing of a plurality of host cells according to claim 55, in a suitable culture medium under suitable conditions to produce said recombinant fusion proteins;
(b) isolating said produced fusion proteins;
(c) mixing said fusion proteins to produce said IA protein complex;
(d) isolating said produced IA protein complex; and optionally
(e) processing the IA protein complex.

63-64. (canceled)

65. An isolated IA protein complex suitable as a vaccine comprising (i) at least two recombinant fusion proteins, (ii) at least two different antigens, (iii) at least one different homo- or hetero-oligomerization domain, wherein said first recombinant fusion protein comprise at least one homo- or hetero-oligomerization domain that is absent from said second recombinant fusion protein.

66-67. (canceled)
